(19) United States
(12) Patent Application Publication
(10) Pub. No.: US 2008/0242683 A1
(43) Pub. Date: Oct. 2, 2008

(54) Organic Compounds

(76) Inventors: Robin Alec Fairhurst, West Sussex (GB); Roger John Taylor, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/908,620

(22) PCT Filed: Mar. 13, 2006
(86) PCT No.: PCT/EP2006/002281

§ 371 Date: Sep. 14, 2007

(30) Foreign Application Priority Data
Mar. 14, 2005 (GB) 0505219.6

(51) Int. Cl. A61K 31/52; C07D 473/16

(52) U.S. Cl. 514/263.22; 544/277; 514/263.23

(57) ABSTRACT

Compounds of (I)

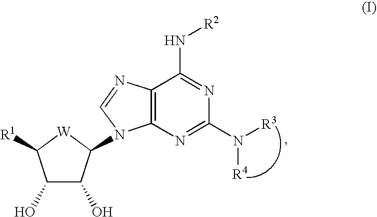

or stereoisomers or pharmaceutically acceptable salts thereof, where W, R¹, R², R³ and R⁴ have the meanings as indicated in the specification, are useful for treating conditions mediated by activation of the adenosine A_{2A} receptor, especially inflammatory or obstructive airways diseases. Pharmaceutical compositions that contain the compounds and a process for preparing the compounds are also described.

This application is the National Stage of Application No. PCT/EP2006/002281, filed on Mar. 13, 2006. The contents are incorporated herein by reference in their entirety.

This invention relates to organic compounds, their preparation and use as pharmaceuticals.

In one aspect, the present invention provides compounds of formula (I)

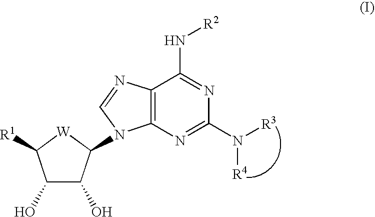

or stereoisomers or pharmaceutically acceptable salts thereof,
wherein
W is selected from CH₂ and O;
R¹ is selected from CH₂OH, CH₂—O—C₁-C₈-alkyl, C(O)—O—C₁-C₈-alkyl, C(O)NH₂, C(O)—NH—C₁-C₈-alkyl and a 3- to 10-membered heterocyclic ring containing at least one ring heteroatom selected from the group consisting of nitrogen, oxygen and sulphur, optionally substituted by C₁-C₈-alkyl;
R² is hydrogen or C₁-C₈-alkyl optionally substituted by hydroxy or C₆-C₁₀-aryl;
R³ and R⁴, together with the nitrogen atom to which they are attached, form a 3- to 10-membered heterocyclic group containing the indicated nitrogen atom as a ring heteroatom and optionally at least one ring heteroatom selected from the group consisting of nitrogen, oxygen and sulphur, optionally substituted by 0-3R⁵;
   R⁵ is selected from OH, C₁-C₈-alkyl optionally substituted by OH, C₁-C₈-alkoxy, C₇-C₁₄-aralkyl optionally substituted with OH, O—C₁-C₈-alkyl, halogen C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, O—C₁-C₈-alkyl or -halogen, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₈-alkylcarbonyl, C₁-C₈-alkoxycarbonyl, di(C₁-C₈-alkyl)aminocarbonyl, COOR^{5k}, C(O)R^{5l} and a 3- to 10-membered heterocyclic group containing at least one ring heteroatom selected from the group consisting of nitrogen, oxygen and sulphur optionally substituted by COOR^{5m};
   R^{5a}, R^{5b}, R^{5c}, R^{5f}, R^{5h} and R⁵ⁱ are, independently, H, C₁-C₈-alkyl or C₆-C₁₀-aryl;
   R^{5d}, R^{5e}, R^{5g} and R^{5j} are, independently, C₁-C₈-alkyl or a 3- to 10-membered heterocyclic group containing at least one ring heteroatom selected from the group consisting of nitrogen, oxygen and sulphur, optionally substituted by 0-3R⁶;
   R^{5k} is H, C₁-C₈-alkyl, C₆-C₁₀-aryl or a 3- to 10-membered heterocyclic group containing at least one ring heteroatom selected from the group consisting of nitrogen, oxygen and sulphur;
   R⁵ⁱ is C₁-C₈-alkyl, C₆-C₁₀-aryl, NHR⁷ or a 3- to 10-membered heterocyclic group containing at least one ring heteroatom selected from the group consisting of nitrogen, oxygen and sulphur;
   R^{5m} is H, C₁-C₈-alkyl or C₇-C₁₄-aralkyl;
   R⁶ is selected from OH, C₁-C₈-alkyl optionally substituted by OH, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, O—C₁-C₈-alkyl or -halogen, NR^{6a}R^{6b}, NHC(O)R^{6c}, NHS(O)₂R^{6d}, NHS(O)₂R^{6e}, NR^{6f}C(O)NR^{6g}R^{6h}, NR⁶ⁱC(O)OR^{6j}, C₁-C₈-alkylcarbonyl, C₁-C₈-alkoxycarbonyl, di(C₁-C₈-alkyl)aminocarbonyl, COOR^{6k}, C(O)R^{6l}, C(O)NHR^{6m} and a 3-10-membered heterocyclic group containing at least one ring heteroatom selected from the group consisting of nitrogen, oxygen and sulphur, optionally substituted by 0-3R⁸;
   R^{6a}, R^{6b}, R^{6c}, R^{6f}, R^{6h} and R⁶ⁱ are, independently, H, C₁-C₈-alkyl or C₆-C₁₀-aryl;
   R^{6d}, R^{6e}, R^{6g}, R^{6j} and R^{6m} are, independently, C₁-C₈-alkyl or a 3- to 10-membered heterocyclic group containing at least one ring heteroatom selected from the group consisting of nitrogen, oxygen and sulphur, optionally substituted by COOR⁹;
   R^{6k} is H, C₁-C₈-alkyl, C₆-C₁₀-aryl or a 3- to 10-membered heterocyclic group containing at least one ring heteroatom selected from the group consisting of nitrogen, oxygen and sulphur;
   R^{6l} is C₁-C₈-alkyl, C₆-C₁₀-aryl or a 3- to 10-membered heterocyclic group containing at least one ring heteroatom selected from the group consisting of nitrogen, oxygen and sulphur, optionally substituted by COOR¹⁰;
   R⁷ is COO R⁷ or a 3- to 10-membered heterocyclic group containing at least one ring heteroatom selected from the group consisting of nitrogen, oxygen and sulphur, optionally substituted by COOR^{7b}; and
   R^{7a}, R^{7b}, R⁸, R⁹ and R¹⁰ are selected from H, C₁-C₈-alkyl and C₇-C₁₄-aralkyl.

Terms used in the specification have the following meanings:

“Optionally substituted” means the group referred to can be substituted at one or more positions by any one or any combination of the radicals listed thereafter.

“Halo” or “halogen”, as used herein, may be fluorine, chlorine, bromine or iodine. Preferably halo is chlorine.

“C₁-C₈-Alkyl”, as used herein, denotes straight chain or branched alkyl having 1-8 carbon atoms. Preferably C₁-C₈-alkyl is C₁-C₄-alkyl.

“C₁-C₈-Alkoxy”, as used herein, denotes straight chain or branched alkoxy having 1-8 carbon atoms. Preferably, C₁-C₈-alkoxy is C₁-C₄-alkoxy.

“C₃-C₈-Cycloalkyl”, as used herein, denotes cycloalkyl having 3-8 ring carbon atoms, e.g., a monocyclic group, such as a cyclopropyl, cyclobutyl, cyclopentyl, cyclohexyl, cycloheptyl or cyclooctyl, any of which can be substituted by one or more, usually one or two, C₁-C₄-alkyl groups; or a bicyclic group, such as bicycloheptyl or bicyclooctyl. Preferably, C₃-C₈-cycloalkyl is C₃-C₆-cycloalkyl.

“C₁-C₈-Alkylamino” and “di(C₁-C₈-alkyl)amino”, as used herein, denote amino substituted respectively by one or two C₁-C₈-alkyl groups as hereinbefore defined, which may be the same or different. Preferably, C₁-C₈-alkylamino and di(C₁-C₈-alkyl)amino are respectively C₁-C₄-alkylamino and di(C₁-C₄-alkyl)amino.

“C₁-C₈-Alkylcarbonyl” and “C₁-C₈-alkoxycarbonyl”, as used herein, denote C₁-C₈-alkyl or C₁-C₈-alkoxy, respectively, as hereinbefore defined attached by a carbon atom to a carbonyl group.

Preferably, C₁-C₈-alkylcarbonyl and C₁-C₈-alkoxycarbonyl are C₁-C₄-alkylcarbonyl and C₁-C₄-alkoxycarbonyl, respectively.

“C₃-C₈-Cycloalkylcarbonyl”, as used herein, denotes C₃-C₈-cycloalkyl, as hereinbefore defined, attached by a carbon atom to a carbonyl group. Preferably, C₃-C₈-cycloalkylcarbonyl is C₃-C₅-cycloalkylcarbonyl.

“C₃-C₈-Cycloalkylamino”, as used herein, denotes C₃-C₈-cycloalkyl, as hereinbefore defined, attached by a carbon atom to the nitrogen atom of an amino group. Preferably, C₃-C₈-cycloalkylamino is C₃-C₅-cycloalkylamino.

“C₆-C₁₀-Aryl”, as used herein, denotes a monovalent carbocyclic aromatic group that contains 6-10 carbon atoms and which may be, e.g., a monocyclic group, such as phenyl; or a bicyclic group, such as naphthyl. Preferably, C₆-C₁₀-aryl is C₆-C₈-aryl, especially phenyl.

“C₇-C₁₄-Aralkyl”, as used herein, denotes alkyl, e.g., C₁-C₄-alkyl, as hereinbefore defined, substituted by C₆-C₁₀-aryl as hereinbefore defined. Preferably, C₇-C₁₄-aralkyl is C₇-C₁₀-aralkyl, such as phenyl-C₁-C₄-alkyl.

“C₁-C₈-Alkylaminocarbonyl” and “C₃-C₈-cycloalkylaminocarbonyl”, as used herein, denote C₁-C₈-alkylamino and C₃-C₈-cycloalkylamino, respectively, as hereinbefore defined, attached by a carbon atom to a carbonyl group. Preferably, C₁-C₈-alkylaminocarbonyl and C₃-C₈-cycloalkyl-aminocarbonyl are C₁-C₄-alkylaminocarbonyl and C₃-C₈-cycloalkylaminocarbonyl, respectively.

“C₆-C₁₀-Arylcarbonyl” and “C₇-C₁₄-arylkylcarbonyl”, as used herein, denote C₆-C₁₀-aryl and C₇-C₁₄-arylkyl, respectively, as hereinbefore defined, attached by a carbon atom to a carbonyl group. Preferably, C₆-C₁₀-arylcarbonyl and C₇-C₁₄-arylkylcarbonyl are C₆-C₈-arylcarbonyl and C₇-C₁₀-arylkylcarbonyl, respectively.

“C₃-C₁₅-Carbocyclic group”, as used herein, denotes a carbocyclic group having 3-15 ring carbon atoms, e.g., a monocyclic group, either aromatic or non-aromatic, such as a cyclopentyl, cyclohexyl, cycloheptyl, cyclooctyl or phenyl; or a bicyclic group, such as bicyclooctyl, bicyclononyl, bicyclodecyl, indanyl or indenyl, again any of which can be substituted by one or more, usually one or two, C₁-C₄-alkyl groups. Preferably the C₃-C₁₅-carbocyclic group is a C₅-C₁₀-carbocyclic group, especially phenyl, cyclohexyl or indanyl. The C₅-C₁₅-carbocyclic group can unsubstituted or substituted. Preferred substituents on the heterocyclic ring include halo, cyano, hydroxy, carboxy, amino, aminocarbonyl, nitro, C₁-C₁₀-alkyl, C₁-C₁₀-alkoxy and C₃-C₁₀-cycloalkyl, especially amino.

“3- to 10-Membered heterocyclic ring containing at least one ring heteroatom selected from the group consisting of nitrogen, oxygen and sulphur”, as used herein, may be, e.g., furan, pyrrole, pyrrolidine, pyrazole, imidazole, triazole, isotriazole, tetrazole, thiadiazole, isothiazole, oxadiazole, pyridine, piperidine, pyrazine, oxazole, isoxazole, pyrazine, pyridazine, pyrimidine, piperazine, pyrrolidine, morpholino, triazine, oxazine or thiazole. Preferred heterocyclic rings include piperazine, pyrrolidine, morpholino, imidazole, isotriazole, pyrazole, tetrazole, thiazole, thiadiazole, pyridine, piperidine, pyrazine, furan, oxazole, isoxazole, oxadiazole and azetidine. The 3-to-10-membered heterocyclic ring can be unsubstituted or substituted. Preferred substituents include halo, cyano, oxo, hydroxy, carboxy, nitro, C₁-C₈-alkyl, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl. Especially preferred substituents include halo, oxo, C₁-C₄-alkyl, C₁-C₄-alkylcarbonyl, hydroxy-C₁-C₄-alkyl, C₁-C₄-haloalkyl, amino-C₁-C₄-alkyl and amino(hydroxy)C₁-C₄-alkyl.

Throughout this specification and in the claims that follow, unless the context requires otherwise, the word “comprise”, or variations, such as “comprises” or “comprising”, will be understood to imply the inclusion of a stated integer or step or group of integers or steps but not the exclusion of any other integer or step or group of integers or steps.

Preferred compounds of formula (I) or stereoisomers or pharmaceutically acceptable salts thereof,

wherein
W is selected from CH₂ and O;
R¹ is selected from CH₂OH, C(O)—NH—C₁-C₈-alkyl and a 3- or 10-membered heterocyclic ring containing at least one ring heteroatom selected from the group consisting of nitrogen, oxygen and sulphur optionally substituted by C₁-C₈-alkyl;
R² is hydrogen or C₁-C₈-alkyl optionally substituted by C₆-C₁₀-aryl;
R³ and R⁴, together with the nitrogen atom to which they are attached, form a 3- to 10-membered heterocyclic group containing the indicated nitrogen atom as a ring heteroatom and optionally at least one ring heteroatom selected from the group consisting of nitrogen, oxygen and sulphur, optionally substituted by 0-3R⁵;
R⁵ is selected from OH, C₁-C₈-alkyl optionally substituted by OH, or C₁-C₈-alkoxy, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or halogen, O—C₆-C₁₀-aryl optionally substituted by OH, C₁-C₈-alkyl, O—C₁-C₈-alkyl or halogen, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₈-alkylcarbonyl, C₁-C₈-alkoxycarbonyl, di(C₁-C₈-alkyl)aminocarbonyl, COOR^{5k}, C(O)R^{5l} and a 3- to 10-membered heterocyclic group containing at least one ring heteroatom selected from the group consisting of nitrogen, oxygen and sulphur optionally substituted by COOR^{5m};
   R^{5a}, R^{5b}, R^{5c}, R^{5f}, R^{5h} and R⁵ⁱ are, independently, H, C₁-C₈-alkyl or C₆-C₁₀-aryl;
   R^{5d}, R^{5e}, R^{5g} and R^{5j} are, independently, C₁-C₈-alkyl or a 3- to 10-membered heterocyclic group containing at least one ring heteroatom selected from the group consisting of nitrogen, oxygen and sulphur, optionally substituted by 0-3R⁶;
   R^{5k} is H, C₁-C₈-alkyl, C₆-C₁₀-aryl or a 3- to 10-membered heterocyclic group containing at least one ring heteroatom selected from the group consisting of nitrogen, oxygen and sulphur;
   R^{5l} is C₁-C₈-alkyl, C₆-C₁₀-aryl, NHR⁷ or a 3- to 10-membered heterocyclic group containing at least one ring heteroatom selected from the group consisting of nitrogen, oxygen and sulphur;
   R^{5m} is H, C₁-C₈-alkyl or C₇-C₁₄-aralkyl;
   R⁶ is selected from OH, C₁-C₈-alkyl optionally substituted by OH, C₇-C₁₄-aralkyl optionally substituted with OH, O—C₁-C₈-alkyl, C₆-C₁₀-aryl or O—C₆-C₁₀-aryl, C₁-C₈-alkoxy, C₆-C₁₀-aryl optionally substituted by OH, C₁-C₈-alkyl, O—C₁-C₈-alkyl or halogen, O—C₆-C₁₀-aryl optionally substituted by OH, C₁-C₈-alkyl, O—C₁-C₈-alkyl or halogen, NR^{6a}R^{6b}, NHC(O)R^{6c}, NHS(O)₂R^{6d}, NHS(O)₂R^{6e}, NR^{6f}C(O)NR^{6g}R^{6h}, NR⁶ⁱC(O)OR^{6j}, C₁-C₈-alkylcarbonyl, C₁-C₈-alkoxycarbonyl, di(C₁-C₈-alkyl)aminocarbonyl, COOR^{6k}, C(O)R^{6l}, C(O)NHR^{6m} and a 3- to 10-membered heterocyclic group containing at least one ring heteroatom selected from the group consisting of nitrogen, oxygen and sulphur, optionally substituted by 0-3R⁸;
   R^{6a}, R^{6b}, R^{6c}, R^{6f}, R^{6h} and R⁶ⁱ are, independently, H, C₁-C₈-alkyl or C₆-C₁₀-aryl;
   R^{6d}, R^{6e}, R^{6g}, R^{6j} and R^{6m} are, independently, C₁-C₈-alkyl or a 3- to 10-membered heterocyclic group containing at least one ring heteroatom selected from the group consisting of nitrogen, oxygen and sulphur, optionally substituted by 0-3R⁹;
   R^{6k} is H, C₁-C₈-alkyl, C₆-C₁₀-aryl or a 3- to 10-membered heterocyclic group containing at least one ring heteroatom selected from the group consisting of nitrogen, oxygen and sulphur;
   R^{6l} is C₁-C₈-alkyl, C₆-C₁₀-aryl or a 3- to 10-membered heterocyclic group containing at least one ring heteroatom selected from the group consisting of nitrogen, oxygen and sulphur, optionally substituted by COOR¹⁰;
   R⁷ is COOR^{7a} or a 3- to 10-membered heterocyclic group containing at least one ring heteroatom selected from the group consisting of nitrogen, oxygen and sulphur, optionally substituted by COOR^{7b}; and
   R^{7a}, R^{7b}, R⁸, R⁹ and R¹⁰ are selected from H, C₁-C₈-alkyl and C₇-C₁₄-aralkyl.
      Especially preferred compounds of the present invention include compounds of the formula (II) or stereoisomers or pharmaceutically acceptable salts thereof,

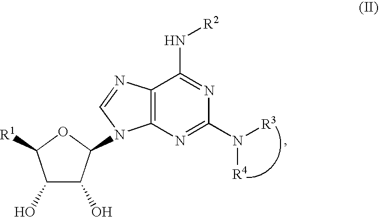

wherein
R¹ is selected from CH₂OH, C(O)—NH—C₁-C₄-alkyl and a 3- to 10-membered heterocyclic ring containing at least one ring heteroatom selected from the group consisting of nitrogen, oxygen and sulphur optionally substituted by C₁-C₈-alkyl;
R² is hydrogen or C₁-C₄-alkyl optionally substituted by C₆-C₈-aryl;
R³ and R⁴, together with the nitrogen atom to which they are attached, form a 3- to 10-membered heterocyclic group containing the indicated nitrogen atom as a ring heteroatom and optionally at least one other ring nitrogen atom, optionally substituted by at least one heteroatom selected from the group consisting of nitrogen, oxygen and sulphur, optionally substituted by 0-3R⁵, the heterocyclic group being saturated or comprising a saturated heterocyclic ring fused to a carbocyclic ring or being a 5-membered unsaturated group;
   R⁵ is selected from OH, C₁-C₄-alkyl optionally substituted by OH, C₁-C₄-alkoxy, C₆-C₁₀-aryl optionally substituted by halogen, O—C₆-C₁₀-aryl optionally substituted by halogen, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₄-alkylcarbonyl, C₁-C₄-alkoxycarbonyl, di(C₁-C₄-alkyl)aminocarbonyl, COOR^{5k}, C(O)R^{5l} and a 3- to 10-membered heterocyclic group containing at least one ring heteroatom selected from the group consisting of nitrogen, oxygen and sulphur optionally substituted by COOR^{5m};
   R^{5a}, R^{5b}, R^{5c}, R^{5f}, R^{5h} and R⁵ⁱ are, independently, H, C₁-C₄-alkyl or C₆-C₁₀-aryl;
   R^{5d}, R^{5e}, R^{5g} and R^{5j} are, independently, C₁-C₄-alkyl or a 3- to 10-membered heterocyclic group containing at least one ring heteroatom selected from the group consisting of nitrogen, oxygen and sulphur, optionally substituted by 0-3R⁶;
   R^{5k} is H, C₁-C₄-alkyl or C₆-C₁₀-aryl;
   R^{5l} is C₁-C₄-alkyl, C₆-C₁₀-aryl, NHR⁷ or a 3- to 10-membered heterocyclic group containing at least one ring heteroatom selected from the group consisting of nitrogen, oxygen and sulphur;
   R^{5m} is H, C₁-C₈-alkyl or C₇-C₁₄-aralkyl;
   R⁶ is selected from OH, C₁-C₄-alkyl optionally substituted by OH, C₆-C₁₀-aryl optionally substituted by OH, C₁-C₄-alkyl, O—C₁-C₄-alkyl or halogen, O—C₆-C₁₀-aryl optionally substituted by OH, C₁-C₄-alkyl, O—C₁-C₄-alkyl or halogen, NR^{6a}R^{6b}, NHC(O)R^{6c}, NHS(O)₂R^{6d}, NHS(O)₂R^{6e}, NR^{6f}C(O)NR^{6g}R^{6h}, NR⁶ⁱC(O)OR^{6j}, C₁-C₄-alkylcarbonyl, C₁-C₈-alkoxycarbonyl, di(C₁-C₄-alkyl)aminocarbonyl, COOR^{6k} and C(O)R^{6l}, C(O)NHR^{6m} and a 3- to 10-membered heterocyclic group containing at least one ring heteroatom selected from the group consisting of nitrogen, oxygen and sulphur, optionally substituted by 0-3R⁸;
   R^{6a}, R^{6b}, R^{6c}, R^{6f}, R^{6h} and R⁶ⁱ are, independently, H, C₁-C₄-alkyl or C₆-C₁₀-aryl;
   R^{6d}, R^{6e}, R^{6g}, R^{6j} and R^{6m} are, independently, C₁-C₄-alkyl or a 3- to 10-membered heterocyclic group containing at least one ring heteroatom selected from the group consisting of nitrogen, oxygen and sulphur, optionally substituted by 0-3R⁹;
   R^{6k} is H, C₁-C₄-alkyl, C₆-C₁₀-aryl or a 3- to 10-membered heterocyclic group containing at least one ring heteroatom selected from the group consisting of nitrogen, oxygen and sulphur;
   R^{6l} is C₁-C₄-alkyl, C₆-C₁₀-aryl or a 3- to 10-membered heterocyclic group containing at least one ring heteroatom selected from the group consisting of nitrogen, oxygen and sulphur, optionally substituted by COOR¹⁰;
   R⁷ is COOR^{7a} or a 3- to 10-membered heterocyclic group containing at least one ring heteroatom selected from the group consisting of nitrogen, oxygen and sulphur, optionally substituted by COOR^{7a}; and
   R^{7a}, R^{7b}, R⁸, R⁹ and R¹⁰ are selected from H, C₁-C₄-alkyl and C₇-C₁₄-aralkyl.

Especially preferred specific compounds of formula (I) are those described hereinafter in the Examples.

The compounds represented by formula (I) may be capable of forming acid addition salts, particularly pharmaceutically acceptable acid addition salts. Pharmaceutically acceptable acid addition salts of the compound of formula (I) include those of inorganic acids, e.g., hydrohalic acids, such as hydrofluoric acid, hydrochloric acid, hydrobromic acid or hydroiodic acid, nitric acid, sulfuric acid or phosphoric acid; and organic acids, e.g., aliphatic monocarboxylic acids, such as formic acid, acetic acid, trifluoroacetic acid, propionic acid and butyric acid; aliphatic hydroxy acids, such as lactic acid, citric acid, tartaric acid or malic acid; dicarboxylic acids, such as maleic acid or succinic acid; aromatic carboxylic acids, such as benzoic acid, p-chlorobenzoic acid, diphenylacetic acid, para-biphenyl benzoic acid or triphenylacetic acid; aromatic hydroxy acids, such as o-hydroxybenzoic acid, p-hydroxybenzoic acid, 1-hydroxynaphthalene-2-carboxylic acid, pamoic acid or 3-hydroxynaphthalene-2-carboxylic acid; cinnamic acids, such as 3-(2-naphthalenyl)propenoic acid, para-methoxy cinnamic acid or para-methyl cinnamic acid; and sulfonic acids, such as methanesulfonic acid or benzenesulfonic acid. These salts may be prepared from compounds of formula (I) by known salt-forming procedures.

Compounds of formula (I) which may contain acidic, e.g., carboxyl, groups, are also capable of forming salts with bases, in particular pharmaceutically acceptable bases, such as those well-known in the art; suitable such salts include metal salts, particularly alkali metal or alkaline earth metal salts, such as sodium, potassium, magnesium or calcium salts; or salts with ammonia or pharmaceutically acceptable organic amines or heterocyclic bases, such as ethanolamines, benzylamines or pyridine. These salts may be prepared from compounds of formula (Ia) by known salt-forming procedures.

Stereoisomers are those compounds where there is an asymmetric carbon atom. The compounds exist in individual optically active isomeric forms or as mixtures thereof, e.g., as diastereomeric mixtures. The present invention embraces both individual optically active R and S isomers, as well as mixtures thereof.

### Synthesis

Another embodiment of the present invention, provides a process for the preparation of compounds of formula (I), in free or pharmaceutically acceptable salt form, which comprises the steps of:

(i) reacting a compound of formula (III)

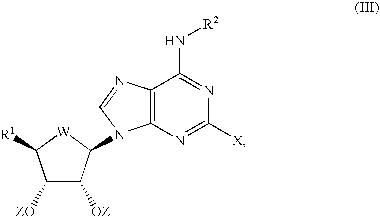

wherein
R¹, R² and W are as defined in claim 1;
Z is H or a protecting group; and
X is a leaving group,
   with a compound of formula (IV)

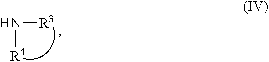

wherein
R³ and R⁴ are as defined in claim 1; and
removing any protecting groups and recovering the resultant compound of formula (I), in free or pharmaceutically acceptable salt form.

The compound of formula (III) may be prepared by reacting a compound of formula (V)

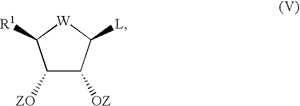

wherein
R¹, Z and W are as defined in claim 1; and
L represents a leaving group or a protected derivative thereof with a 2,6-dihalopurine, e.g., 2,6-dichloropurine,
   to provide a compound of formula (VI)

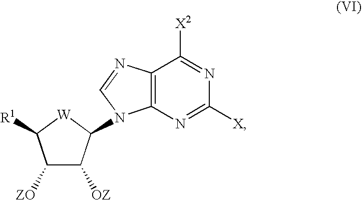

wherein
R¹, Z and W are defined in claim 1; and
X and X² are halogen.

Compound of formula (VI) can be reacted with R²NH₂ under conventional conditions to provide compound of formula (III).

The compounds of formula (I) can be prepared, e.g., using the reactions and techniques described below and in the Examples. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula (I) into another compound of formula (I). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such is alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991). It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practioner is Protective Groups In Organic Synthesis, Greene and Wuts, Eds., Wiley and Sons (1999).

Generally, compounds described in the scope of this patent application can be synthesized by the routes described in Schemes 1-5 and the Examples.

In Scheme 1, compounds of formula (I) can be prepared through two sequential nucleophilic aromatic substitution reactions to displace, e.g., chlorine atoms selectively and sequentially at the 6-position, to provide intermediate 2. Subsequent nucleophilic substitution at the 2-position with an appropriate amine provides compounds of formula (I). These reactions can be carried out either in the presence, or absence, of a base in addition to the reacting amine. A deprotection step may, or may not be necessary depending on the nature of the protecting group, if present.

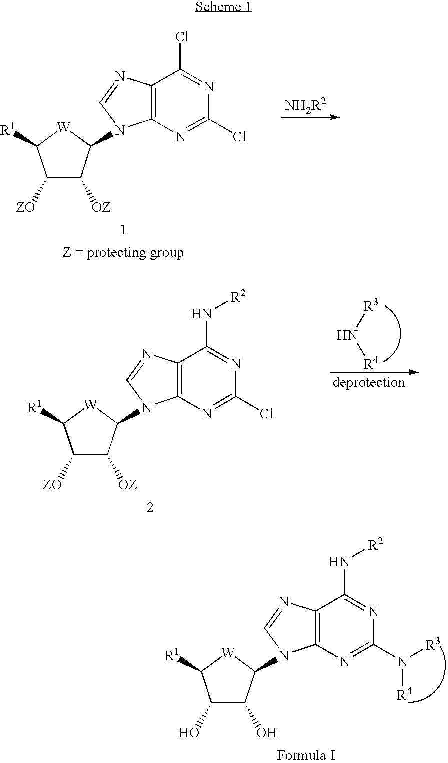

For instance, in Scheme 2, intermediate 3 or intermediate AD as referred to in the Examples, is synthesized in accordance with the procedures outlined in the Examples, can be reacted with an amine through microwave or conventional heating described in the Examples to generate compound 4.

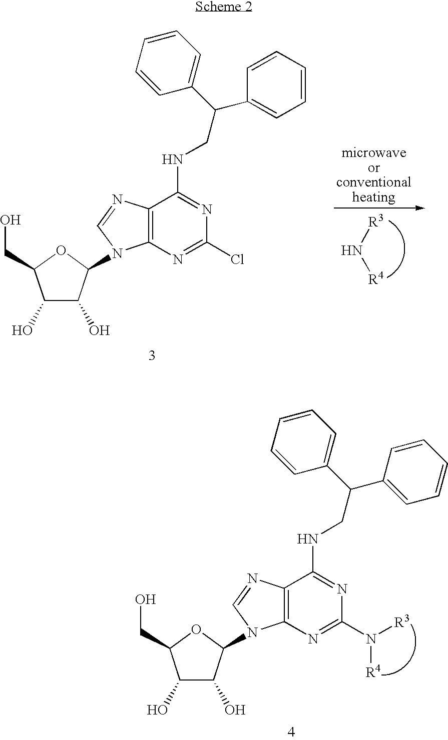

Also, in Scheme 3, compounds with nitro substituents, such as intermediate 5, or intermediate AC, as referred to in the Examples, is synthesized according to the procedures outlined in the Examples, can be reacted with amines similar to the procedure of Scheme 2 to provide compound 6.

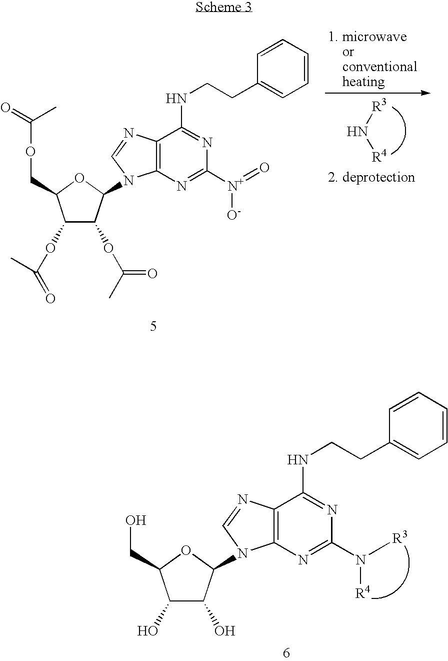

In Scheme 4 compounds with amide substituents are similarly generated as described in Schemes 2 and 3. For instance, intermediate 7, made according to the procedures outlined in WO 96/02553 and the J Med Chem, Vol. 33, No. 7, pp. 1919-1924 (1990), can be reacted with an amine under microwave heating conditions to provide compound 8.

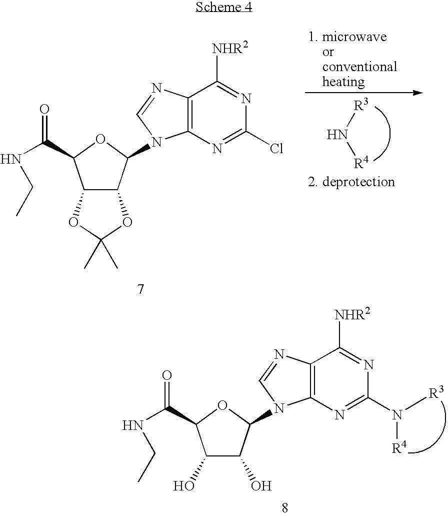

Also, purine derivative compounds with heterocyclic groups can be generated similar to the procedures outlined in Schemes 1-4 and the Examples. In Scheme 5, intermediate 9, where R¹ is a substituted tetrazole or a substituted isoxazole, such as ethyl substituted tetrazole or ethyl substituted isoxazole, can be generated according to the procedures outlined in WO 99/38877 and WO 98/28319. Intermediate 9 can then be reacted with an amine to provide compound 10.

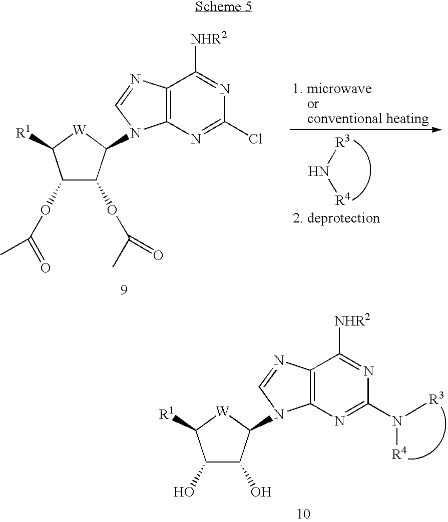

Compounds of formula (I), in free form, may be converted into salt form, and vice versa, in a conventional manner. The compounds in free or salt form can be obtained in the form of hydrates or solvates containing a solvent used for crystallisation. Compounds of formula (I) can be recovered from reaction mixtures and purified in a conventional manner. Isomers, such as stereoisomers, may be obtained in a conventional manner, e.g., by fractional crystallisation or asymmetric synthesis from correspondingly asymmetrically substituted, e.g., optically active, starting materials.

### Pharmacological Activity

Compounds of formula (I) and their pharmaceutically acceptable salts are useful as pharmaceuticals. In particular, they activate the adenosine A2A receptor, i.e., they act as A2A receptor agonists. Their properties as A2A agonists may be demonstrated using the method described by Murphree et al., Mol Pharmacol, Vol. 61, pp. 455-462 (2002).

Compounds of the Examples hereinbelow have Ki values below 5.0 μM in the above assay. For example, the compounds of Examples 2, 7, 9, 11, 13, 22, 24, 65, 77, 108, and 122 have Ki values of 0.61, 0.19, 0.16, 0.012, 0.054, 0.0005, 0.059, 0.002, 0.006, 0.005, and 0.004 μM respectively.

Having regard to their activation of the adenosine A2A receptor, compounds of formula (I), in free or pharmaceutically acceptable salt form, hereinafter alternately referred to as “agents of the invention”, are useful in the treatment of conditions which respond to the activation of the adenosine A2A receptor, particularly inflammatory or allergic conditions. Treatment in accordance with the invention may be symptomatic or prophylactic.

Accordingly, agents of the invention are useful in the treatment of inflammatory or obstructive airways diseases, resulting, e.g., in reduction of tissue damage, airways inflammation, bronchial hyperreactivity, remodeling or disease progression. Inflammatory or obstructive airways diseases and conditions to which the present invention is applicable include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular, other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis including, e.g., aluminosis, anthracosis, asbestosis, chalicosis, ptilosis, siderosis, silicosis, tabacosis and byssinosis.

Other inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma, asthma induced following bacterial infection and cystic fibrosis. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory, e.g., corticosteroid; or bronchodilatory. Prophylactic benefit in asthma may, in particular, be apparent in subjects prone to “morning dipping”. “Morning dipping” is a recognised asthmatic syndrome, common to a substantial percentage of asthmatics and characterised by asthma attack, e.g., between the hours of about 4-6 am, i.e., at a time normally substantially distant from any previously administered symptomatic asthma therapy.

Having regard to their anti-inflammatory activity, in particular in relation to inhibition of eosinophil activation, agents of the invention are also useful in the treatment of eosinophil related disorders, e.g., eosinophilia, in particular, eosinophil-related disorders of the airways, e.g., involving morbid eosinophilic infiltration of pulmonary tissues, including hyper-eosinophilia as it effects the airways and/or lungs, as well as, e.g., eosinophil-related disorders of the airways consequential or concomitant to Löffler's syndrome; eosinophilic pneumonia; parasitic, in particular, metazoan, infestation including tropical eosinophilia; bronchopulmonary aspergillosis; polyarteritis nodosa including Churg-Strauss syndrome; eosinophilic granuloma; and eosinophil-related disorders affecting the airways occasioned by drug-reaction.

Agents of the invention are also useful in the treatment of inflammatory or allergic conditions of the skin, e.g., psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita and other inflammatory or allergic conditions of the skin.

Agents of the invention may also be used for the treatment of other diseases or conditions, in particular, diseases or conditions having an inflammatory component, e.g., treatment of diseases and conditions of the eye, such as conjunctivitis, keratoconjunctivitis sicca and vernal conjunctivitis; diseases affecting the nose including allergic rhinitis; and inflammatory disease in which autoimmune reactions are implicated or having an autoimmune component or aetiology including autoimmune haematological disorders, e.g., haemolytic anaemia, aplastic anaemia, pure red cell anaemia and idiopathic thrombocytopenia; systemic lupus erythematosus; polychondritis; sclerodoma; Wegener granulamatosis; dermatomyositis; chronic active hepatitis; myasthenia gravis; Steven-Johnson syndrome; idiopathic sprue; autoimmune inflammatory bowel disease, e.g., ulcerative colitis and Crohn's disease; endocrine opthalmopathy; Grave's disease; sarcoidosis; alveolitis; chronic hypersensitivity pneumonitis; multiple sclerosis; primary billiary cirrhosis; uveitis (anterior and posterior); keratoconjunctivitis sicca and vernal keratoconjunctivitis; interstitial lung fibrosis; psoriatic arthritis; and glomerulonephritis (with and without nephrotic syndrome, e.g., including idiopathic nephrotic syndrome or minal change nephropathy).

Other diseases or conditions which may be treated with agents of the invention include diabetes, e.g., diabetes mellitus type I (juvenile diabetes) and diabetes mellitus type II; diarrheal diseases; ischemia/reperfusion injuries; retinopathy, such as diabetic retinopathy or hyperbaric oxygen-induced retinopathy; conditions characterised by elevated intraocular pressure or secretion of ocular aqueous humor, such as glaucoma; ischemic tissue/organ damage from reperfusion; and bedsores.

The effectiveness of an agent of the invention in inhibiting inflammatory conditions, e.g., an inflammatory airways diseases, may be demonstrated in an animal model, e.g., a mouse or at model, of airways inflammation or other inflammatory conditions, e.g., as described by Szarka et al., J Immunol Methods, Vol. 202, pp. 49-57 (1997); Renzi et al., Am Rev Respir Dis, Vol. 148, pp. 932-939 (1993); Tsuyuki et al., J Clin Invest, Vol. 96, pp. 2924-2931 (1995); Cernadas et al, Am J Respir Cell Mol Biol, Vol. 20, pp. 1-8 (1999); and Fozard et al., Er J Pharmacol, Vol. 438, pp. 183-188 (2002).

The agents of the invention are also useful as co-therapeutic agents for use in combination with other drug substances, such as anti-inflammatory, bronchodilatory, antihistamine or anti-tussive drug substances, particularly in the treatment of obstructive or inflammatory airways diseases, such as those mentioned hereinbefore, e.g., as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance.

Accordingly the invention includes a combination of an agent of the invention as hereinbefore described with an anti-inflammatory, bronchodilatory, antihistamine or anti-tussive drug substance, said agent of the invention and said drug substance being in the same or different pharmaceutical composition.

Suitable anti-inflammatory drugs include steroids, in particular, glucocorticosteroids, such as budesonide, beclamethasone dipropionate, fluticasone propionate, ciclesonide or mometasone furoate; or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTD4 antagonists, such as montelukast and zafirlukast; PDE4 inhibitors, such as cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™ CC-10004 (Celgene), VM554/UM565 (Vernalis), T440 (Tanabe), KW-4490 (Kyowa Hakko Kogyo) and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; adenosine A2B receptor antagonists, such is those described in WO 02/42298; and beta (β)-2 adrenoceptor agonists, such as albuterol salbutamol), metaproterenol, terbutaline, salmeterol fenoterol, procaterol, and especially, formoterol, carmoterol and pharmaceutically acceptable salts thereof, and compounds (in free or salt or solvate form) of formula (I) of WO 00/75114, which document is incorporated herein by reference, preferably compounds of the Examples thereof, especially a compound of formula

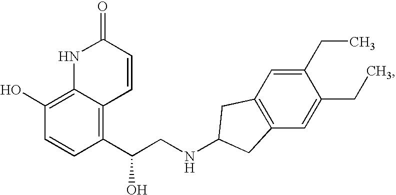

corresponding to indacaterol and pharmaceutically acceptable salts thereof, as well as compounds (in free or salt or solvate form) of formula (I) of WO 04/16601, and also compounds of EP 1440966, JP 05025045, WO 93/18007, WO 99/64035, US 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618, WO 04/46083, WO 04/80964, WO 04/108765 and WO 04/108676.

Suitable bronchodilatory drugs include anti-cholinergic or anti-muscarinic agents, in particular, ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422 and WO 04/05285.

Suitable dual anti-inflammatory and bronchodilatory drugs include dual β-2 adrenoceptor agonist/muscarinic antagonists, such as those disclosed in US 2004/0167167, WO 04/74246 and WO 04/74812.

Suitable anti-histamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine, as well as those disclosed in JP 2004107299, WO 03/099807 and WO 04/026841.

Other useful combinations of agents of the invention with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists, such as Schering-Plough antagonists SC-351125, SCH-55700 and SCH-D; Takeda antagonists, such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770); and CCR-5 antagonists described in U.S. Pat. No. 6,166,037 (particularly Claims 18 and 19), WO 00/66558 (particularly Claim 8), WO 00/66559 (particularly Claim 9), WO 04/018425 and WO 04/026873.

In accordance with the foregoing, the invention also provides a method for the treatment of a condition responsive to activation of the adenosine A2A receptor, e.g., an inflammatory or allergic condition, particularly an inflammatory or obstructive airways disease, which comprises administering to a subject, particularly a human subject, in need thereof a compound of formula (I), in free form or in the form of a pharmaceutically acceptable salt. In another aspect, the invention provides a compound of formula (I), in free form or in the form of a pharmaceutically acceptable salt, for use in the manufacture of a medicament for the treatment of a condition responsive to activation of the adenosine A2A receptor, particularly an inflammatory or obstructive airways disease.

The agents of the invention may be administered by any appropriate route, e.g., orally, e.g., in the form of a tablet or capsule; parenterally, e.g., intravenously; by inhalation, e.g., in the treatment of inflammatory or obstructive airways disease; intranasally, e.g., in the treatment of allergic rhinitis; topically to the skin, e.g., in the treatment of atopic dermatitis; or rectally, e.g., in the treatment of inflammatory bowel disease.

In a further aspect, the invention also provides a pharmaceutical composition comprising a compound of formula (I), in free form or in the form of a pharmaceutically acceptable salt, optionally together with a pharmaceutically acceptable diluent or carrier therefor. The composition may contain a co-therapeutic agent, such as an anti-inflammatory, bronchodilatory, anti-histamine or anti-tussive drug, as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations.

When the composition comprises an aerosol formulation, it preferably contains, e.g., a hydro-fluoro-alkane (HFA) propellant, such as HFA134a or HFA227 or a mixture of these, and may contain one or more co-solvents known in the art such as ethanol (up to 20% by weight); and/or one or more surfactants, such as oleic acid or sorbitan trioleate; and/or one or more bulking agents, such as lactose. When the composition comprises a dry powder formulation, it preferably contains, e.g., the compound of formula (I) having a particle diameter up to 10 microns, optionally together with a diluent or carrier, such as lactose, of the desired particle size distribution and a compound that helps to protect against product performance deterioration due to moisture, e.g., magnesium stearate. When the composition comprises a nebulised formulation, it preferably contains, e.g., the compound of formula (I) either dissolved, or suspended, in a vehicle containing water, a co-solvent, such as ethanol or propylene glycol and a stabiliser, which may be a surfactant.

The invention includes:
a) a compound of formula (I) in inhalable form, e.g., in an aerosol or other atomisable composition or in inhalable particulate, e.g., micronised, form;
b) an inhalable medicament comprising a compound of formula (I) in inhalable form;
c) a pharmaceutical product comprising a compound of formula (I) in inhalable form in association with an inhalation device; and
d) an inhalation device containing a compound of formula (I) in inhalable form.

Dosages of compounds of formula (I) employed in practising the present invention will of course vary depending, e.g., on the particular condition to be treated, the effect desired and the mode of administration. In general, suitable daily dosages for administration by inhalation are of the order of 0.005-10 mg, while for oral administration suitable daily doses are of the order of 0.05-100 mg.

The invention is illustrated by the following Examples.

### EXAMPLES 1-128

Compounds of formula (Ia)

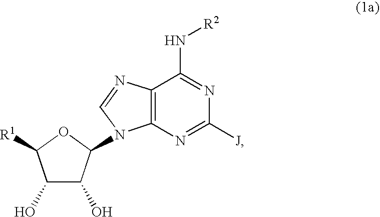

are shown in Table 1. Methods for preparing such compounds are described hereinafter. Table 1 also shows mass spectrometry, MH+ (ESI+), data.

TABLE 1

| Ex. | R¹ | R² | J | MH⁺ |
|---|---|---|---|---|
| 1 | 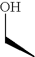 | 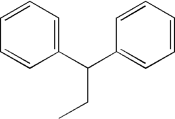 | 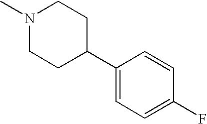 | 625 |
| 2 | 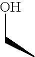 | 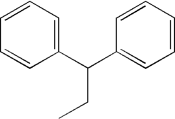 | 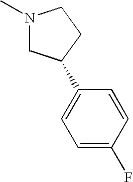 | 611 |
| 3 | 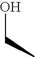 | 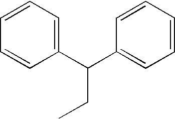 | 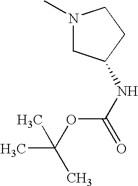 | 632 |
| 4 | 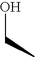 | 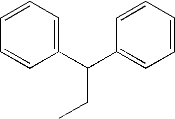 | 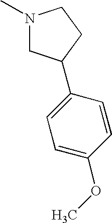 | 623 |
| 5 | 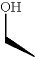 | 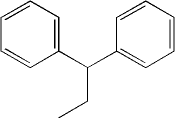 | 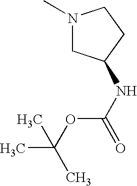 | 632 |
| 6 | 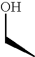 | 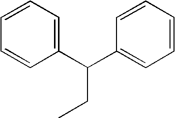 | 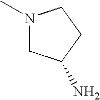 | 532 |
| 7 | 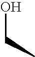 | 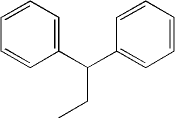 | 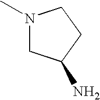 | 532 |
| 8 | 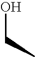 | 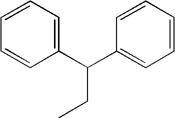 | 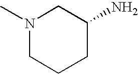 | 547 |
| 9 | 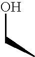 | 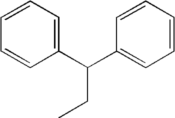 | 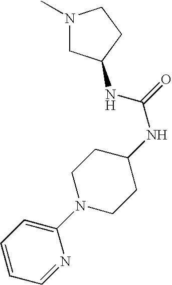 | 736 |
| 10 | 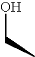 | 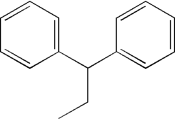 | 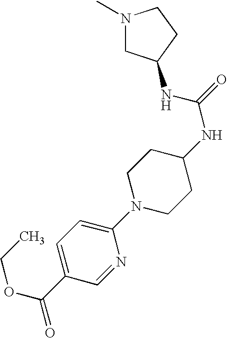 | 808 |
| 11 | 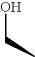 | 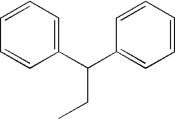 | 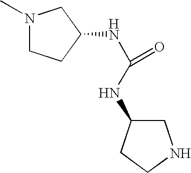 | 645 |
| 12 | 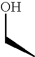 | 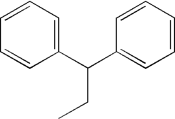 | 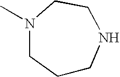 | 547 |
| 13 | 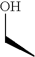 | 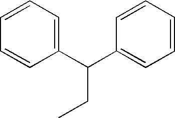 | 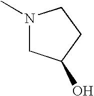 | 533 |
| 14 | 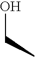 | 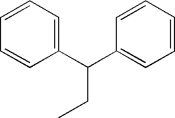 | 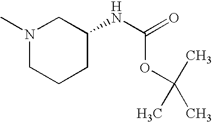 | 647 |
| 15 | 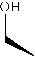 | 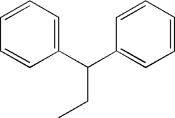 | 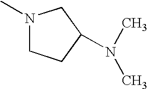 | 561 |
| 16 | 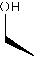 | 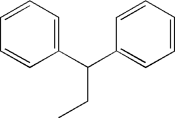 | 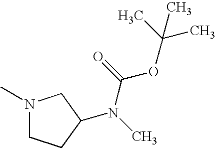 | 646 |
| 17 | 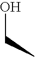 | 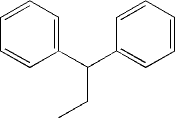 | 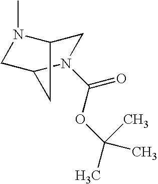 | 645 |
| 18 | 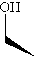 | 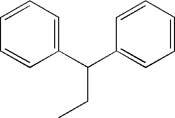 | 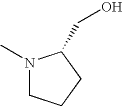 | 548 |
| 19 | 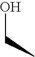 | 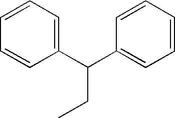 | 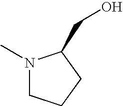 | 546 |
| 20 | 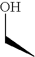 | 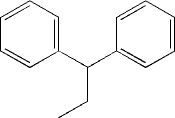 | 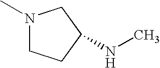 | 546 |
| 21 | 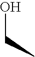 | 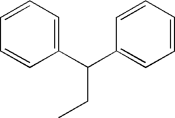 | 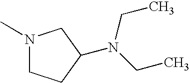 | 589 |
| 22 | 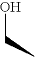 | 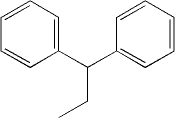 | 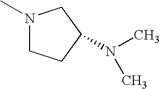 | 560 |
| 23 | 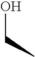 | 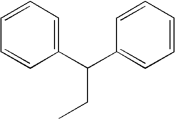 | 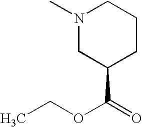 | 603 |
| 24 | 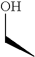 | 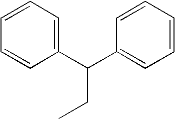 | 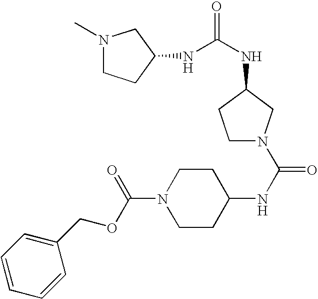 | 452.5(MH +/2) |
| 25 | 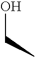 | 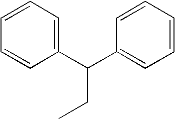 | 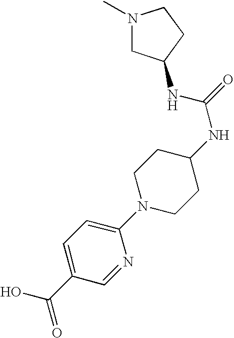 | Nodata |
| 26 | 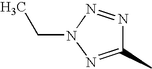 | 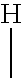 | 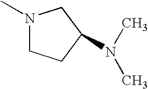 | 446 |
| 27 | 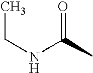 | 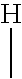 | 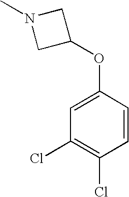 | 524 |
| 28 | 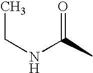 | 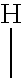 | 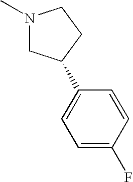 | 472 |
| 29 | 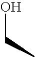 | 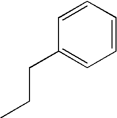 | 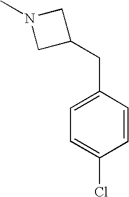 | 551 |
| 30 | 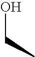 | 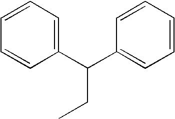 | 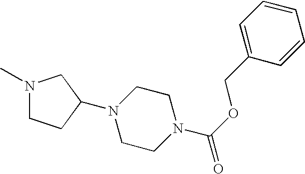 | 736 |
| 31 | 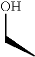 | 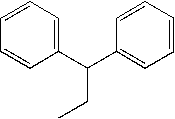 | 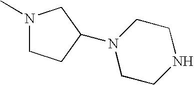 | 601 |
| 32 | 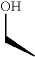 | 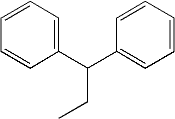 | 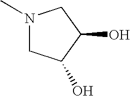 | 549 |
| 33 | 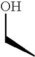 | 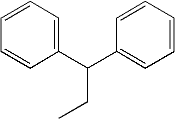 | 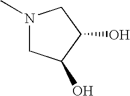 | 549 |
| 34 | 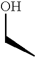 | 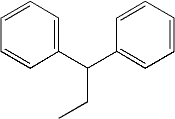 | 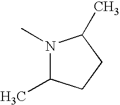 | 545 |
| 35 | 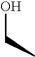 | 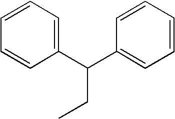 | 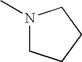 | 517 |
| 36 | 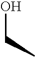 | 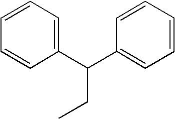 | 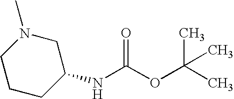 | 646 |
| 37 | 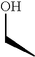 | 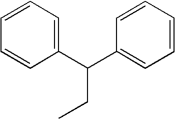 | 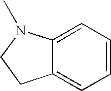 | 565 |
| 38 | 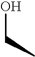 | 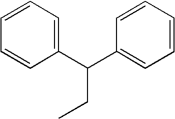 | 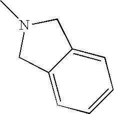 | 565 |
| 39 | 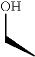 | 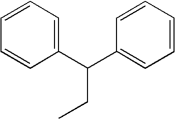 | 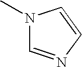 | 514 |
| 40 | 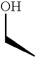 | 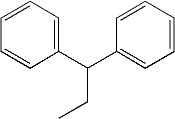 | 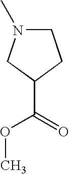 | 589 |
| 41 | 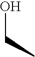 | 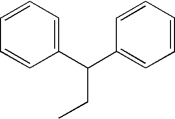 | 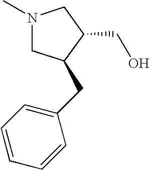 | 637 |
| 42 | 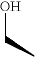 | 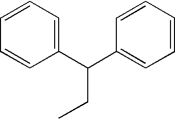 | 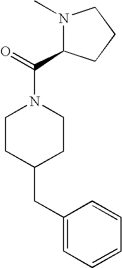 | 718 |
| 43 | 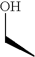 | 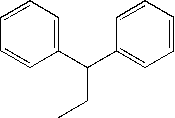 | 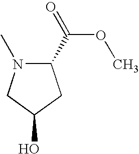 | 591 |
| 44 | 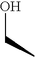 | 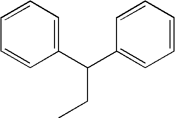 | 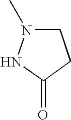 | 532 |
| 45 | 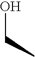 | 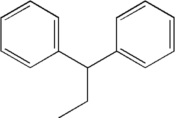 | 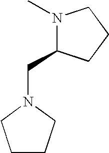 | 600 |
| 46 | 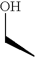 | 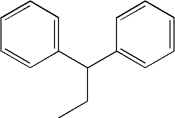 | 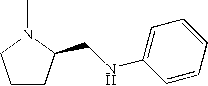 | 622 |
| 47 | 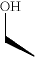 | 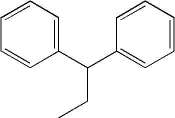 | 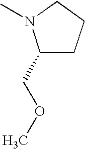 | 561 |
| 48 | 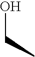 | 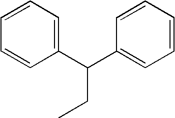 | 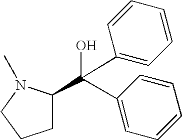 | 699 |
| 49 | 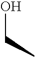 | 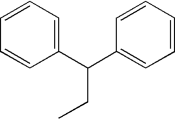 | 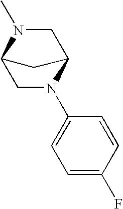 | 638 |
| 50 | 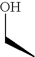 | 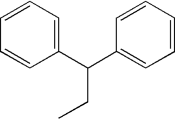 | 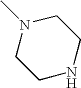 | 532 |
| 51 | 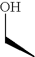 | 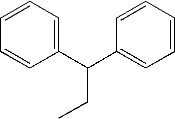 | 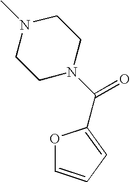 | 626 |
| 52 | 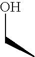 | 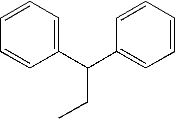 | 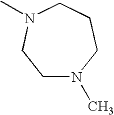 | 560 |
| 53 | 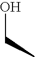 | 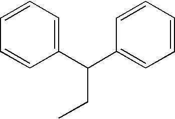 | 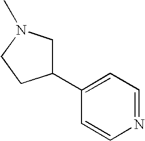 | 594 |
| 54 | 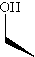 | 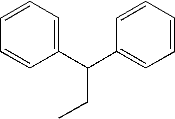 | 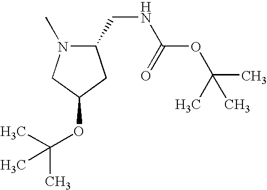 | 718 |
| 55 | 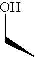 | 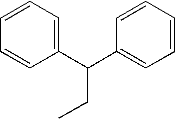 | 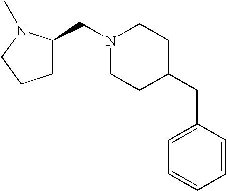 | 704 |
| 56 | 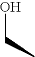 | 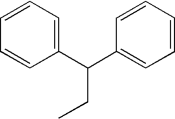 | 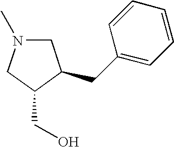 | 637 |
| 57 | 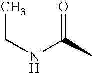 | 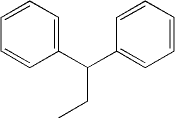 | 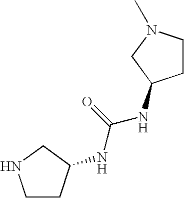 | 685.36 |
| 58 | 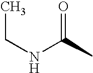 | 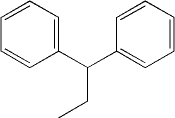 | 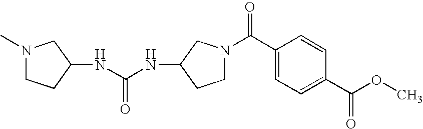 | 847.97 |
| 59 | 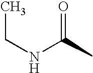 | 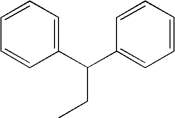 | 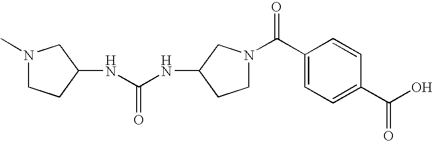 | 833.93 |
| 60 | 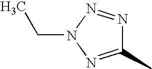 | 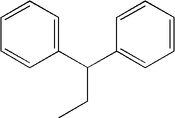 | 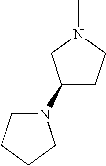 | 652.43 |
| 61 | 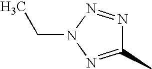 | 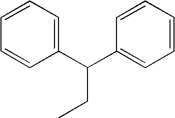 | 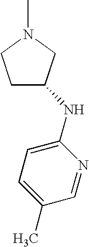 | 689.41 |
| 62 | 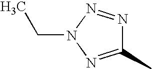 | 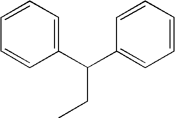 | 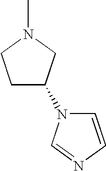 | 649.33 |
| 63 | 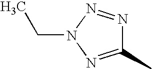 | 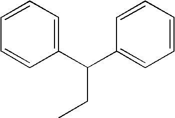 | 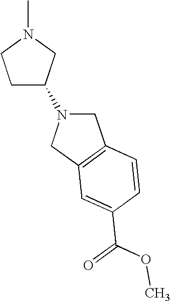 | 757.86 |
| 64 | 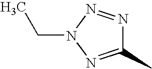 | 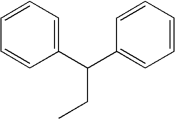 | 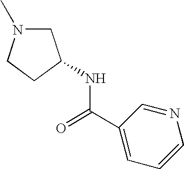 | 702.78 |
| 65 | 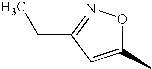 | 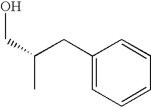 | 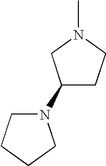 | 605.38 |
| 66 | 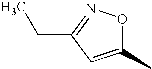 | 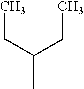 | 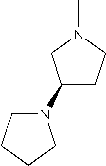 | 541.35 |
| 67 | 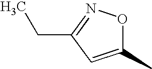 | 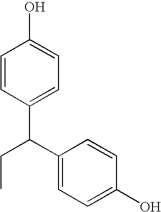 | 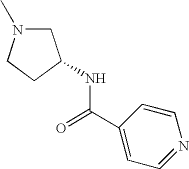 | 734.38 |
| 68 | 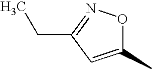 | 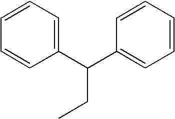 | 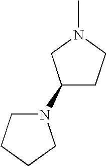 | 651.39 |
| 69 | 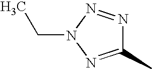 | 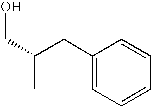 | 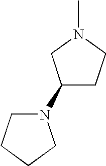 | 606.39 |
| 70 | 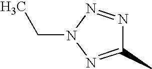 | 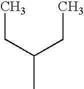 | 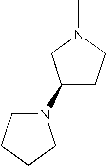 | 542.35 |
| 71 | 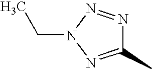 | 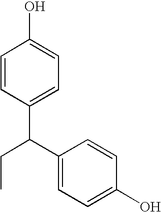 | 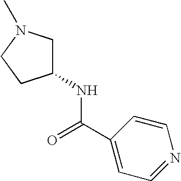 | 735.39 |
| 72 | 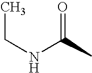 | 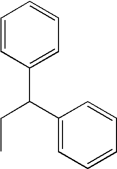 | 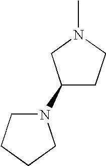 | 627.34 |
| 73 | 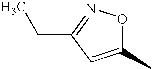 | 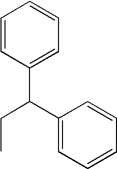 | 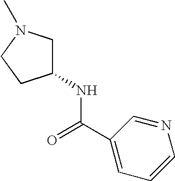 | 702.43 |
| 74 | 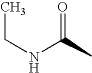 | 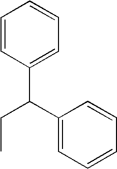 | 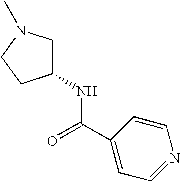 | 677.77 |
| 75 | 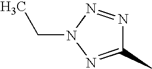 | 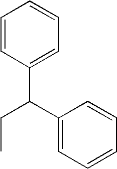 | 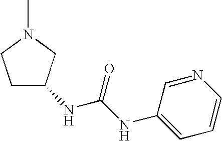 | 718.39 |
| 76 | 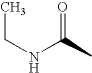 | 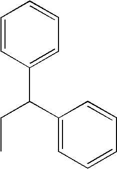 | 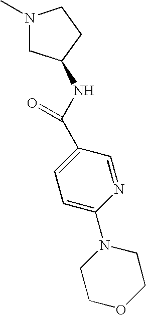 | 762.88 |
| 77 | 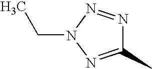 | 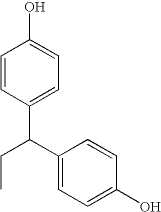 | 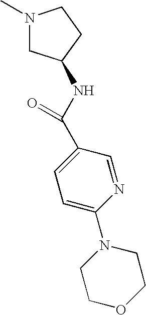 | 820.4 |
| 78 | 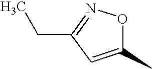 | 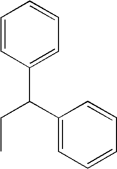 | 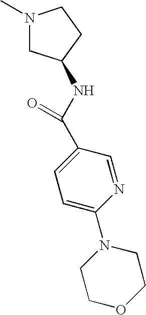 | 788.1 |
| 79 | 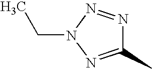 | 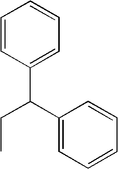 | 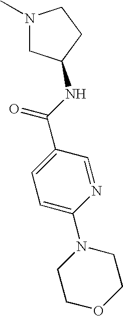 | 789.1 |
| 80 | 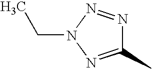 | 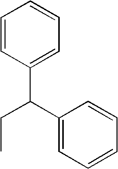 | 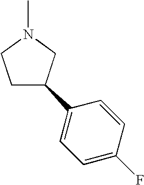 | 677.4 |
| 81 | 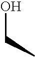 | 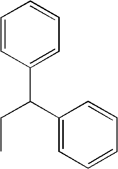 | 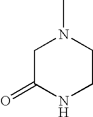 | 546.24 |
| 82 | 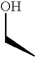 | 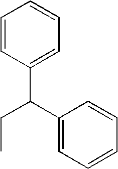 | 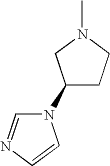 | 583.29 |
| 83 | 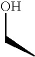 | 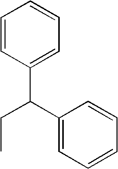 | 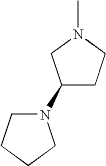 | 586.30 |
| 84 | 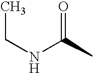 | 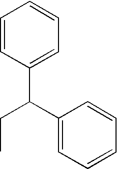 | 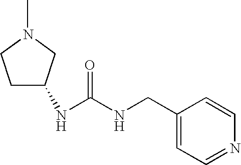 | 707.51 |
| 85 | 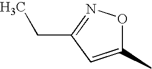 | 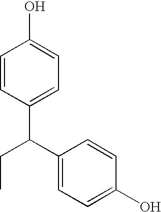 | 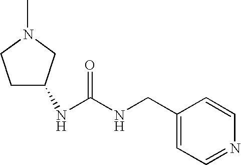 | 763.53 |
| 86 | 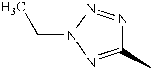 | 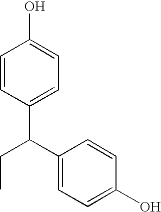 | 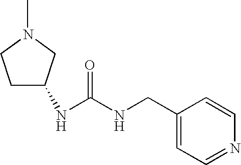 | 764.53 |
| 87 | 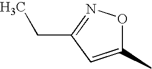 | 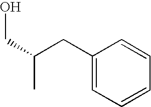 | 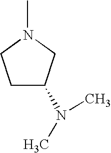 | 579.35 |
| 88 | 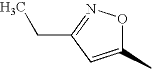 | 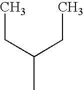 | 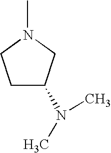 | 515.34 |
| 89 | 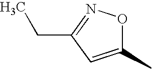 | 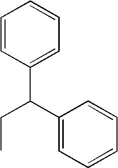 | 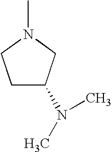 | 625.44 |
| 90 | 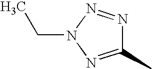 | 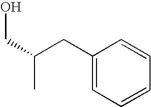 | 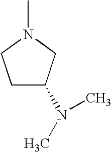 | 580.35 |
| 91 | 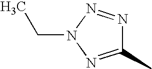 | 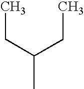 | 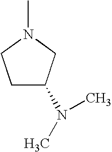 | 516.34 |
| 92 | 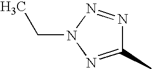 | 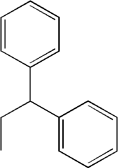 | 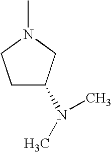 | 626.46 |
| 93 | 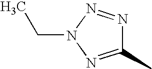 | 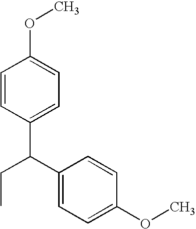 | 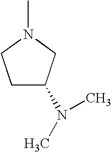 | 686.41 |
| 94 | 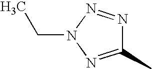 | 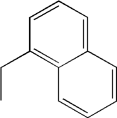 | 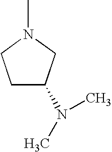 | 586.35 |
| 95 | 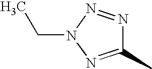 | 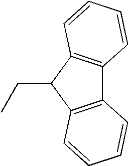 | 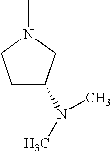 | 624.38 |
| 96 | 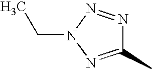 | 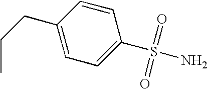 | 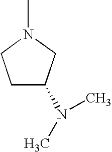 | 629.39 |
| 97 | 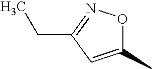 | 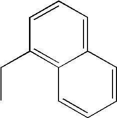 | 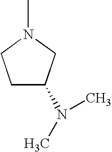 | 585.32 |
| 98 | 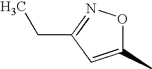 | 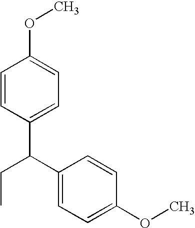 | 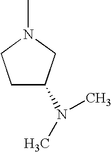 | 685.46 |
| 99 | 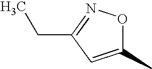 | 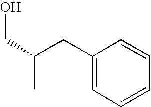 | 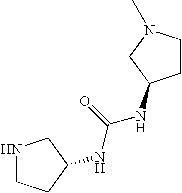 | 663.4 |
| 100 | 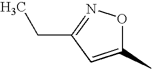 | 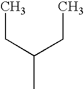 | 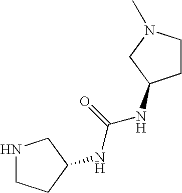 | 599.39 |
| 101 | 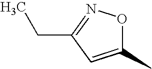 | 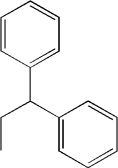 | 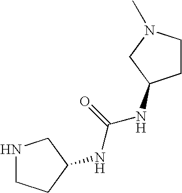 | 709.42 |
| 102 | 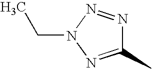 | 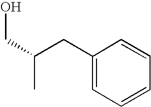 | 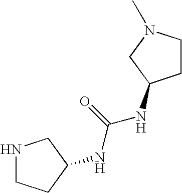 | 664.41 |
| 103 | 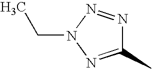 | 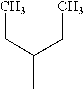 | 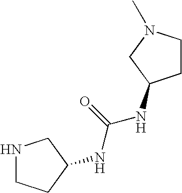 | 600.39 |
| 104 | 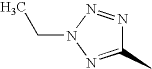 | 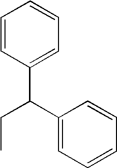 | 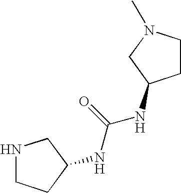 | 710.44 |
| 105 | 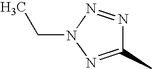 | 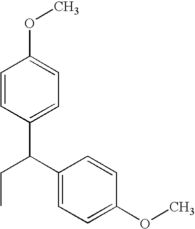 | 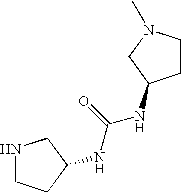 | 769.46 |
| 106 | 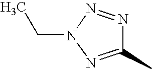 | 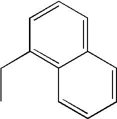 | 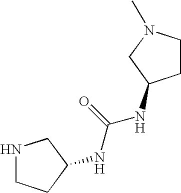 | 670.4 |
| 107 | 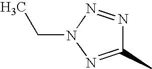 | 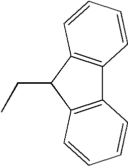 | 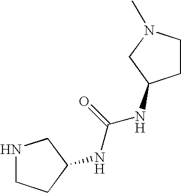 | 708.43 |
| 108 | 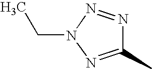 | 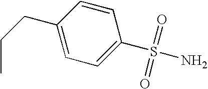 | 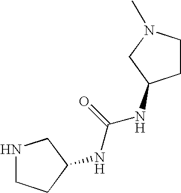 | 713.37 |
| 109 | 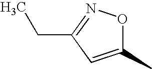 | 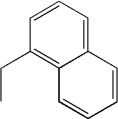 | 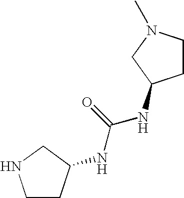 | 669.39 |
| 110 | 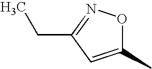 | 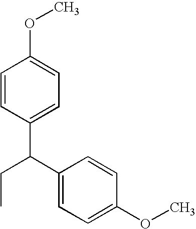 | 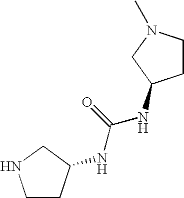 | 769.45 |
| 111 | 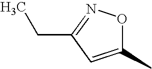 | 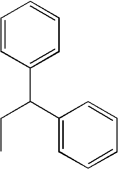 | 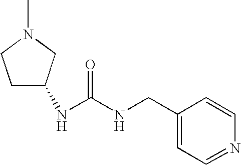 | 731.41 |
| 112 | 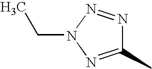 | 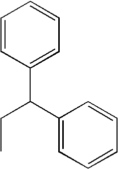 | 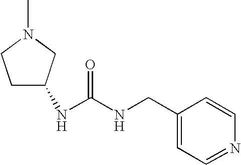 | 732.41 |
| 113 | 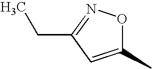 | 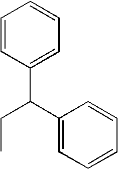 | 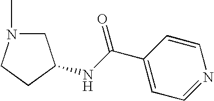 | 702.36 |
| 114 | 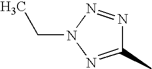 | 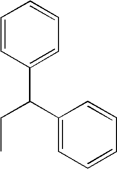 | 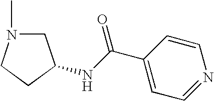 | 703.38 |
| 115 | 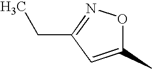 | 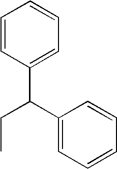 | 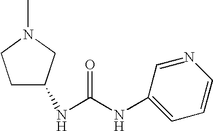 | 717.39 |
| 116 | 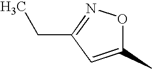 | 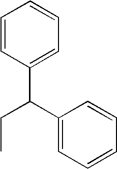 | 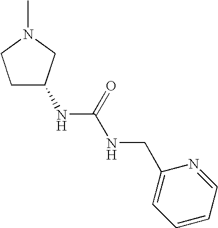 | 731.42 |
| 117 | 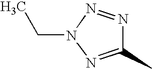 | 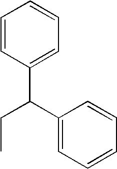 | 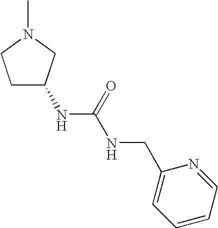 | 732.42 |
| 118 | 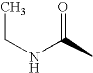 | 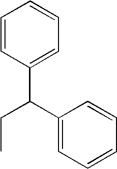 | 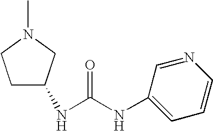 | 693.43 |
| 119 | 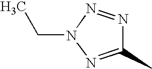 | 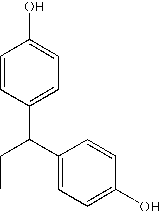 | 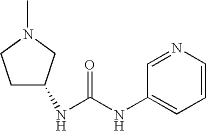 | 750.45 |
| 120 | 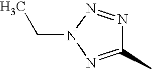 | —H | 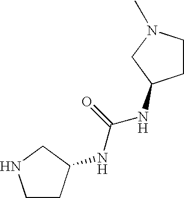 | 530.66 |
| 121 | 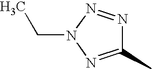 | 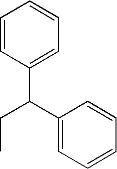 | 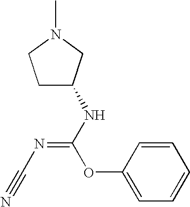 | 724.2 |
| 122 | 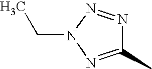 | 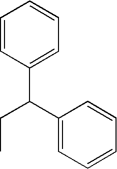 | 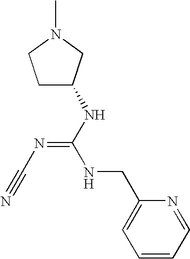 | 757.3 |
| 123 | 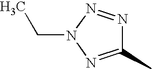 | 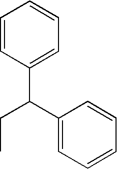 | 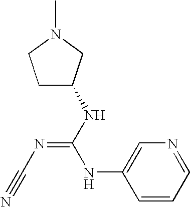 | 742.9 |
| 124 | 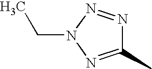 | 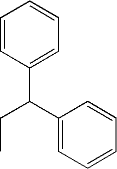 | 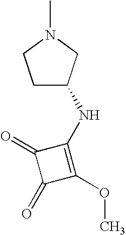 | 708.7 |
| 125 | 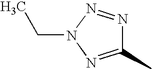 | 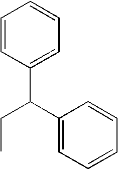 | 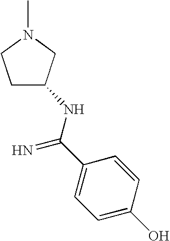 | 717.3 |
| 126 | 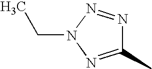 | 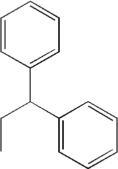 | 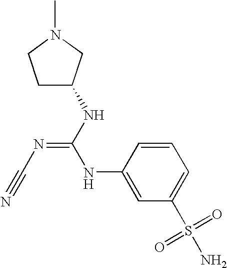 | 820.39 |
| 127 | 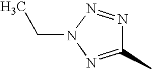 | 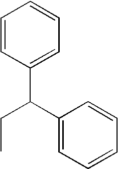 | 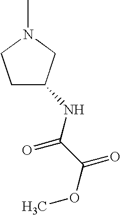 | 684.67 |
| 128 | 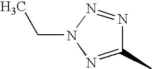 | 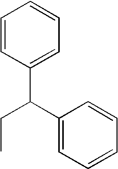 | 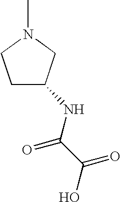 | 670.56 |

### Preparation of Intermediates

Abbreviations used are as follows:

| | |
|---|---|
| 1,1′-carbonyldiimidazole | CDI |
| dichloromethane | DCM |
| diethyl azodicarboxylate | DEAD |
| diisopropylethylamine | DIPEA |
| dimethylformamide | DMF |
| dimethyl sulfoxide | DMSO |
| 1-ethyl-3-(3′-dimethyl- | EDCI |
| aminopropyl)carbodiimide | |
| ethyl acetate | EtOAc |
| ethanol | EtOH |
| high performance liquid | HPLC |
| chromatography | |
| hydrochloric acid | HCl |
| methanol | MeOH |
| magnesium sulfate | MgSO₄ |
| room temperature | RT |
| sodium hydroxide | NaOH |
| tetrahydrofuran | THF |
| trifluoroacetic acid | TFA |

The following intermediates of formula (A)

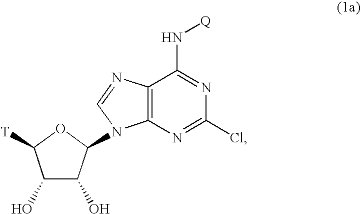

are shown in Table 2 below, their method of preparation being described hereinafter.

TABLE 2

| Intermediate | T | Q | M/s MH⁺ |
|---|---|---|---|
| AA | 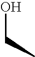 | 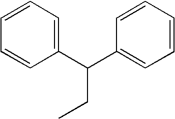 | E-12122-22Roger |
| AB | 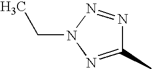 | 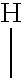 | E-12209-63Andy T |
| AE | 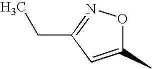 | 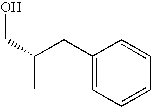 | 501 |
| AF | 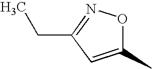 | 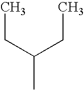 | 437 |
| AG | 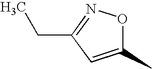 | 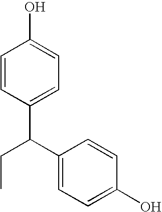 | 579.21 |
| AH | 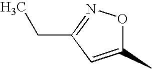 | 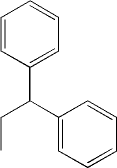 | 547.14 |
| AI | 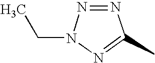 | 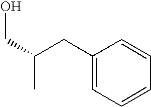 | 502.15 |
| AJ | 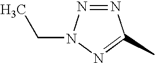 | 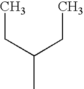 | 438.14 |
| AK | 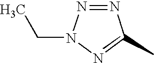 | 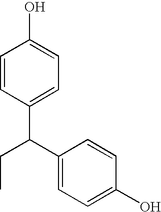 | 580.18 |
| AL | 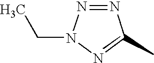 | 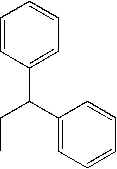 | 548.2 |
| AM | 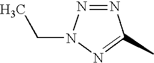 | 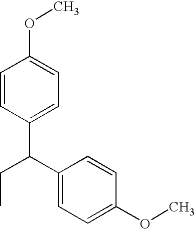 | 608.17 |
| AN | 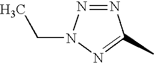 | 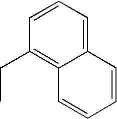 | 508.14 |
| AO | 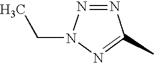 | 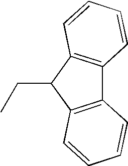 | 546.13 |
| AP | 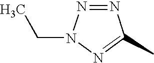 | 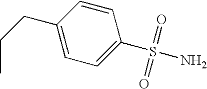 | 551.15 |
| AQ | 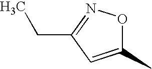 | 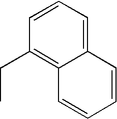 | 507.14 |
| AR | 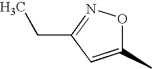 | 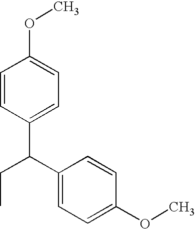 | 607.22 |

### Intermediate AA (2R,3R,4S,5R)-2-[2-Chloro-6-(2,2-diphenyl-ethylamino)-purin-9-yl]-5-hydroxymethyl-tetrahydro-furan-3,4-diol

The title compound is prepared by the procedure of Preparation of Aminopurine-β-D-Ribofuranuronamide Derivatives as Antiinflammatories, Ayres et al., Glaxo Group Limited, UK, PCT Int. Appl., WO 96/02553, 49 pages (1996).

### Intermediate AB (2R,3R,4S,5R)-2-[2-Chloro-6-(2,2-diphenyl-ethylamino)-purin-9-yl]-5-(2-ethyl-2H-tetrazol-5-yl)-tetrahydro-furan-3,4-diol

The title compound is prepared by the procedure of Preparation of 2-(purin-9-yl)-Tetrahydrofuran-3,4-diol Nucleosides as Antiinflammatory Agents and Agonists Against Adenosine Receptors, Cox et al., Glaxo Group Ltd., UK, PCT Int. Appl. WO 98/28319 A1, 118 pages (1998).

### Intermediate AC Acetic acid (2R,3R,4R,5R)-4-acetoxy-2-acetoxymethyl-5-(2-nitro-6-phenethylamino-purin-9-yl)-tetrahydro-furan-3-yl ester

### Step AC1

### Acetic acid (2R,3R,4R,5R)-4-acetoxy-5-acetoxymethyl-2-(6-chloro-2-nitro-purin-9-yl)-tetrahydro-furan-3-yl ester

The title compound is prepared by the procedure of Synthesis and Properties of 2-Nitrosoadenosine, Wanner, Koomen and Gerrit-Jan, Laboratory of Organic Chemistry, Institute of Molecular Chemistry, University of Amsterdam, Amsterdam, Neth., J Chem Soc, Perkin Transactions 1 (16), pp. 1908-1915 (2001).

### Step AC2

### Acetic acid (2R,3R,4R,5R)-4-acetoxy-2-acetoxymethyl-5-(2-nitro-6-phenethylamino-purin-9-yl)-tetrahydro-furan-3-yl ester

To a cooled (0° C.) stirred solution of acetic acid (2R,3R,4R,5R)-4-acetoxy-5-acetoxymethyl-2-(6-chloro-2-nitro-purin-9-yl)-tetrahydro-furan-3-yl ester (Step AC1) (0.3 g, 0.635 mmol), DIPEA (0.101 g, 0.786 mmol) in THF (10 mL) is added phenethylamine (0.087 g, 0.720 mmol). The reaction mixture is allowed to warm to RT whilst stirring continued for 1 hour. The solvent is removed in vacuo and the residue is dissolved in DCM. This organic portion was washed with 1 M HCl and then concentrated in vacuo to yield an oil. Purification by chromatography on silica eluting with DCM:MeOH (99.25:0.75) affords the titled compound as a yellow solid.

### Intermediate AD (3aS,4S,6R,6aR)-6-(6-Amino-2-chloro-purin-9-yl)-2,2-dimethyl-tetrahydro-furo[3,4-d][1,3]dioxole-4-carboxylic acid ethylamide

The title compound is prepared by the procedure of 2-(Arylalkylamino)adenosin-5′-Uronamides: A New Class of Highly Selective Adenosine A2 Receptor Ligands, Hutchison et al., Pharm Div, Ciba-Geigy Corp., Summit, N.J., USA, J Med Chem, Vol. 33 No. 7, pp. 1919-1924 (1990).

### Intermediate AE (2R,3R,4S,5S)-2-[6-((S)-1-Benzyl-2-hydroxy-ethylamino)-2-chloro-purin-9-yl]-5-(3-ethyl-isoxazol-5-yl)-tetrahydro-furan-3,4-diol

### Step AE1

### Acetic acid (2R,3R,4R,5S)-4-acetoxy-2-[6-((S)-1-benzyl-2-hydroxy-ethyl amino)-2-chloro-purin-9-yl]-5-(3-ethyl-isoxazol-5-yl)-tetrahydro-furan-3-yl ester hydrochloride

A mixture comprising acetic acid (2R,3R,4R,5S)-4-acetoxy-2-(2,6-dichloro-purin-9-yl)-5-(3-ethyl-isoxazol-5-yl)-tetrahydro-furan-3-yl ester (WO 99/38877) (1 g, 2.13 mmol), (S)-2-amino-3-phenyl-propan-1-ol (0.321 g, 2.13 mmol) and DIPEA (0.275 g, 2.13 mmol) in DCE (5 mL) is stirred under an inert atmosphere of Argon overnight. After cooling to RT, 1 M HCl is added, the organic portion is separated and concentrated in vacuo to afford the title compound which is used in the next step without further purification. (MH+ 585.1)

### Step AE2

### (2R,3R,4R,5S)-2-[6-((S)-1-Benzyl-2-hydroxy-ethylamino)-2-chloro-purin-9-yl]-5-(3-ethyl-isoxazol-5-yl)-tetrahydro-furan-3,4-diol

A solution of acetic acid (2R,3R,4R,5S)-4-acetoxy-2-[6-((S)-1-benzyl-2-hydroxy-ethyl amino)-2-chloro-purin-9-yl]-5-(3-ethyl-isoxazol-5-yl)-tetrahydro-furan-3-yl ester hydrochloride (Step AC1) (1.194 g, 2.02 mmol) in MeOH/chloroform (4 mL, 3:1 MeOH/chloroform) is treated with saturated potassium carbonate solution (10 mL). After stirring at RT overnight, the reaction mixture is diluted with DCM/water and the organic portion is separated. The organic portion is concentrated in vacuo to afford the title compound. (MH+ 501)

### Intermediates AF-AH

These intermediates namely,
(2R,3R,4S,5S)-2-[2-Chloro-6-(1-ethyl-propylamino)-purin-9-yl]-5-(3-ethyl-isoxazol-5-yl)-tetrahydro-furan-3,4-diol (Intermediate AF);
(2R,3R,4S,5S)-2-{6-[2,2-bis-(4-hydroxy-phenyl)-ethylamino]-2-chloro-purin-9-yl}-5-(3-ethyl-isoxazol-5-yl)-tetrahydro-furan-3,4-diol (Intermediate AG); and
(2R,3R,4S,5S)-2-[2-Chloro-6-(2,2-diphenyl-ethylamino)-purin-9-yl]-5-(3-ethyl-isoxazol-5-yl)-tetrahydro-furan-3,4-diol (Intermediate AH),
   are prepared analogously to Intermediate AE by replacing (S)-2-amino-3-phenyl-propan-1-ol with the appropriate amine.

### Intermediate AI (2R,3R,4S,5R)-2-[6-((S)-1-Benzyl-2-hydroxy-ethylamino)-2-chloro-purin-9-yl]-5-(2-ethyl-2H-tetrazol-5-yl)-tetrahydro-furan-3,4-diol

### Step AI1

### Acetic acid (2R,3R,4R,5R)-4-acetoxy-2-[6-((S)-1-benzyl-2-hydroxy-ethyl amino)-2-chloro-purin-9-yl]-5-(2-ethyl-2H-tetrazol-5-yl)-tetrahydro-furan-3-yl ester

The title compound is prepared analogously to acetic acid (2R,3R,4R,5S)-4-acetoxy-2-[6-((S)-1-benzyl-2-hydroxy-ethyl amino)-2-chloro-purin-9-yl]-5-(3-ethyl-isoxazol-5-yl)-tetrahydro-furan-3-yl ester hydrochloride (Step AE1) by replacing acetic acid (2R,3R,4R,5S)-4-acetoxy-2-(2,6-dichloro-purin-9-yl)-5-(3-ethyl-isoxazol-5-yl)-tetrahydro-furan-3-yl ester (WO 99/38877) with acetic acid (2R,3R,4R,5R)-4-acetoxy-2-(2,6-dichloro-purin-9-yl)-5-(2-ethyl-2H-tetrazol-5-yl)-tetrahydro-furan-3-yl ester (WO 98/28319).

### Step AI2

### (2R,3R,4S,5R)-2-[6-((S)-1-Benzyl-2-hydroxy-ethylamino)-2-chloro-purin-9-yl]-5-(2-ethyl-2H-tetrazol-5-yl)-tetrahydro-furan-3,4-diol

The title compound is prepared from acetic acid (2R,3R,4R,5R)-4-acetoxy-2-[6-((S)-1-benzyl-2-hydroxy-ethylamino)-2-chloro-purin-9-yl]-5-(2-ethyl-2H-tetrazol-5-yl)-tetrahydro-furan-3-yl ester (Step AI1) analogously to (2R,3R,4S,5S)-2-[6-((S)-1-benzyl-2-hydroxy-ethylamino)-2-chloro-purin-9-yl]-5-(3-ethyl-isoxazol-5-yl)-tetrahydro-furan-3,4-diol.

### Intermediates AJ-AP

These intermediates namely,
(2R,3R,4S,5R)-2-[2-Chloro-6-(1-ethyl-propylamino)-purin-9-yl]-5-(2-ethyl-2H-tetrazol-5-yl)-tetrahydro-furan-3,4-diol (Intermediate AJ);
(2R,3R,4S,5R)-2-{6-[2,2-bis-(4-Hydroxy-phenyl)-ethylamino]-2-chloro-purin-9-yl}-5-(2-ethyl-2H-tetrazol-5-yl)-tetrahydro-furan-3,4-diol (Intermediate AK);
(2R,3R,4S,5R)-2-[2-Chloro-6-(2,2-diphenyl-ethylamino)-purin-9-yl]-5-(2-ethyl-2H-tetrazol-5-yl)-tetrahydro-furan-3,4-diol (Intermediate AL);
(2R,3R,4S,5R)-2-(6-[2,2-bis-(4-Methoxy-phenyl)-ethylamino]-2-chloro-purin-9-yl)-5-(2-ethyl-2H-tetrazol-5-yl)-tetrahydro-furan-3,4-diol (Intermediate AM);
(2R,3R,4S,5R)-2-{2-Chloro-6-[(naphthalen-1-ylmethyl)-amino]-purin-9-yl}-5-(2-ethyl-2H-tetrazol-5-yl)-tetrahydro-furan-3,4-diol (Intermediate AN);
(2R,3R,4S,5R)-2-{2-Chloro-6-[(9H-fluoren-9-ylmethyl)-amino]-purin-9-yl}-5-(2-ethyl-2H-tetrazol-5-yl)-tetrahydro-furan-3,4-diol (Intermediate AO); and
4-(2-{2-Chloro-9-[(2R,3R,4S,5R)-5-(2-ethyl-2H-tetrazol-5-yl)-3,4-dihydroxy-tetrahydro-furan-2-yl]-9H-purin-6-ylamino}-ethyl)-benzenesulfonamide (Intermediate AP),
   are prepared analogously to Intermediate AI by replacing (S)-2-amino-3-phenyl-propan-1-ol with the appropriate amine.

### Intermediates AQ-AR

These compounds namely,
(2R,3R,4S,5S)-2-{2-Chloro-6-[(naphthalen-1-ylmethyl)-amino]-purin-9-yl}-5-(3-ethyl-isoxazol-5-yl)-tetrahydro-furan-3,4-diol (Intermediate AQ); and
(2R,3R,4S,5S)-2-{6-[2,2-bis-(4-methoxy-phenyl)-ethylamino]-2-chloro-purin-9-yl}-5-(3-ethyl-isoxazol-5-yl)-tetrahydro-furan-3,4-diol (Intermediate AR),
   are prepared analogously to Intermediate AE by replacing (S)-2-amino-3-phenyl-propan-1-ol with the appropriate amine.

The following intermediates were used in the synthesis of some of the final compounds listed in Table 1:

### Intermediate BA 4-(4-Fluoro-phenyl)-piperidine

4-(4-Fluoro-phenyl)-1,2,3,6-tetrahydro-pyridine chloride (20 g, 93.7 mmol) is dissolved in anhydrous MeOH (200 mL) under an inert atmosphere of argon. The solution is then treated with 10% palladium on carbon (1 g). The reaction mixture is purged with argon and placed under an atmosphere of hydrogen overnight. The mixture is then filtered through Celite™ filter material and the catalyst is washed with MeOH. The filtrate and washings are evaporated to dryness and the resultant residue is partitioned between 2 M NaOH and diethyl ether. The layers are separated and the aqueous is extracted with two further portions of ether. The organic portions are combined, washed with brine, dried (MgSO₄) and concentrated in vacuo to yield the titled compound as a yellow oil.

### Intermediate BB Imidazole-1-carboxylic acid (3,4,5,6-tetrahydro-2H-[1,2′]bipyridinyl-4-yl)-amide

A stirred solution of CDI (1.1 g, 6.77 mmol) in DCM (100 mL) is treated with 3,4,5,6-tetrahydro-2H-[1,2′]bipyridinyl-4-ylamine (WO 99/65895; EP 21973) (1 g, 5.64 mmol in 50 mL of DCM) added dropwise over 30 minutes. The reaction mixture is stirred at RT for 15 minutes to yield the titled compound as a 10 mg/mL solution in DCM. The compound is used in solution in subsequent reactions. This solution consists of the imidazole-urea Intermediate BB together with variable amounts of the corresponding isocyanate and imidazole which result from reversible thermal elimination of imidazole under the reaction conditions. This solution is used in the subsequent steps since the imidazole-urea intermediate and isocyanate intermediate are equally suitable as precursors to ureas.

### Intermediate BC 4-[(Imidazole-1-carbonyl)-amino]-3,4,5,6-tetrahydro-2H-[1,2′]bipyridinyl-5′-carboxylic acid ethyl ester

### Step BC1

### 4-Carbamoyl-3,4,5,6-tetrahydro-2H-[1,2′]bipyridinyl-5′-carboxylic acid ethyl ester

A stirred suspension comprising 6-chloro-nicotinic acid ethyl ester (1.86 g, 10.0 mmol), piperidine-4-carboxamide (1.54 g, 12.0 mmol) and DIPEA (2.1 mL, 12 mmol) in DMSO (7 mL) is heated to 90° C. for 2 hours. MeOH (8 mL) is then added as the reaction mixture cools and the resulting precipitate is filtered, washed with water followed by ether and dried in vacuo (45° C.) to yield the titled compound as a white powder.

### Step BC2

### 4-Amino-3,4,5,6-tetrahydro-2H-[1,2′]bipyridinyl-5′-carboxylic acid ethyl ester

A solution comprising 4-carbamoyl-3,4,5,6-tetrahydro-2H-[1,2′]bipyridinyl-5′-carboxylic acid ethyl ester (2.04 g, 7.36 mmol) and bis(trifluoroacetoxy) iodobenzene (3.80 g, 8.83 mmol) in acetonitrile (13 mL) is treated with water (5 mL) and heated to 65° C. for 30 hours. The solvent is partially removed in vacuo and the resulting solution is acidified to pH 1 using 12 M HCl. The solution is extracted with EtOAc and this organic portion is discarded. The aqueous portion is basified to pH 8-9 using 2 M potassium carbonate solution and then extracted with EtOAc then DCM. The combined organic portions are washed with brine, dried (Na₂SO₄) and concentrated in vacuo. The resulting residue is triturated with ether followed by ether/EtOAc (1:1, 5×0.7 mL) and dried in vacuo to yield the titled product as an off-white solid.

### Step BC3

### 4-[(Imidazole-1-carbonyl)-amino]-3,4,5,6-tetrahydro-2H-[1,2′]bipyridinyl-5′-carboxylic acid ethyl ester

To a solution of 4-amino-3,4,5,6-tetrahydro-2H-[1,2′]bipyridinyl-5′-carboxylic acid ethyl ester (0.103 g, 0.414 mmol) and triethylamine (0.12 mL, 0.828 mmol) in DCM (4.14 mL) is added CDI (0.073 g, 0.455 mmol). The reaction mixture is stirred at RT for 2 hours to afford the titled compound as a 0.1 M solution in DCM. The compound is used in solution in subsequent reactions. This solution consists of the imidazole-urea Intermediate BC together with variable amounts of the corresponding isocyanate and imidazole which result from reversible thermal elimination of imidazole under the reaction conditions. This solution is used in the subsequent steps since the imidazole-urea intermediate and isocyanate intermediate are equally suitable as precursors to ureas.

### Intermediate BD 1,3-di(R)-Pyrrolidin-3-yl-urea

### Step BD1

### 1,3-bis-((R)-1-Benzyl-pyrrolidin-3-yl)-urea

A solution comprising (R)-1-benzyl-pyrrolidin-3-ylamine (5.0 g, 28.4 mmol) in DCM (10 mL) is treated with CDI (2.3 g, 14.2 mmol) and the reaction mixture is stirred at RT for 48 hours. The solvent is removed in vacuo and the resulting residue is dissolved in EtOAc. This portion is washed with water followed by brine, dried (MgSO₄) and concentrated in vacuo to yield the titled compound as pale orange solid. MS [ESI+]: m/z: 379.2 (MH⁺).

### Step BD2

### 1,3-di(R)-Pyrrolidin-3-yl-urea

To a solution of 1,3-bis-((R)-1-benzyl-pyrrolidin-3-yl)-urea (5.34 g, 14.1 mmol) in EtOH (80 mL) under an inert atmosphere of argon is added palladium hydroxide on carbon (1.07 g). The reaction mixture is purged with argon and placed under an atmosphere of hydrogen for 2 days after which time, the mixture is filtered and the catalyst washed with EtOH. The organic portions are combined and concentrated in vacuo to yield the titled compound as a white solid. MS [ESI+]: m/z: 199.1 (MH⁺).

### Intermediate BE 4-Pyrrolidin-3-yl-piperazine-1-carboxylic acid benzyl ester

This compound is prepared using the procedure described in International Patent Application WO 2002/0445652.

### Intermediate BF ((3R,4R)-4-Benzyl-pyrrolidin-3-yl)-methanol hydrochloride

A solution comprising (3R,4R)-3-benzyl-4-hydroxymethyl-pyrrolidine-1-carboxylic acid tert-butyl ester (0.2 g, 0.69 mmol) in dioxane (1 mL) is treated with 4 M HCl-dioxane (3.44 mL, 13.7 mmol) and allowed to stir at RT overnight. The solvent is removed in vacuo to yield the titled compound. MS (ESI+) m/z 192.1 (MH⁺).

### Intermediate BG (3-Methylamino-propyl)-carbamic acid tert-butyl ester

The title compound is prepared by the procedure of The Selective Reaction of Primary Carbonyl Imidazole Containing Compounds: Selective Amide and Carbamate Synthesis. Rannard and Davis, Org Lett, Vol. 2, No. 14, pp. 2117-2120 (2000).

### Intermediate BH 4-Benzyl-1-(R)-1-pyrrolidin-2-ylmethyl-piperidine

### Step BH1

### (R)-2-(4-Benzyl-piperidine-1-carbonyl)-pyrrolidine-1-carboxylic acid benzyl ester

A solution of Z-D-proline (10.0 g, 40.1 mmol), 4-benzylpiperidine (7.0 g, 40.1 mmol), hydroxybenztriazole (5.96 g, 44 mmol) and EDCI (8.46 g, 44 mmol) in DCM (100 mL) is stirred at RT for 16 hours. The solvent was removed in vacuo and the residue is taken up in EtOAc (200 mL). The EtOAc solution is washed with 1 N HCl, 1 M sodium carbonate, water and brine and then dried (Na₂SO₄). The solvent is removed in vacuo to yield the titled compound. MS [ESI+]: m/z: 407 (MH⁺).

### Step BH2

### (4-Benzyl-piperidin-1-yl)-(R)-pyrrolidin-2-yl-methanone

To a solution of (R)-2-(4-benzyl-piperidine-1-carbonyl)-pyrrolidine-1-carboxylic acid benzyl ester (6.62 g, 16.3 mmol) in MeOH (130 mL) is added palladium hydroxide on carbon (0.5 g) and the mixture is placed under an atmosphere of hydrogen until the reaction has gone to completion. The mixture is filtered and the filtrate is concentrated in vacuo to yield the titled compound. MS [ESI+]: m/z: 273 (MH⁺).

### Step BH3

### 4-Benzyl-1-(R)-1-pyrrolidin-2-ylmethyl-piperidine

(4-Benzyl-piperidin-1-yl)-(R)-pyrrolidin-2-yl-methanone (4.25 g, 15.6 mmol) is added dropwise to a suspension of lithium aluminum hydride (0.89 g, 23.5 mmol) in THF (30 mL) at RT. The reaction mixture is heated to reflux for 16 hours and then allowed to cool and poured onto ice. The solution is adjusted to pH 10 using aqueous sodium hydroxide. The product is extracted into EtOAc and the organic portions are combined, washed with water, brine, dried (Na₂SO₄) and concentrated in vacuo to yield the titled. MS [ESI+]: m/z: 259 (MH⁺).

### Intermediate BI ((2S,4R)-4-tert-Butoxy-pyrrolidin-2-ylmethyl)-carbamic acid tert-butyl ester

### Step BI1

### (2S,4R)-4-tert-Butoxy-2-hydroxymethyl-pyrrolidine-1-carboxylic acid benzyl ester

A mixture comprising (2S,4R)-4-tert-butoxy-pyrrolidine-1,2-dicarboxylic acid 1-benzyl ester (23.5 g, 72.4 mmol) and triethylamine (10.1 mL, 72.4 mmol) in THF (210 mL) is cooled to 0° C. and treated with ethyl chloroformate (7.04 mL, 72.4 mmol) over 10 minutes. After 40 minutes, the resulting white solid is filtered and washed with THF. The filtrate is cooled to 0° C. and sodium borohydride (9.04 g, 231.7 mmol) is added. MeOH (50 mL) is then added dropwise over 45 minutes. The reaction mixture is stirred for 15 minutes at RT and then treated with 1 M HCl (520 mL). After stirring for 1.5 hours, the reaction mixture is extracted 3 times with DCM. The combined organic layers are dried (Na₂SO₄) and concentrated in vacuo. The resulting crude is purified by flash chromatography on silica gel eluting with hexane:EtOAc (7:3) to afford the titled compound as a colourless oil.

### Step BI2

### (2S,4R)-4-teit-Butoxy-2-(1,3-dioxo-1,3-dihydro-isoindol-2-ylmethyl)-pyrrolidine-1-carboxylic acid benzyl ester

A cooled suspension comprising (2S,4R)-4-tert-butoxy-2-hydroxymethyl-pyrrolidine-1-carboxylic acid benzyl ester (19.2 g, 62.5 mmol), phthalimide (9.2 g, 62.5 mmol) and triphenylphosphine (6.7 g, 62.5 mmol) in THF (260 mL) is carefully treated dropwise with DEAD (3.7 mL, 62.46 mmol). After stirring at RT for 2 hours, further portions of phthalimide (0.92 g, 6.2 mmol), triphenylphosphine (0.67 g, 6.2 mmol) and DEAD (0.37 mL, 6.2 mmol) are added. The resulting red solution is stirred at RT overnight and the solvent is removed in vacuo. The resulting crude is purified by chromatography on silica eluting with EtOAc:hexane (7:9) to yield the titled compound as a yellow oil.

### Step BI3

### (2S,4R)-2-Aminomethyl-4-tert-butoxy-pyrrolidine-1-carboxylic acid benzyl ester

(2S,4R)-4-tert-Butoxy-2-(1,3-dioxo-1,3-dihydro-isoindol-2-ylmethyl)-pyrrolidine-1-carboxylic acid benzyl ester (12.8 g, 29.3 mmol) is dissolved in EtOH (165 mL) and hydrazine monohydrate (14.2 mL, 322 mmol) is added. After stirring at RT, a white suspension forms. The reaction mixture is heated to reflux for 30 minutes. After cooling to RT, the suspension is filtered off and the solid washed 4 times with EtOH. The filtrate is concentrated in vacuo and dried under high vacuum at 40° C. to give the titled compound which is used without further purification in the next step.

### Step BI4

### (2S,4R)-4-tert-Butoxy-2-(tert-butoxycarbonylamino-methyl)-pyrrolidine-1-carboxylic acid benzyl ester

A mixture of crude (2S,4R)-2-aminomethyl-4-tert-butoxy-pyrrolidine-1-carboxylic acid benzyl ester (12.3 g, 29.3 mmol) and Boc anhydride (6.6 g, 30.2 mmol) in DCM (120 mL) is stirred at RT overnight. The reaction mixture is washed successively with 1 M HCl, 10% sodium carbonate solution and brine. The aqueous layers are extracted twice with DCM. The combined organic portions are dried (Na₂SO₄) and concentrated in vacuo. The resulting crude is purified by chromatography on silica eluting with hexane:EtOAc (9:1 increasing to 7:3) followed by trituration with hexane:diisopropyl ether 9:1 to give the titled compound as a white solid.

### Step BI5

### ((2S,4R)-4-tert-Butoxy-pyrrolidin-2-ylmethyl)-carbamic acid tert-butyl ester

A solution of (2S,4R)-4-tert-butoxy-2-(tert-butoxycarbonylamino-methyl)-pyrrolidine-1-carboxylic acid benzyl ester (34.7 g, 82.9 mmol) in THF (500 mL) is hydrogenated over catalytic Pd/C to give the title compound after filtration, evaporation and drying as a pale yellow oil.

### Intermediate C (2S,3S,4R,5R)-5-[2-Chloro-6-(2,2-diphenyl-ethylamino)-purin-9-yl]-3,4-dihydroxy-tetrahydro-furan-2-carboxylic acid ethylamide

The title compound can be prepared by the procedure of Gregson, Michael; Ayres, Barry Edward; Ewan, George Blanch; Ellis, Frank; Knight, John. Preparation of diaminopurinylribofuranuronamide derivatives as antiinflammatories. (WO 94/17090)

### Intermediate D 4,4′-(2-Aminoethylidene)bis-phenol

The preparation of this compound is described in (WO 2001/036375).

### Intermediate E (R)-[1,3′]Bipyrrolidinyl

### Step E1

### (R)-1′-Benzyl-[1,3′]bipyrrolidinyl

An ice-cooled solution of 2,5-dimethoxytetrahydrofuran (19.11 mL, 0.147 mol) and 6 M sulphuric acid (37.2 mL) in THF (200 mL) is treated dropwise with (R)-(1)-benzyl-3-aminopyrrolidine (10 g, 0.057 mol) in THF (150 mL) and sodium borohydride pellets (8.62 g, 0.227 mol) simultaneously, ensuring the temperature remains below 10° C. The reaction mixture is allowed to warm to RT and water (10 mL) is added to aid dissolution of the NaOH pellets. After stirring at RT for 12 days, the mixture is cooled with the use on an ice-bath and water is added (500 mL). The solution is basified by addition of NaOH pellets (pH<10) and then filtered under vacuum. The filtrate is extracted with diethyl ether and DCM and the organic portions are combined and concentrated in vacuo. The crude residue is sonicated in diethyl ether and filtered under vacuum. The filtrate is reduced in vacuo again and the resulting crude is dissolved in acetonitrile (8 mL) and purified by reverse phase column chromatography (Isolute™ C18, 0-100% acetonitrile in water −0.1% TFA) to yield the title product.

### Step E2

### (R)-[1,3′]Bipyrrolidinyl

A solution of (R)-1′-benzyl-[1,3′]bipyrrolidinyl (0.517 g, 2.24 mmol) in MeOH (25 mL) under an atmosphere of Argon is treated with palladium hydroxide on carbon (0.1 g). The reaction mixture is placed under an atmosphere of hydrogen and stirred at RT overnight and then filtered through Celite™. The filtrate is concentrated in vacuo to yield the title product as a dark orange oil.

### Intermediate F (5-Methyl-pyridin-2-yl)-(R)-pyrrolidin-3-yl-amine

### Step F1

### 6-((R)-1-Benzyl-pyrrolidin-3-ylamino)-nicotinonitrile

A solution of 2-chloro-5-cyano-pyridine (0.5 g, 3.6 mmol) in DMF (10 mL) is treated with 3-R-amino-1-N-benzyl-pyrrolidine (0.638 g, 3.6 mmol) and DIPEA (0.467 mL, 3.6 mmol) and stirred at 50° C. for 6 hours. The reaction mixture is diluted with water and extracted with EtOAc (2×50 ml). The combined organic extracts are concentrated in vacuo to afford the title compound as an oil. MS [ESI+]: m/z: 279.1 (MH⁺).

### Step F2

### (5-Methyl-pyridin-2-yl)-(R)-pyrrolidin-3-yl-amine

The title compound is prepared analogously to (4-benzyl-piperidin-1-yl)-(R)-pyrrolidin-2-yl-methanone (Intermediate BH2).

### Intermediate G (R)—N-Pyrrolidin-3-yl-nicotinamide

### Step G1

### (R)-3-[(Pyridine-4-carbonyl)-amino]-pyrrolidine-1-carboxylic acid tert-butyl ester

A cooled (0° C.) stirred solution of (R)-3-amino-pyrrolidine-1-carboxylic acid tert-butyl ester (1.0 g, 5.36 mmol) and TEA (1.5 mL, 11.0 mmol) in THF (10 mL) is treated dropwise over 1 minute with pyridine-3 carbonyl chloride hydrochloride (0.935 g, 5.25 mmol). After 5 minutes, the reaction mixture is allowed to warm to RT and stirred overnight. The resulting mixture is diluted with EtOAc and washed twice with saturated sodium bicarbonate solution followed by brine. The organic portion is dried (MgSO₄) and concentrated in vacuo. The crude product is purified by recrystallisation from EtOAc/iso-hexane to afford the title product. (MH+ 292.2)

### Step G2

### (R)—N-Pyrrolidin-3-yl-nicotinamide

A solution of (R)-3-[(pyridine-4-carbonyl)-amino]-pyrrolidine-1-carboxylic acid tert-butyl ester (1.38 g, 4.74 mmol) in MeOH (2 mL) is treated with 2 M HCl (2 mL) and left to stand at RT overnight. The resulting mixture is diluted with MeOH and concentrated in vacuo. Co-evaporation of the residue with EtOAc/MeOH followed by neat EtOAc afford the title compound as a white solid. (MH+ 192.1)

### Intermediate H(R)-2-Pyrrolidin-3-yl-2,3-dihydro-1H-isoindole-5-carboxylic acid methyl ester

### Step H1

### 2-((R)-1-Benzyl-pyrrolidin-3-yl)-2,3-dihydro-1H-isoindole-5-carboxylic acid methyl ester

To a solution of 3-(R)-amino-1-benzylpyrrolidone (0.5 g, 2.8 mmol) in acetonitrile (10 mL) under an inert atmosphere of Argon is added DIPEA (1 mL) followed by 3,4-bis-bromomethyl-benzoic acid methyl ester (1.0 g, 2.9 mmol). The resulting mixture is stirred at RT overnight and then diluted with DCM. The reaction is quenched with water and the organic portion is separated and concentrated in vacuo to afford the title compound as an orange oil. (MH+ 337.2)

### Step H2

### (R)-2-Pyrrolidin-3-yl-2,3-dihydro-1H-isoindole-5-carboxylic acid methyl ester

The title compound is prepared analogously to (4-benzyl-piperidin-1-yl)-(R)-pyrrolidin-2-yl-methanone (Intermediate BH2).

### Intermediate I (R)—N-Pyrrolidin-3-yl-isonicotinamide

### Step I1

### (R)-3-[(Pyridine-4-carbonyl)-amino]-pyrrolidine-1-carboxylic acid tert-butyl ester

A cooled (0° C.) stirred solution of (R)-3-amino-pyrrolidine-1-carboxylic acid tert-butyl ester (1.0 g, 5.36 mmol) and TEA (1.5 mL, 11.0 mmol) in THF (10 mL) is treated dropwise over 1 minute with pyridine-4 carbonyl chloride hydrochloride (0.935 g, 5.25 mmol). After 5 minutes, the reaction mixture is allowed to warm to RT and stirred overnight. The resulting mixture is diluted with EtOAc and washed twice with saturated sodium bicarbonate solution followed by brine. The organic portion is dried (MgSO₄) and concentrated in vacuo. The crude product is purified by recrystallisation from EtOAc/iso-hexane to afford the title product. (MH+ 292)

### Step I2

### (R)—N-Pyrrolidin-3-yl-isonicotinamide

A solution of (R)-3-[(pyridine-4-carbonyl)-amino]-pyrrolidine-1-carboxylic acid tert-butyl ester (1.38 g, 4.74 mmol) in MeOH (6 mL) is treated with 2 M HCl (5 mL) and left to stand at RT overnight. The resulting mixture is diluted with MeOH and added to 12 mL of Dowex resin (50Wx2-200). After 30 minutes, the resin is washed with water until neutral and then further washed off with MeOH and 2% ammonia. The solvent is removed in vacuo to afford the title compound as a crystalline solid. (MH+ 192)

### Intermediate J (2S,3S,4R,5R)-5-[2-Chloro-6-(2,2-diphenyl-ethylamino)-purin-9-yl]-3,4-dihydroxy-tetrahydro-furan-2-carboxylic acid ethylamide

The preparation of this compound is described in (WO 94/17090).

### Intermediate K (R)-3-(4-Fluoro-phenyl)-pyrrolidine

Racemic 3-(4-fluoro-phenyl)-pyrrolidine (696 g, 3.7 mol) is suspended in EtOH (11 L) and heated to 55-60° C. to give a solution, whereupon a solution of (+)-di-O,O-p-tolyl tartaric acid (814 g, 2.1 mol) in EtOH (3 L) is added over 20 minutes. The solution is cooled to 0° C. over 4 hours and stirred overnight to give an off-white suspension which is washed with two portions of cold EtOH (2×450 mL). The resulting solid is dissolved in EtOH (9 L) at 60° C. and then cooled over 4 hours to 22° C. The resulting suspension is filtered and washed with two portions of EtOH (2×300 mL). The re-crystallisation was repeated twice more using EtOH (6.5 L) to afford the title product.

### PREPARATION OF SPECIFIC EXAMPLES

### Example 1

### (2R,3R,4S,5R)-2-{6-(2,2-Diphenyl-ethylamino)-2-[4-(4-fluoro-phenyl)-piperidin-1-yl]-purin-9-yl}-5-hydroxymethyl-tetrahydro-furan-3,4-diol

To a stirred solution of (2R,3R,4S,5R)-2-[2-chloro-6-(2,2-diphenyl-ethylamino)-purin-9-yl]-5-hydroxymethyl-tetrahydro-furan-3,4-diol (0.15 g, 0.31 mmol) in DMSO (2 mL) is added DIPEA (0.12 g, 1.24 mmol) and 4-(4-fluoro-phenyl)-piperidine (0.16 g, 0.94 mmol). The reaction mixture is stirred at 140° C. overnight and then allowed to cool to room temperature. The mixture is diluted with EtOAc and washed with water (4×10 mL). The organic portion is dried (MgSO₄) and concentrated in vacuo. The crude residue is purified by C-18 reverse phase column chromatography eluting with acetonitrile:water (gradient of 0-100% acetonitrile) to afford the titled compound as a brown solid.

### Examples 2-5

These compounds namely,
(2R,3R,4S,5R)-2-{6-(2,2-diphenyl-ethylamino)-2-[(R)-3-(4-fluoro-phenyl)-pyrrolidin-1-yl]-purin-9-yl}-5-hydroxymethyl-tetrahydro-furan-3,4-diol trifluoroacetate (Example 2);
{(S)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid tert-butyl ester trifluoroacetate (Example 3);
(2R,3R,4S,5R)-2-{6-(2,2-diphenyl-ethylamino)-2-[3-(4-methoxy-phenyl)-pyrrolidin-1-yl]-purin-9-yl}-5-hydroxymethyl-tetrahydro-furan-3,4-diol trifluoroacetate (Example 4); and
{(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid tert-butyl ester trifluoroacetate (Example 5),
   are prepared by an analogous procedure to Example 1 by replacing 4-(4-fluoro-phenyl)-piperidine with the appropriate amine.

### Example 6

### (2R,3R,4S,5R)-2-[2-((S)-3-Amino-pyrrolidin-1-yl)-6-(2,2-diphenyl-ethylamino)-purin-9-yl]-5-hydroxymethyl-tetrahydro-furan-3,4-diol trifluoroacetate

A stirred solution of {(S)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid teit-butyl ester trifluoroacetate (0.5 g, 0.79 mmol) in DCM (2 mL) is treated with TFA (1.5 mL) and stirred for 30 minutes. The solvent is removed in vacuo and the resulting oil is dissolved in MeOH and concentrated in vacuo again. This process is repeated twice to yield the titled compound.

### Example 7

### (2R,3R,4S,5R)-2-[2-((R)-3-Amino-pyrrolidin-1-yl)-6-(2,2-diphenyl-ethylamino)-purin-9-yl]-5-hydroxymethyl-tetrahydro-furan-3,4-diol trifluoroacetate

The titled compound is prepared analogously to Example 6 by replacing {(S)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid tert-butyl ester trifluoroacetate with {(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid tedt-butyl ester trifluoroacetate.

### Example 8

### (2R,3R,4S,5R)-2-[2-((R)-3-Amino-piperidin-1-yl)-6-(2,2-diphenyl-ethylamino)-purin-9-yl]-5-hydroxymethyl-tetrahydro-furan-3,4-diol trifluoroacetate

### Step 1

### {(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-piperidin-3-yl}-carbamic acid tert-butyl ester

The titled compound is prepared analogously to Example 1 by replacing 4-(4-fluoro-phenyl)-piperidine with (R)-piperidin-3-yl-carbamic acid tert-butyl ester.

### Step 2

### (2R,3R,4S,5R)-2-[2-((R)-3-Amino-piperidin-1-yl)-6-(2,2-diphenyl-ethylamino)-purin-9-yl]-5-hydroxymethyl-tetrahydro-furan-3,4-diol trifluoroacetate

The titled compound is prepared analogously to Example 6 by replacing {(S)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid tert-butyl ester trifluoroacetate with {(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-piperidin-3-yl}-carbamic acid tert-butyl ester.

### Example 9

### 1-{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidin-3-yl}-3-(3,4,5,6-tetrahydro-2H-[1,2′]bipyridinyl-4-yl)-urea trifluoroacetate

A stirred solution of (2R,3R,4S,5R)-2-[2-((R)-3-amino-pyrrolidin-1-yl)-6-(2,2-diphenyl-ethylamino)-purin-9-yl]-5-hydroxymethyl-tetrahydro-furan-3,4-diol trifluoroacetate (0.03 g, 0.05 mmol) in toluene/isopropyl alcohol (6 mL of 2:1 toluene:isopropyl alcohol) is treated with triethylamine (0.0094 g, 0.09 mmol) followed by imidazole-1-carboxylic acid (3,4,5,6-tetrahydro-2H-[1,2′]bipyridinyl-4-yl)-amide (2.09 mL of a 10 mg/mL solution in DCM, 0.08 mmol). After stirring at room temperature for two days, the solvent is removed under reduced pressure and the product is purified by C-18 reverse phase column chromatography eluting with acetonitrile:water (0.1% TFA) (gradient of 0-100% acetonitrile) to afford the titled compound.

### Example 10

### 4-(3-(R)-1-[9-((2R,3R,4S,5R)-3,4-Dihydroxy-5-hydromethyl-tetrahydro-furan-2-yl)-6-(2,2-diphenyl-ethylamino)-9H-purin-2-yl]-pyrrolindin-3-yl)-ureido)-3,4,5,6-tetrahydro-2H-[1,2′]bipyridinyl-5′-carboxylic acid ethyl ester trifluoroacetate

The titled compound is prepared by the same procedure as Example 9 by replacing the imidazole-1-carboxylic acid (3,4,5,6-tetrahydro-2H-[1,2′]bipyridinyl-4-yl)-amide with 4-[(imidazole-1-carbonyl)-amino]-3,4,5,6-tetrahydro-2H-[1,2′]bipyridinyl-5′-carboxylic acid ethyl ester.

### Example 11

### 1-{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidin-3-yl}-3-(R)-pyrrolidin-3-yl-urea trifluoroacetate

To a stirred solution of (2R,3R,4S,5R)-2-[2-chloro-6-(2,2-diphenyl-ethylamino)-purin-9-yl]-5-hydroxymethyl-tetrahydro-furan-3,4-diol (0.05 g, 0.1 mmol) and sodium iodide (0.016 g, 0.1 mmol) in acetonitrile:NMP (1.0 mL of a 1:1 solution) is added 1,3-di(R)-pyrrolidin-3-yl-urea (0.041 g, 0.2 mmol) and DIPEA (0.05 mL, 0.26 mmol). The reaction mixture is heated to 160° C. for 30 minutes in a microwave. Purification by C-18 reverse phase column chromatography eluting with acetonitrile:water (0.1% TFA) (gradient of 0-100% acetonitrile) affords the titled compound.

### Examples 12-27

These compounds namely,
(2R,3R,4S,5R)-2-[2-[1,4]diazepan-1-yl-6-(2,2-diphenyl-ethylamino)-purin-9-yl]-5-hydroxymethyl-tetrahydro-furan-3,4-diol trifluoroacetate (Example 12);
(2R,3R,4S,5R)-2-[6-(2,2-diphenyl-ethylamino)-2-((R)-3-hydroxy-pyrrolidin-1-yl)-purin-9-yl]-5-hydroxymethyl-tetrahydro-furan-3,4-diol (Example 13);
{(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-piperidin-3-yl}-carbamic acid tert-butyl ester trifluoroacetate (Example 14);
(2R,3R,4S,5R)-2-[2-(3-dimethylamino-pyrrolidin-1-yl)-6-(2,2-diphenyl-ethylamino)-purin-9-yl]-5-hydroxymethyl-tetrahydro-furan-3,4-diol trifluoroacetate (Example 15);
{1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-methyl-carbamic acid tert-butyl ester (Example 16);
5-[9-((2R,3R,4S,5R)-3,4-dihydroxy-5-hydroxymethyl-tetrahydro-furan-2-yl)-6-(2,2-diphenyl-ethylamino)-9H-purin-2-yl]-2,5-diaza-bicyclo[2.2.1]heptane-2-carboxylic acid tert-butyl ester trifluoroacetate (Example 17);
(2R,3R,4S,5R)-2-[6-(2,2-diphenyl-ethylamino)-2-((S)-2-hydroxymethyl-pyrrolidin-1-yl)-purin-9-yl]-5-hydroxymethyl-tetrahydro-furan-3,4-diol trifluoroacetate (Example 18);
(2R,3R,4S,5R)-2-[6-(2,2-diphenyl-ethylamino)-2-((R)-2-hydroxymethyl-pyrrolidin-1-yl)-purin-9-yl]-5-hydroxymethyl-tetrahydro-furan-3,4-diol (Example 19);
(2R,3R,4S,5R)-2-[6-(2,2-diphenyl-ethylamino)-2-((R)-3-methylamino-pyrrolidin-1-yl)-purin-9-yl]-5-hydroxymethyl-tetrahydro-furan-3,4-diol trifluoroacetate (Example 20);
(2R,3R,4S,5R)-2-[2-(3-diethylamino-pyrrolidin-1-yl)-6-(2,2-diphenyl-ethylamino)-purin-9-yl]-5-hydroxymethyl-tetrahydro-furan-3,4-diol trifluoroacetate (Example 21);
(2R,3R,4S,5R)-2-[2-((R)-3-dimethylamino-pyrrolidin-1-yl)-6-(2,2-diphenyl-ethylamino)-purin-9-yl]-5-hydroxymethyl-tetrahydro-furan-3,4-diol trifluoroacetate (Example 22); and
(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-piperidine-3-carboxylic acid ethyl ester trifluoroacetate (Example 23),
   are prepared analogously to Example 11 by replacing 1,3-di(R)-pyrrolidin-3-yl-urea with the appropriate amine.

### Example 24

### 4-{[(R)-3-(3-{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidine-3-yl}-ureido)-pyrrolidine-1-carbonyl]-amino}-piperidine-1-carboxylic acid benzyl ester trifluoroacetate

A stirred solution 1-{(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-3-(R)-pyrrolidin-3-yl-urea trifluoroacetate (0.015 g, 0.02 mmol) in THF (2 mL) is treated with benzyl-4-isocyanatotetrahydro-1(2H)-pyridine carboxylate (0.01 g, 0.08 mmol) and triethylamine (0.004 g, 0.04 mmol). The reaction mixture is stirred at RT overnight and then the solvent is removed in vacuo. Purification by C-18 reverse phase column chromatography eluting with acetonitrile:water (0.1% TFA) (gradient of 0-100% acetonitrile) to afford the titled compound.

### Example 25

### 4-(3-{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidin-3-yl}-ureido)-3,4,5,6-tetrahydro-2H-[1,2′]bipyridinyl-5′-carboxylic acid trifluoroacetate

4-(3-{(R)-1-[9-((2R,3R,4S,5R)-3,4-dihydroxy-5-hydromethyl-tetrahydro-furan-2-yl)-6-(2,2-diphenyl-ethylamino)-9H-purin-2-yl]-pyrrolindin-3-yl}-ureido)-3,4,5,6-tetrahydro-2H-[1,2′]bipyridinyl-5′-carboxylic acid ethyl ester trifluoroacetate (0.015 g, 0.02 mmol) is dissolved in methanol (2 mL) and then treated with lithium hydroxide (0.004 g, 0.33 mmol). The reaction mixture is stirred at RT overnight and the solvent removed in vacuo. Purification by C-18 reverse phase column chromatography eluting first with water and then with methanol yields the titled compound.

### Example 26

### (2R,3R,4S,5R)-2-[6-Amino-2-((R)-3-dimethylamino-pyrrolidin-1-yl)-purin-9-yl]-5-(2-ethyl-2H-tetrazol-5-yl)-tetrahydro-furan-3,4-diol trifluoroacetate

The titled compound is prepared by the same procedure as Example 11 by replacing (2R,3R,4S,5R)-2-[2-chloro-6-(2,2-diphenyl-ethylamino)-purin-9-yl]-5-hydroxymethyl-tetrahydro-furan-3,4-diol with (2R,3R,4S,5R)-2-[2-chloro-6-(2,2-diphenyl-ethylamino)-purin-9-yl]-5-(2-ethyl-2H-tetrazol-5-yl)-tetrahydro-furan-3,4-diol and by replacing 1,3-di(R)-pyrrolidin-3-yl-urea with dimethyl-(S)-pyrrolidin-3-yl-amine.

### Example 27

### (2R,3R,4S,5R)-5-{6-Amino-2-[3-(3,4-dichloro-phenoxy)-azetidin-1-yl]-purin-9-yl}-3,4-dihydroxy-tetrahydro-furan-2-carboxylic acid ethylamide trifluoroacetate

### Step 1

### (3aS,4S,6R,6aR)-6-{6-Amino-2-[3-(3,4-dichloro-phenoxy)-azetidin-1-yl]-purin-9-yl}-2,2-dimethyl-tetrahydro-furo[3,4-a][1,3]dioxole-4-carboxylic acid ethylamide

(3aS,4S,6R,6aR)-6-(6-Amino-2-chloro-purin-9-yl)-2,2-dimethyl-tetrahydro-furo[3,4-d][1,3]dioxole-4-carboxylic acid ethylamide (0.1 g, 0.261 mmol) and 3-(3,4-dichloro-phenoxy)-azetidine (WO 2003/077907) (0.128 g, 0.574 mmol) are treated with NMP (0.1 mL) and heated to 165° C. overnight. Purification by chromatography on silica eluting with EtOAc:hexane (1:1) followed by MeOH/EtOAc (1:10) affords the titled compound as a yellow oil.

### Step 2

### (2R,3R,4S,5R)-5-{6-Amino-2-[3-(3,4-dichloro-phenoxy)-azetidin-1-yl]-purin-9-yl}-3,4-dihydroxy-tetrahydro-furan-2-carboxylic acid ethylamide trifluoroacetate

A solution of (3aS,4S,6R,6aR)-6-{6-amino-2-[3-(3,4-dichloro-phenoxy)-azetidin-1-yl]-7H-pyrrolo[3,2-c]pyrimidin-7-yl}-2,2-dimethyl-tetrahydro-furo[3,4-a][1,3]dioxole-4-carboxylic acid ethylamide (0.016 g, 0.028 mmol) in dioxane (5 mL) is treated with HCl (5 mL of a 2 M aqueous solution). The reaction mixture is stirred at RT for 24 hours. The solvent is removed in vacuo and purification by C-18 reverse phase column chromatography eluting with acetonitrile:water (0.1% TFA) (gradient of 0-100% acetonitrile) yields the titled compound.

### Example 28

### (2R,3R,4S,5R)-5-{6-Amino-2-[(R)-3-(4-fluoro-phenyl)-pyrrolidin-1-yl]-purin-9-yl}-3,4-dihydroxy-tetrahydro-furan-2-carboxylic acid ethylamide trifluoroacetate

The titled compound is prepared by the same procedure as Example 33 by replacing 3-(3,4-dichloro-phenoxy)-azetidine with (R)-3-(4-fluoro-phenyl)-pyrrolidine.

### Example 29

### (2R,3R,4S,5R)-2-{2-[3-(4-Chloro-benzyl)-azetidin-1-yl]-6-phenethylamino-purin-9-yl}-5-hydroxymethyl-tetrahydro-furan-3,4-diol

### Step 1

### Acetic acid (2R,3R,4S,5R)-3,4-diacetoxy-5-{2-[3-(4-chloro-benzyl)-azetidin-1-yl]-6-phenethylamino-purin-9-yl}-tetrahydro-furan-2-ylmethyl ester

The titled compound is prepared by the same procedure as Example 1 by replacing (2R,3R,4S,5R)-2-[2-chloro-6-(2,2-diphenyl-ethylamino)-purin-9-yl]-5-hydroxymethyl-tetrahydro-furan-3,4-diol with acetic acid (2R,3R,4S,5R)-4-acetoxy-2-acetoxymethyl-5-(2-nitro-6-phenethylamino-purin-9-yl)-tetrahydro-furan-3-yl ester and by replacing 4-(4-fluoro-phenyl)-piperidine with 3-(4-chloro-benzyl)-azetidine (WO 2003/077907).

### Step 2

### (2R,3R,4S,5R)-2-{2-[3-(4-Chloro-benzyl)-azetidin-1-yl]-6-phenethylamino-purin-9-yl}-5-hydroxymethyl-tetrahydro-furan-3,4-diol

A solution of acetic acid (2R,3R,4S,5R)-3,4-diacetoxy-5-{2-[3-(4-chloro-benzyl)-azetidin-1-yl]-6-phenethylamino-purin-9-yl}-tetrahydro-furan-2-yi methyl ester (0.0025 g, 0.0004 mmol) in MeOH (1 mL) is treated with potassium carbonate (0.002 g, 0.014 mmol). The reaction mixture is concentrated in vacuo and purification by C-18 reverse phase column chromatography eluting with acetonitrile:water (gradient of 0-100% acetonitrile) to afford the titled compound.

### Example 30

### 4-{1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidin-3-yl}-piperazine-1-carboxylic acid benzyl ester trifluoroacetate

The titled compound is prepared analogously to Example 1 by replacing 4-(4-fluoro-phenyl)-piperidine with 4-pyrrolidin-3-yl-piperazine-1-carboxylic acid benzyl ester.

### Example 31

### (2R,3R,4S,5R)-2-[6-(2,2-Diphenyl-ethylamino)-2-(3-piperazin-1-yl-pyrrolidin-1-yl)-purin-9-yl]-5-hydroxymethyl-tetrahydro-furan-3,4-diol

To a solution comprising 4-{1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-piperazine-1-carboxylic acid benzyl ester trifluoroacetate (0.02 g, 23.6 μmol) in ethanol (2 mL) is added palladium on carbon (10% w/w) (0.005 g) and the reaction mixture is placed under an atmosphere of hydrogen. The reaction mixture is stirred at RT for 19 hours and filtered through Celite™. The filtrate is concentrated in vacuo to yield the titled compound as a solid.

### Examples 32-56

These compounds namely,
(3R,4R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidine-3,4-diol trifluoroacetate (Example 32);
(3S,4S)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidine-3,4-diol trifluoroacetate (Example 33);
(2R,3R,4S,5R)-2-[2-(2,5-Dimethyl-pyrrolidin-1-yl)-6-(2,2-diphenyl-ethylamino)-purin-9-yl]-5-hydroxymethyl-tetrahydro-furan-3,4-diol trifluoroacetate (Example 34);
(2R,3R,4S,5R)-2-[6-(2,2-Diphenyl-ethylamino)-2-pyrrolidin-1-yl-purin-9-yl]-5-hydroxymethyl-tetrahydro-furan-3,4-diol trifluoroacetate (Example 35);
{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-piperidin-3-yl}-carbamic acid tert-butyl ester trifluoroacetate (Example 36);
(2R,3R,4S,5R)-2-[2-(2,3-Dihydro-indol-1-yl)-6-(2,2-diphenyl-ethylamino)-purin-9-yl]-5-hydroxymethyl-tetrahydro-furan-3,4-diol trifluoroacetate (Example 37);
(2R,3R,4S,5R)-2-[2-(1,3-Dihydro-isoindol-2-yl)-6-(2,2-diphenyl-ethylamino)-purin-9-yl]-5-hydroxymethyl-tetrahydro-furan-3,4-diol trifluoroacetate (Example 38);
(2R,3R,4S,5R)-2-[6-(2,2-Diphenyl-ethylamino)-2-imidazol-1-yl-purin-9-yl]-5-hydroxymethyl-tetrahydro-furan-3,4-diol trifluoroacetate (Example 39);
1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidine-3-carboxylic acid methyl ester trifluoroacetate (Example 40);
(2R,3R,4S,5R)-2-[2-((3R,4R)-3-Benzyl-4-hydroxymethyl-pyrrolidin-1-yl)-6-(2,2-diphenyl-ethylamino)-purin-9-yl]-5-hydroxymethyl-tetrahydro-furan-3,4-diol trifluoroacetate (Example 41);
(4-Benzyl-piperidin-1-yl)-{(S)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-2-yl}-methanone trifluoroacetate (Example 42);
(2S,4R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-4-hydroxy-pyrrolidine-2-carboxylic acid methyl ester trifluoroacetate (Example 43);
1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrazolidin-3-one trifluoroacetate (Example 44);
(2R,3R,4S,5R)-2-[6-(2,2-Diphenyl-ethylamino)-2-((S)-2-pyrrolidin-1-ylmethyl-pyrrolidin-1-yl)-purin-9-yl]-5-hydroxymethyl-tetrahydro-furan-3,4-diol trifluoroacetate (Example 45);
(2R,3R,4S,5R)-2-[6-(2,2-Diphenyl-ethylamino)-2-((R)-2-phenylaminomethyl-pyrrolidin-1-yl)-purin-9-yl]-5-hydroxymethyl-tetrahydro-furan-3,4-diol trifluoroacetate (Example 46);
(2R,3R,4S,5R)-2-[6-(2,2-Diphenyl-ethylamino)-2-((R)-2-methoxymethyl-pyrrolidin-1-yl)-purin-9-yl]-5-hydroxymethyl-tetrahydro-furan-3,4-diol trifluoroacetate (Example 47);
(2R,3R,4S,5R)-2-{6-(2,2-Diphenyl-ethylamino)-2-[(R)-2-(hydroxy-diphenyl-methyl)-pyrrolidin-1-yl]-purin-9-yl}-5-hydroxymethyl-tetrahydro-furan-3,4-diol trifluoroacetate (Example 48);
(2R,3R,4S,5R)-2-{6-(2,2-Diphenyl-ethylamino)-2-[(1S,4S)-5-(4-fluoro-phenyl)-2,5-diaza-bicyclo[2.2.1]hept-2-yl]-purin-9-yl}-5-hydroxymethyl-tetrahydro-furan-3,4-diol trifluoroacetate (Example 49);
(2R,3R,4S,5R)-2-[6-(2,2-Diphenyl-ethylamino)-2-piperazin-1-yl-purin-9-yl]-5-hydroxymethyl-tetrahydro-furan-3,4-diol trifluoroacetate (Example 50);
{4-[9-((2R,3R,4S,5R)-3,4-Dihydroxy-5-hydroxymethyl-tetrahydro-furan-2-yl)-6-(2,2-diphenyl-ethylamino)-9H-purin-2-yl]-piperazin-1-yl}-furan-2-yl-methanone trifluoroacetate (Example 51);
(2R,3R,4S,5R)-2-[6-(2,2-Diphenyl-ethylamino)-2-(4-methyl-[1,4]diazepan-1-yl)-purin-9-yl]-5-hydroxymethyl-tetrahydro-furan-3,4-diol trifluoroacetate (Example 52);
(2R,3R,4S,5R)-2-[6-(2,2-Diphenyl-ethylamino)-2-(3-pyridin-4-yl-pyrrolidin-1-yl)-purin-9-yl]-5-hydroxymethyl-tetrahydro-furan-3,4-diol trifluoroacetate (Example 53);
{(2S,4R)-4-tert-Butoxy-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-2-ylmethyl}-carbamic acid tert-butyl ester trifluoroacetate (Example 54);
(2R,3R,4S,5R)-2-[2-[(R)-2-(4-Benzyl-piperidin-1-ylmethyl)-pyrrolidin-1-yl]-6-(2,2-diphenyl-ethylamino)-purin-9-yl]-5-hydroxymethyl-tetrahydro-furan-3,4-diol trifluoroacetate (Example 55); and
(2R,3R,4S,5R)-2-[2-((3S,4S)-3-Benzyl-4-hydroxymethyl-pyrrolidin-1-yl)-6-(2,2-diphenyl-ethylamino)-purin-9-yl]-5-hydroxymethyl-tetrahydro-furan-3,4-diol trifluoroacetate (Example 56),
   are prepared analogously to Example 11 by replacing 1,3-di(R)-pyrrolidin-3-yl-urea with the appropriate amine. The amines that are used to prepare these examples are described herein or are commercially-available or prepared by standard methods.

### Example 57

### (2S,3S,4R,5R)-5-{6-(2,2-Diphenyl-ethylamino)-2-[(R)-3-((R)-3-pyrrolidin-3-ylureido)-pyrrolidin-1-yl]-purin-9-yl}-3,4-dihydroxy-tetrahydro-furan-2-carboxylic acid ethylamide hydrochloride

The title compound is prepared analogously to Example 1 by replacing (2R,3R,4S,5R)-2-[2-chloro-6-(2,2-diphenyl-ethylamino)-purin-9-yl]-5-hydroxymethyl-tetrahydro-furan-3,4-diol with (2S,3S,4R,5R)-5-[2-chloro-6-(2,2-diphenyl-ethylamino)-purin-9-yl]-3,4-dihydroxy-tetrahydro-furan-2-carboxylic acid ethylamide (Intermediate C) and by replacing 4-(4-fluoro-phenyl)-piperidine (Intermediate BA) with 1,3-di(R)-pyrrolidin-3-yl-urea (Intermediate BD).

### Example 58

### 4-[(R)-3-(3-{(R)-1-[6-(2,2-Diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-ureido)-pyrrolidine-1-carbonyl]-benzoic acid methyl ester trifluoroacetate

A suspension comprising (2S,3S,4R,5R)-5-{6-(2,2-diphenyl-ethylamino)-2-[(R)-3-((R)-3-pyrrolidin-3-ylureido)-pyrrolidin-1-yl]-purin-9-yl}-3,4-dihydroxy-tetrahydro-furan-2-carboxylic acid ethylamide hydrochloride (Example 57) (0.144 g, 0.2 mmol), methyl-4-chlorocarbonyl benzoate (0.059 g, 0.3 mmol) and TEA (83 μL, 0.6 mmol) in THF (2 mL) and NMP (0.6 mL) is stirred at RT for 3 days. The solvent is removed in vacuo and purification by C-18 reverse phase column chromatography eluting with acetonitrile:water (0.1% TFA) (gradient of 0-100% acetonitrile) affords the title compound.

### Example 59

### 4-[(R)-3-(3-{(R)-1-[6-(2,2-Diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-ureido)-pyrrolidine-1-carbonyl]-benzoic acid trifluoroacetate

A solution of 4-[(R)-3-(3-{(R)-1-[6-(2,2-diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-ureido)-pyrrolidine-1-carbonyl]-benzoic acid methyl ester trifluoroacetate (Example 58) (0.05 g, 0.05 mmol) in MeOH (1 mL) is treated with potassium hydroxide (0.029 g, 0.52 mmol) in water (0.29 mL). The resulting mixture is stirred at RT for 2 hours and the solvent is then removed in vacuo. Purification of the crude product by C-18 reverse phase column chromatography eluting with acetonitrile:water (0.1% TFA) (gradient of 0-100% acetonitrile) affords the title compound.

### Examples 60-64

These compounds namely,
(2R,3R,4S,5R)-2-[(R)-2-[1,3′]Bipyrrolidinyl-1′-yl-6-(2,2-diphenyl-ethylamino)-purin-9-yl]-5-(2-ethyl-2H-tetrazol-5-yl)-tetrahydro-furan-3,4-diol trifluoroacetate (Example 60);
(2R,3R,4S,5R)-2-(6-(2,2-Diphenyl-ethylamino)-2-[(R)-3-(5-methyl-pyridin-2-ylamino)-pyrrolidin-1-yl]-purin-9-yl)-5-(2-ethyl-2H-tetrazol-5-yl)-tetrahydro-furan-3,4-diol trifluoroacetate (Example 61);
(2R,3R,4S,5R)-2-[6-(2,2-Diphenyl-ethylamino)-2-((R)-3-imidazol-1-yl-pyrrolidin-1-yl)-purin-9-yl]-5-(2-ethyl-2H-tetrazol-5-yl)-tetrahydro-furan-3,4-diol trifluoroacetate (Example 62);
2-((R)-1-{6-(2,2-Dipheny-ethylamino)-9-[(2R,3R,4S,5R)-5-(2-ethyl-2H-tetrazol-5-yl)-3,4-dihydroxy-tetrahydro-uran-2-yl]-9H-purin-2-yl}-pyrrolidin-3-yl)-2,3-dihydro-1H-isoindole-5-carboxylic acid methyl ester trifluoroacetate (Example 63); and
N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-nicotinamide trifluoroacetate (Example 64),
   are prepared analogously to Example 11 by replacing (2R,3R,4S,5R)-2-[2-chloro-6-(2,2-diphenyl-ethylamino)-purin-9-yl]-5-hydroxymethyl-tetrahydro-furan-3,4-diol with (2R,3R,4S,5R)-2-[2-chloro-6-(2,2-diphenyl-ethylamino)-purin-9-yl]-5-(2-ethyl-2H-tetrazol-5-yl)-tetrahydro-furan-3,4-diol (WO 98/28319) and by replacing 1,3-di(R)-pyrrolidin-3-yl-urea with the appropriate cyclic amine.

### Examples 65-73

These compounds namely,
(2R,3R,4S,5S)-2-[(R)-2-[1,3′]Bipyrrolidinyl-1′-yl-6-((S)-1-hydroxymethyl-2-phenyl-ethylamino)-purin-9-yl]-5-(3-ethyl-isoxazol-5-yl)-tetrahydro-furan-3,4-diol (Example 65);
(2R,3R,4S,5S)-2-[(R)-2-[1,3′]Bipyrrolidinyl-1′-yl-6-(1-ethyl-propylamino)-purin-9-yl]-5-(3-ethyl-isoxazol-5-yl)-tetrahydro-furan-3,4-diol (Example 66);
N—((R)-1-{6-[2,2-bis-(4-Hydroxy-phenyl)-ethylamino]-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-isonicotinamide (Example 67);
(2R,3R,4S,5S)-2-[(R)-2-[1,3′]Bipyrrolidinyl-1′-yl-6-(2,2-diphenyl-ethylamino)-purin-9-yl]-5-(3-ethyl-isoxazol-5-yl)-tetrahydro-furan-3,4-diol (Example 68);
(2R,3R,4S,5R)-2-[(R)-2-[1,3′]Bipyrrolidinyl-1′-yl-6-((S)-1-hydroxymethyl-2-phenyl-ethylamino)-purin-9-yl]-5-(2-ethyl-2H-tetrazol-5-yl)-tetrahydro-furan-3,4-diol (Example 69);
(2R,3R,4S,5R)-2-[(R)-2-[1,3′]Bipyrrolidinyl-1′-yl-6-(1-ethyl-propylamino)-purin-9-yl]-5-(2-ethyl-2H-tetrazol-5-yl)-tetrahydro-furan-3,4-diol (Example 70);
N—((R)-1-{6-[2,2-bis-(4-Hydroxy-phenyl)-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-isonicotinamide (Example 71);
(2S,3S,4R,5R)-5-[(R)-2-[1,3′]Bipyrrolidinyl-1′-yl-6-(2,2-diphenyl-ethylamino)-purin-9-yl]-3,4-dihydroxy-tetrahydro-furan-2-carboxylic acid ethylamide trifluoroacetate (Example 72); and
N—((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-nicotinamide trifluoroacetate (Example 73),
   are prepared analogously to Example 11 by replacing (2R,3R,4S,5R)-2-[2-chloro-6-(2,2-diphenyl-ethylamino)-purin-9-yl]-5-hydroxymethyl-tetrahydro-furan-3,4-diol with the appropriate intermediate (described herein) and by replacing 1,3-di(R)-pyrrolidin-3-yl-urea with the appropriate 3-(R)-aminopyrrolidine derivative. The preparations of the amines which are not commercially available are described in the intermediates section.

### Example 74

### N-{(R)-1-[6-(2,2-Diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-isonicotinamide

### Step 1

### N-{(R)-1-[6-(2,2-Diphenyl-ethylamino)-9-((3aR,4R,6S,6aS)-6-ethylcarbamoyl-2,2-dimethyl-tetrahydro-furo[3,4-d][1,3]dioxol-4-yl)-9H-purin-2-yl]-pyrrolidin-3-yl}-isonicotinamide trifluoroacetate

This compound is prepared analogously to Example 11 by replacing (2R,3R,4S,5R)-2-[2-chloro-6-(2,2-diphenyl-ethylamino)-purin-9-yl]-5-hydroxymethyl-tetrahydro-furan-3,4-diol with (3aS,4S,6R,6aR)-6-[2-chloro-6-(2,2-diphenyl-ethylamino)-purin-9-yl]-2,2-dimethyl-tetrahydro-furo[3,4-d][1,3]dioxole-4-carboxylic acid ethylamide (WO 96/02553) and by replacing 1,3-di(R)-pyrrolidin-3-yl-urea with (R)—N-pyrrolidin-3-yl-isonicotinamide (Intermediate I).

### Step 2

### N-{(R)-1-[6-(2,2-Diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-isonicotinamide

The title compound is prepared analogously to Example 6 by replacing {(S)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid tert-butyl ester trifluoroacetate with N-{(R)-1-[6-(2,2-diphenyl-ethylamino)-9-((3aR,4R,6S,6aS)-6-ethylcarbamoyl-2,2-dimethyl-tetrahydro-furo[3,4-d][1,3]dioxol-4-yl)-9H-purin-2-yl]-pyrrolidin-3-yl}-isonicotinamide trifluoroacetate.

### Example 75

### 1-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-3-yl-urea trifluoroacetate

### Step 1

### (2R,3R,4S,5R)-2-[2-((R)-3-Amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate

This compound is prepared analogously to Example 6 using ((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-carbamic acid tert-butyl ester which is prepared from Intermediate AL and (R)-pyrrolidin-3-yl-carbamic acid tert-butyl ester.

### Step 2

### 1-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-3-yl-urea trifluoroacetate

A solution comprising (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate (20.4 mg, 0.034 mmol) and 3-pyridyl isocyanate (4.1 mg, 0.034 mmol) in chloroform/DMSO (1 mL) is stirred at RT for 3 hours. Purification by C-18 reverse phase column chromatography eluting with acetonitrile:water (0.1% TFA) (gradient of 0-100% acetonitrile) to afford the titled compound.

### Example 76

### N—{(R)-1-[6-(2,2-Diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-6-morpholin-4-yl-nicotinamide trifluoroacetate

### Step 1

### (2S,3S,4R,5R)-5-[2-((R)-3-Amino-pyrrolidin-1-yl)-6-(2,2-diphenyl-ethylamino)-purin-9-yl]-3,4-dihydroxy-tetrahydro-furan-2-carboxylic acid ethylamide

This compound is prepared from Intermediate J analogously to (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate (Example 75, Step 1).

### Step 2

### N-{(R)-1-[6-(2,2-Diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-6-morpholin-4-yl-nicotinamide trifluoroacetate

A reaction mixture comprising (2S,3S,4R,5R)-5-[2-((R)-3-amino-pyrrolidin-1-yl)-6-(2,2-diphenyl-ethylamino)-purin-9-yl]-3,4-dihydroxy-tetrahydro-furan-2-carboxylic acid ethylamide (Step 1) (30 mg, 0.052 mmol) and 6-morpholinonicotinyl chloride (35 mg, 0.156 mmol) in THF (1 mL) is treated with TEA (134 μL, 0.96 mmol) and stirred at room temperature for 5 days. The resulting mixture is diluted with THF (4 mL) and then filtered. The filtrate is concentrated in vacuo and then treated with DMSO (0.4 mL). The resulting suspension is filtered again and purified by preparative HPLC to afford the title compound.

### Examples 77-79

These compounds namely,
N—((R)-1-{6-[2,2-bis-(4-Hydroxy-phenyl)-ethylamino]-g-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-6-morpholin-4-yl-nicotinamide trifluoroacetate (Example 77);
N—((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-6-morpholin-4-yl-nicotinamide trifluoroacetate (Example 78); and
N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-6-morpholin-4-yl-nicotinamide trifluoroacetate (Example 79),
   are prepared analogously to Example 76 by replacing Intermediate J with the appropriate intermediate.

### Example 80

### (2R,3R,4S,5R)-2-{6-(2,2-Diphenyl-ethylamino)-2-[(R)-3-(4-fluoro-phenyl)-pyrrolidin-1-yl]-purin-9-yl}-5-(2-ethyl-2H-tetrazol-5-yl)-tetrahydro-furan-3,4-diol trifluoroacetate

This compound is prepared analogously to Example 1 by replacing (2R,3R,4S,5R)-2-[2-chloro-6-(2,2-diphenyl-ethylamino)-purin-9-yl]-5-hydroxymethyl-tetrahydro-furan-3,4-diol (Intermediate AA) with (2R,3R,4S,5R)-2-[2-chloro-6-(2,2-diphenyl-ethylamino)-purin-9-yl]-5-(2-ethyl-2H-tetrazol-5-yl)-tetrahydro-furan-3,4-diol (Intermediate AL) and by replacing 4-(4-fluoro-phenyl)-piperidine (Intermediate BA) with (R)-3-(4-fluoro-phenyl)-pyrrolidine (Intermediate K).

### Examples 81-83

These compounds namely,
4-[9-((2R,3R,4S,5R)-3,4-Dihydroxy-5-hydroxymethyl-tetrahydro-furan-2-yl)-6-(2,2-diphenyl-ethylamino)-9H-purin-2-yl]-piperazin-2-one trifluoroacetate (Example 81);
(2R,3R,4S,5R)-2-[6-(2,2-Diphenyl-ethylamino)-2-((R)-3-imidazol-1-yl-pyrrolidin-1-yl)-purin-9-yl]-5-hydroxymethyl-tetrahydro-furan-3,4-diol trifluoroacetate (Example 82); and
(2R,3R,4S,5R)-2-[(R)-2-[1,3′]Bipyrrolidinyl-1-yl-6-(2,2-diphenyl-ethylamino)-purin-9-yl]-5-hydroxymethyl-tetrahydro-furan-3,4-diol trifluoroacetate (Example 83),
   are prepared analogously to Example 11 by replacing 1,3-di(R)-pyrrolidin-3-yl-urea with the appropriate amine.

### Example 84

### (2S,3S,4R,5R)-5-{6-(2,2-Diphenyl-ethylamino)-2-[(R)-3-(3-pyridin-4-ylmethyl-ureido)-pyrrolidin-1-yl]-purin-9-yl}-3,4-dihydroxy-tetrahydro-furan-2-carboxylic acid ethylamide trifluoroacetate

A reaction mixture comprising (2S,3S,4R,5R)-5-[2-((R)-3-amino-pyrrolidin-1-yl)-6-(2,2-diphenyl-ethylamino)-purin-9-yl]-3,4-dihydroxy-tetrahydro-furan-2-carboxylic acid ethylamide (Example 76, Step 1) (19.6 mg, 0.03 mmol), pyridin-4-ylmethyl-carbamic acid phenyl ester (6.5 mg, 0.03 mmol) and DIPEA (18.3 mg, 0.14 mmol) in NMP (0.5 mL) is heated to 110° C. Purification by C-18 reverse phase column chromatography eluting with acetonitrile:water (0.1% TFA) (gradient of 0-100% acetonitrile) affords the title compound.

### Examples 85 and 86

These compounds namely,
1-((R)-1-{6-[2,2-bis-(4-Hydroxy-phenyl)-ethylamino]-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-4-ylmethyl-urea trifluoroacetate (Example 85); and
1-((R)-1-{6-[2,2-bis-(4-Hydroxy-phenyl)-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-4-ylmethyl-urea trifluoroacetate (Example 86),
   are prepared analogously to Example 84 by replacing (2S,3S,4R,5R)-5-[2-((R)-3-amino-pyrrolidin-1-yl)-6-(2,2-diphenyl-ethylamino)-purin-9-yl]-3,4-dihydroxy-tetrahydro-furan-2-carboxylic acid ethylamide (Example 76, Step 1) with the appropriate intermediate (prepared analogously to Example 76, Step 1).

### Examples 87-98

These compounds namely,
(2R,3R,4S,5S)-2-[6-((S)-1-Benzyl-2-hydroxy-ethylamino)-2-((R)-3-dimethylamino-pyrrolidin-1-yl)-purin-9-yl]-5-(3-ethyl-isoxazol-5-yl)-tetrahydro-furan-3,4-diol trifluoroacetate (Example 87);
(2R,3R,4S,5S)-2-[2-((R)-3-Dimethylamino-pyrrolidin-1-yl)-6-(1-ethyl-propylamino)-purin-9-yl]-5-(3-ethyl-isoxazol-5-yl)-tetrahydro-furan-3,4-diol trifluoroacetate (Example 88);
(2R,3R,4S,5S)-2-[2-((R)-3-Dimethylamino-pyrrolidin-1-yl)-6-(2,2-diphenyl-ethylamino)-purin-9-yl]-5-(3-ethyl-isoxazol-5-yl)-tetrahydro-furan-3,4-diol trifluoroacetate (Example 89);
(2R,3R,4S,5R)-2-[6-((S)-1-Benzyl-2-hydroxy-ethylamino)-2-((R)-3-dimethylamino-pyrrolidin-1-yl)-purin-9-yl]-5-(2-ethyl-2H-tetrazol-5-yl)-tetrahydro-furan-3,4-diol trifluoroacetate (Example 90);
(2R,3R,4S,5R)-2-[2-((R)-3-Dimethylamino-pyrrolidin-1-yl)-6-(1-ethyl-propylamino)-purin-9-yl]-5-(2-ethyl-2H-tetrazol-5-yl)-tetrahydro-furan-3,4-diol trifluoroacetate (Example 91);
(2R,3R,4S,5R)-2-[2-((R)-3-Dimethylamino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate (Example 92);
(2R,3R,4S,5R)-2-[6-[2,2-bis-(4-Methoxy-phenyl)-ethylamino]-2-((R)-3-dimethylamino-pyrrolidin-1-yl)-purin-9-yl]-5-(2-ethyl-2H-tetrazol-5-yl)-tetrahydro-furan-3,4-diol trifluoroacetate (Example 93);
(2R,3R,4S,5R)-2-{2-((R)-3-Dimethylamino-pyrrolidin-1-yl)-6-[(naphthalen-1-ylmethyl)-amino]-purin-9-yl}-5-(2-ethyl-2H-tetrazol-5-yl)-tetrahydro-furan-3,4-diol trifluoroacetate (Example 94);
(2R,3R,4S,5R)-2-{2-((R)-3-Dimethylamino-pyrrolidin-1-yl)-6-[(9H-fluoren-9-ylmethyl)-amino]-purin-9-yl}-5-(2-ethyl-2H-tetrazol-5-yl)-tetrahydro-furan-3,4-diol trifluoroacetate (Example 95);
4-(2-{2-((R)-3-Dimethylamino-pyrrolidin-1-yl)-9-[(2R,3R,4S,5R)-5-(2-ethyl-2H-tetrazol-5-yl)-3,4-dihydroxy-tetrahydro-furan-2-yl]-9H-purin-6-ylamino}-ethyl)-benzenesulfonamide trifluoroacetate (Example 96);
(2R,3R,4S,5S)-2-{2-((R)-3-Dimethylamino-pyrrolidin-1-yl)-6-[(naphthalen-1-ylmethyl)-amino]-purin-9-yl}-5-(3-ethyl-isoxazol-5-yl)-tetrahydro-furan-3,4-diol trifluoroacetate (Example 97);
(2R,3R,4S,5S)-2-[6-[2,2-bis-(4-Methoxy-phenyl)-ethylamino]-2-((R)-3-dimethylamino-pyrrolidin-1-yl)-purin-9-yl]-5-(3-ethyl-isoxazol-5-yl)-tetrahydro-furan-3,4-diol trifluoroacetate (Example 98),
   are prepared analogously to Example 1 by replacing (2R,3R,4S,5R)-2-[2-chloro-6-(2,2-diphenyl-ethylamino)-purin-9-yl]-5-hydroxymethyl-tetrahydro-furan-3,4-diol (Intermediate AA) with the appropriate intermediate (the preparations of which are described herein) and by replacing 4-(4-fluoro-phenyl)-piperidine (Intermediate BA) with dimethyl-(R)-pyrrolidin-3-yl-amine.

### Examples 99-110

These compounds namely,
1-((R)-1-{6-((S)-1-Benzyl-2-hydroxy-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate (Example 99);
1-{(R)-1-[9-[(2R,3R,4S,5S)-5-(3-Ethyl-isoxazol-5-yl)-3,4-dihydroxy-tetrahydro-furan-2-yl]-6-(1-ethyl-propylamino)-9H-purin-2-yl]-pyrrolidin-3-yl}-3-(R)-pyrrolidin-3-yl-urea trifluoroacetate (Example 100);
1-((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate (Example 101);
1-((R)-1-{6-((S)-1-Benzyl-2-hydroxy-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate (Example 102);
1-((R)-1-{6-(1-Ethyl-propylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate (Example 103);
1-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate (Example 104);
1-((R)-1-{6-[2,2-bis-(4-methoxy-phenyl)-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate (Example 105);
1-((R)-1-{9-[(2R,3R,4S,5R)-5-(2-Ethyl-2H-tetrazol-5-yl)-3,4-dihydroxy-tetrahydro-furan-2-yl]-6-[(naphthalen-1-ylmethyl)-amino]-9H-purin-2-yl}-pyrrolidin-3-yl)-3-(R)-pyrrolidin-3-yl-urea trifluoroacetate (Example 106);
1-((R)-1-{9-[(2R,3R,4S,5R)-5-(2-Ethyl-2H-tetrazol-5-yl)-3,4-dihydroxy-tetrahydro-furan-2-yl]-6-[(9H-fluoren-9-ylmethyl)-amino]-9H-purin-2-yl}-pyrrolidin-3-yl)-3-(R)-pyrrolidin-3-yl-urea trifluoroacetate (Example 107);
4-(2-{9-[(2R,3R,4S,5R)-5-(2-Ethyl-2H-tetrazol-5-yl)-3,4-dihydroxy-tetrahydro-furan-2-yl]-2-[(R)-3-((R)-3-pyrrolidin-3-ylureido)-pyrrolidin-1-yl]-9H-purin-6-ylamino}-ethyl)-benzenesulfonamide trifluoroacetate (Example 108);
1-((R)-1-{9-[(2R,3R,4S,5S)-5-(3-Ethyl-isoxazol-5-yl)-3,4-dihydroxy-tetrahydro-furan-2-yl]-6-[(naphthalen-1-ylmethyl)-amino]-9H-purin-2-yl}-pyrrolidin-3-yl)-3-(R)-pyrrolidin-3-yl-urea trifluoroacetate (Example 109); and
1-((R)-1-{6-[2,2-bis-(4-Methoxy-phenyl)-ethylamino]-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate (Example 110),
   are prepared analogously to Example 1 by replacing (2R,3R,4S,5R)-2-[2-chloro-6-(2,2-diphenyl-ethylamino)-purin-9-yl]-5-hydroxymethyl-tetrahydro-furan-3,4-diol (Intermediate AA) with the appropriate intermediate (the preparations of which are described herein) and by replacing 4-(4-fluoro-phenyl)-piperidine (Intermediate BA) with 1,3-di-(R)-pyrrolidin-3-yl-urea (Intermediate BD).

### Example 111

### 1-((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-4-ylmethyl-urea trifluoroactetate

### Step 1

### (2R,3R,4S,5S)-2-[2-((R)-3-Amino-pyrrolidin-1-yl)-6-(2,2-diphenyl-ethylamino)-purin-9-yl]-5-(3-ethyl-isoxazol-5-yl)-tetrahydro-furan-3,4-diol trifluoroacetate

This compound is prepared analogously to Example 6 using ((R)-1-{6-(2,2-diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-carbamic acid tert-butyl ester trifluoroacetate which is prepared from Intermediate AH and (R)-pyrrolidin-3-yl-carbamic acid tert-butyl ester.

### Step 2

### 1-((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-4-ylmethyl-urea trifluoroactetate

A solution comprising (2R,3R,4S,5S)-2-[2-((R)-3-amino-pyrrolidin-1-yl)-6-(2,2-diphenyl-ethylamino)-purin-9-yl]-5-(3-ethyl-isoxazol-5-yl)-tetrahydro-furan-3,4-diol trifluoroacetate (21 mg, 0.04 mmol) DIPEA (1 mL) and pyridin-4-ylmethyl-carbamic acid phenyl ester (WO 99/18073) (8 mg, 0.04 mmol) in NMP (1 mL) under an inert atmosphere of Argon is heated to 120° C. overnight. Purification by C-18 reverse phase column chromatography eluting with acetonitrile:water (0.1% TFA) (gradient of 0-100% acetonitrile) to affords the title compound.

### Example 112

### 1-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-4-ylmethyl-urea

This compound is prepared analogously to Example 111 by replacing (2R,3R,4S,5S)-2-[2-((R)-3-amino-pyrrolidin-1-yl)-6-(2,2-diphenyl-ethylamino)-purin-9-yl]-5-(3-ethyl-isoxazol-5-yl)-tetrahydro-furan-3,4-diol trifluoroacetate with (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate (Example 75, Step 1).

### Examples 113 and 114

These compounds namely,
N—((R)-1-{6-(2,2-diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-isonicotinamide trifluoroacetate (Example 113); and
N—((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-isonicotinamide trifluoroacetate (Example 114),
   prepared analogously to Example 11 by replacing (2R,3R,4S,5R)-2-[2-chloro-6-(2,2-diphenyl-ethylamino)-purin-9-yl]-5-hydroxymethyl-tetrahydro-furan-3,4-diol with the appropriate intermediate (described herein) and by replacing 1,3-di(R)-pyrrolidin-3-yl-urea with (R)—N-pyrrolidin-3-yl-isonicotinamide (Intermediate I).

### Example 115

### 1-((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-3-yl-urea trifluoroacetate

This compound is prepared analogously to Example 75 by replacing (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate with (2R,3R,4S,5S)-2-[2-((R)-3-amino-pyrrolidin-1-yl)-6-(2,2-diphenyl-ethylamino)-purin-9-yl]-5-(3-ethyl-isoxazol-5-yl)-tetrahydro-furan-3,4-diol trifluoroacetate (Example 111, Step 1).

### Example 116

### 1-((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-2-ylmethyl-urea trifluoroacetate

A reaction mixture comprising (2R,3R,4S,5S)-2-[2-((R)-3-amino-pyrrolidin-1-yl)-6-(2,2-diphenyl-ethylamino)-purin-9-yl]-5-(3-ethyl-isoxazol-5-yl)-tetrahydro-furan-3,4-diol trifluoroacetate (Example 111, Step 1) (20 mg, 0.034 mmol), phenyl chloroformate (10 mg, 0.069 mmol) and potassium carbonate (9 mg, 0.069 mmol) in THF (0.5 mL) is stirred at RT for 1 hour. Then 2-amino methylpyridine (10 mg, 0.108 mmol) is added the reaction mixture is stirred at RT overnight. DMSO (0.5 mL) is added and the mixture is heated to 100° C. for 1 hour. After cooling to RT, the mixture is purified by C-18 reverse phase column chromatography eluting with acetonitrile:water (0.1% TFA) (gradient of 0-100% acetonitrile) to afford the title compound.

### Examples 117 and 118

These compounds namely,
1-((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-2-ylmethyl-urea trifluoroacetate (Example 117); and
(2S,3S,4R,5R)-5-{6-(2,2-diphenyl-ethylamino)-2-[(R)-3-(3-pyridin-3-yl-ureido)-pyrrolidin-1-yl]-purin-9-yl}-3,4-dihydroxy-tetrahydro-furan-2-carboxylic acid ethylamide trifluoroacetate (Example 118),
   are prepared analogously to Example 116 by replacing (2R,3R,4S,5S)-2-[2-((R)-3-amino-pyrrolidin-1-yl)-6-(2,2-diphenyl-ethylamino)-purin-9-yl]-5-(3-ethyl-isoxazol-5-yl)-tetrahydro-furan-3,4-diol trifluoroacetate (Example 111, Step 1) with (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate and (2S,3S,4R,5R)-5-[2-((R)-3-amino-pyrrolidin-1-yl)-6-(2,2-diphenyl-ethylamino)-purin-9-yl]-3,4-dihydroxy-tetrahydro-furan-2-carboxylic acid ethylamide trifluoroacetate, respectively.

### Example 119

### 1-((R)-1-{6-[2,2-bis-(4-Hydroxy-phenyl)-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-3-yl-urea

This compound is prepared analogously to Example 75 by replacing ((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-carbamic acid tert-butyl ester with (2R,3R,4S,5R)-2-{2-((R)-3-amino-pyrrolidin-1-yl)-6-[2,2-bis-(4-hydroxy-phenyl)-ethylamino]-purin-9-yl}-5-(2-ethyl-2H-tetrazol-5-yl)-tetrahydro-furan-3,4-diol which is prepared from Intermediate AK and (R)-pyrrolidin-3-yl-carbamic acid tert-butyl ester.

### Example 120

### 1-((R)-1-{6-Amino-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea hydrochloride

This compound is prepared analogously to Example 1 by replacing (2R,3R,4S,5R)-2-[2-chloro-6-(2,2-diphenyl-ethylamino)-purin-9-yl]-5-hydroxymethyl-tetrahydro-furan-3,4-diol (Intermediate AA) with (2R,3R,4S,5R)-2-[2-chloro-6-(2,2-diphenyl-ethylamino)-purin-9-yl]-5-(2-ethyl-2H-tetrazol-5-yl)-tetrahydro-furan-3,4-diol (Intermediate AB) and by replacing 4-(4-fluoro-phenyl)-piperidine (Intermediate BA) with 1,3-di-(R)-pyrrolidin-3-yl-urea (Intermediate BD).

### Example 121

### 1-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N-cyano-2-phenyl-isourea

A solution of (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-dioi trifluoroacetate (60 mg, 0.1 mmol) and diphenyl cyanocarbodiimidate (24 mg, 0.1 mmol) in DCM (2.0 mL) is treated with TEA (14 μL, 0.1 mmol) and stirred at RT for 5 hours. The solvent is removed in vacuo and purification of the resulting crude product by chromatography on silica eluting with EtOAc/iso-hexane (0-100%) affords the title product.

### Example 122

### N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N′-cyano-N″-pyridin-2-ylmethyl-guanidine

A solution of 1-((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N-cyano-2-phenyl-isourea (30 mg, 0.04 mmol) and 2-(aminomethyl)pyridine (6 μL, 0.32 mmol) in dry acetonitrile (1.5 mL) is treated with TEA (22 μL, 0.16 mmol) and heated using microwave radiation in a Personal Chemistry Emrys™ Optimizer microwave reactor at 100° C. for 2000 s. The solvent is removed in vacuo and the resulting crude product is partitioned between EtOAc and water. The organic portion is separated, dried (Na₂SO₄) and concentrated in vacuo to afford an orange oil. Purification of the oil by mass directed preparative HPLC affords the trifluoroacetate salt which is converted to the free base product by washing with NaHCO₃/EtOAc.

### Example 123

### N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N′-cyano-N″-pyridin-3-yl-guanidine

A mixture comprising 1-((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N-cyano-2-phenyl-isourea (50 mg, 0.07 mmol) and 3-aminopyridine (7 mg, 0.07 mmol) in dry THF (2 mL) and cat. DMAP is heated using microwave radiation in a Personal Chemistry Emrys™ Optimizer microwave reactor at 120° C. for 1 hour. The solvent is removed in vacuo and the resulting crude product is partitioned between EtOAc and water. The organic portion is separated, dried (Na₂SO₄) and concentrated in vacuo to afford a yellow oil. Purification of the oil by chromatography on silica eluting with EtOAc/iso-hexane (30-100% EtOAc) affords the title product as a yellow solid.

### Example 124

### 3-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-ylamino)-4-methoxy-cyclobut-3-ene-1,2-dione

This compound is prepared analogously to Example 123 by replacing 1-((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N-cyano-2-phenyl-isourea with (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate and by replacing 3-aminopyridine with 3,4-dimethoxy-3-cyclobutene-1,2-dione. The reaction is carried out in absolute EtOH.

### Example 125

### N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-4-hydroxy-benzamidine

This compound is prepared analogously to Example 123 by replacing 1-((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N-cyano-2-phenyl-isourea with (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate and by replacing 3-aminopyridine with ethyl-4-hydroxybenzimidate.

### Example 126

### 3-[N′—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N″-cyano-guanidino]-benzenesulfonamide

This compound is prepared analogously to Example 123 by replacing 3-aminopyridine with 3-aminobenzene sulphonamide.

### Example 127

### N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-oxalamic acid methyl ester

A cooled (0° C.) solution of (2R,3R,4S,5R)-2-[2-((R)-3-amino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate (50 mg, 0.084 mmol), TEA (23 μL, 0.16 mmol) and cat. DMAP in dry THF (3 mL) is treated dropwise with methyl oxalyl chloride (9.2 μL, 0.1 mmol). After 30 minutes, the reaction mixture is allowed to warm to RT and thereafter, quenched by addition of water. The mixture is extracted twice with EtOAc and the combined organic portions are dried (Na₂SO₄) and concentrated in vacuo to afford a yellow oil. Purification of the oil by chromatography on silica eluting with EtOAc/iso-hexane (0-100% EtOAc) affords the title product as a yellow solid.

### Example 128

### N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-oxalamic acid

A solution of N—((R)-1-{6-(2,2-diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-oxalamic acid methyl ester (Example 127) (20 mg, 0.029 mmol) in MeOH (1 mL) is treated with 5 M potassium hydroxide solution (0.5 mL). After stirring at RT for 20 minutes, the solvent is removed in vacuo. The crude residue is dissolved in water and extracted with twice with EtAcO. The aqueous is then acidified to pH 1 with concentrated HCl and re-extracted with EtOAc. The organic portions are combine, dried and concentrated in vacuo to afford the title compound as a yellow solid.

1. A compound of formula (I) 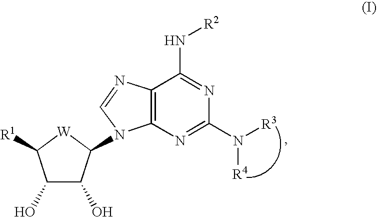
or stereoisomers or pharmaceutically acceptable salts thereof,
wherein
W is selected from CH₂ and O;
R¹ is selected from CH₂OH, CH₂—O—C₁-C₈-alkyl, C(O)—O—C₁-C₈-alkyl, C(O)NH₂, C(O)—NH—C₁-C₈-alkyl and a 3- to 10-membered heterocyclic ring containing at least one ring heteroatom selected from the group consisting of nitrogen, oxygen and sulphur, optionally substituted by C₁-C₈-alkyl;
R² is hydrogen or C₁-C₈-alkyl optionally substituted by hydroxy or C₆-C₁₀-aryl;
R³ and R⁴, together with the nitrogen atom to which they are attached, form a 3- to 10-membered heterocyclic group containing the indicated nitrogen atom as a ring heteroatom and optionally at least one ring heteroatom selected from the group consisting of nitrogen, oxygen and sulphur, optionally substituted by 0-3R⁵;
R⁵ is selected from OH, C₁-C₈-alkyl optionally substituted by OH, C₁-C₈-alkoxy, C₇-C₁₄-aralkyl optionally substituted with OH, O—C₁-C₈-alkyl, halogen C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₁-C₁₀-aryl optionally substituted by OH, C₁-C₈-alkyl, O—C₁-C₈-alkyl or -halogen, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₈-alkylcarbonyl, C₁-C₈-alkoxycarbonyl, di(C₁-C₈-alkyl)aminocarbonyl, COOR^{5k}, C(O)R^{5l} and a 3- to 10-membered heterocyclic group containing at least one ring heteroatom selected from the group consisting of nitrogen, oxygen and sulphur optionally substituted by COOR^{5m};
R^{5a}, R^{5b}, R^{5c}, R^{5f}, R^{5h} and R⁵ⁱ are, independently, H, C₁-C₈-alkyl or C₆-C₁₀-aryl;
R^{5d}, R^{5e}, R^{5g} and R^{5j} are, independently, C₁-C₈-alkyl or a 3- to 10-membered heterocyclic group containing at least one ring heteroatom selected from the group consisting of nitrogen, oxygen and sulphur, optionally substituted by 0-3R⁶;
R^{5k} is H, C₁-C₈-alkyl, C₆-C₁₀-aryl or a 3- to 10-membered heterocyclic group containing at least one ring heteroatom selected from the group consisting of nitrogen, oxygen and sulphur;
R^{5l} is C₁-C₈-alkyl, C₆-C₁₀-aryl, NHR⁷ or a 3- to 10-membered heterocyclic group containing at least one ring heteroatom selected from the group consisting of nitrogen, oxygen and sulphur;
R^{5m} is H, C₁-C₈-alkyl or C₇-C₁₄-aralkyl;
R⁶ is selected from OH, C₁-C₈-alkyl optionally substituted by OH, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, O—C₁-C₈-alkyl or -halogen, NR^{6a}R^{6b}, NHC(O)R^{6c}, NHS(O)₂R^{6d}, NHS(O)₂R^{6e}, NR^{6f}C(O)NR^{6g}R^{6h}, NR⁶ⁱC(O)OR^{6j}, C₁-C₈-alkylcarbonyl, C₁-C₈-alkoxycarbonyl, di(C₁-C₈-alkyl)aminocarbonyl, COOR^{6k}, C(O)R^{6l}, C(O)NHR^{6m} and a 3-10-membered heterocyclic group containing at least one ring heteroatom selected from the group consisting of nitrogen, oxygen and sulphur, optionally substituted by 0-3R⁸;
R^{6a}, R^{6b}, R^{6c}, R^{6f}, R^{6h} and R⁶ⁱ are, independently, H, C₁-C₈-alkyl or C₆-C₁₀-aryl;
R^{6d}, R^{6e}, R^{6g}, R^{6j} and R^{6m} are, independently, C₁-C₈-alkyl or a 3- to 10-membered heterocyclic group containing at least one ring heteroatom selected from the group consisting of nitrogen, oxygen and sulphur, optionally substituted by COOR⁹;
R^{6k} is H, C₁-C₈-alkyl, C₆-C₁₀-aryl or a 3- to 10-membered heterocyclic group containing at least one ring heteroatom selected from the group consisting of nitrogen, oxygen and sulphur;
R^{6f} is C₁-C₈-alkyl, C₆-C₁₀-aryl or a 3- to 10-membered heterocyclic group containing at least one ring heteroatom selected from the group consisting of nitrogen, oxygen and sulphur, optionally substituted by COOR¹⁰;
R⁷ is COOR^{7a} or a 3- to 10-membered heterocyclic group containing at least one ring heteroatom selected from the group consisting of nitrogen, oxygen and sulphur, optionally substituted by COOR^{7b}; and
R^{7a}, R^{7b}, R⁸, R⁹ and R¹⁰ are selected from H, C₁-C₈-alkyl and C₇-C₁₄-aralkyl.

2. A compound of formula (I) according to claim 1, or stereoisomers or pharmaceutically acceptable salts thereof,
wherein
W is selected from CH₂ and O;
R¹ is selected from CH₂OH, C(O)—NH—C₁-C₈-alkyl and a 3- or 10-membered heterocyclic ring containing at least one ring heteroatom selected from the group consisting of nitrogen, oxygen and sulphur optionally substituted by C₁-C₈-alkyl;
R² is hydrogen or C₁-C₈-alkyl optionally substituted by C₆-C₁₀-aryl;
R³ and R⁴, together with the nitrogen atom to which they are attached, form a 3- to 10-membered heterocyclic group containing the indicated nitrogen atom as a ring heteroatom and optionally at least one ring heteroatom selected from the group consisting of nitrogen, oxygen and sulphur, optionally substituted by 0-3R⁵;
R⁵ is selected from OH, C₁-C₈-alkyl optionally substituted by OH, C₁-C₈-alkoxy, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or halogen, O—C₆-C₁₀-aryl optionally substituted by OH, C₁-C₈-alkyl, O—C₁-C₈-alkyl or halogen, NR^{5a}R^{5b}, NHC(O)R^{5c}, NHS(O)₂R^{5d}, NHS(O)₂R^{5e}, NR^{5f}C(O)NR^{5g}R^{5h}, NP⁵ⁱC(O)OR^{5j}, C₁-C₈-alkylcarbonyl, C₁-C₈-alkoxycarbonyl, di(C₁-C₈-alkyl)aminocarbonyl, COOR^{5k}, C(O)R^{5l} and a 3- to 10-membered heterocyclic group containing at least one ring heteroatom selected from the group consisting of nitrogen, oxygen and sulphur optionally substituted by COOR^{5m};
R^{5a}, R^{5b}, R^{5c}, R^{5f}, R^{5h} and R⁵ⁱ are, independently, H, C₁-C₈-alkyl or C₆-C₁₀-aryl;
R^{5d}, R^{5e}, R^{5g} and R^{5j} are, independently, C₁-C₈-alkyl or a 3- to 10-membered heterocyclic group containing at least one ring heteroatom selected from the group consisting of nitrogen, oxygen and sulphur, optionally substituted by 0-3R⁶;
R^{5k} is H, C₁-C₈-alkyl, C₆-C₁₀-aryl or a 3- to 10-membered heterocyclic group containing at least one ring heteroatom selected from the group consisting of nitrogen, oxygen and sulphur;
R^{5l} is C₁-C₈-alkyl, C₆-C₁₀-aryl, NHR⁷ or a 3- to 10-membered heterocyclic group containing at least one ring heteroatom selected from the group consisting of nitrogen, oxygen and sulphur;
R^{5m} is H, C₁-C₈-alkyl or C₇-C₁₄-aralkyl;
R⁶ is selected from OH, C₁-C₈-alkyl optionally substituted by OH, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or halogen, O—C₆-C₁₀-aryl optionally substituted by OH, C₁-C₅-alkyl, O—C₁-C₈-alkyl or halogen, NR^{6a}R^{6b}, NHC(O)R^{6c}, NHS(O)₂R^{6d}, NHS(O)₂R^{6e}, NR^{6f}C(O)NR^{6g}R^{6h}, NR⁶ⁱC(O)OR^{6j}, C₁-C₈-alkylcarbonyl, C₁-C₈-alkoxycarbonyl, di(C₁-C₈-alkyl)aminocarbonyl, COOR^{6k}, C(O)R⁶ⁱ, C(O)NHR^{6m} and a 3- to 10-membered heterocyclic group containing at least one ring heteroatom selected from the group consisting of nitrogen, oxygen and sulphur, optionally substituted by 0-3R⁸;
R^{6a}, R^{6b}, R^{6c}, R^{6f}, R^{6h} and R⁶ⁱ are, independently, H, C₁-C₈-alkyl or C₆-C₁₀-aryl;
R^{6d}, R^{6e}, R^{6g}, R^{6j} and R^{6m} are, independently, C₁-C₈-alkyl or a 3- to 10-membered heterocyclic group containing at least one ring heteroatom selected from the group consisting of nitrogen, oxygen and sulphur, optionally substituted by 0-3R⁹;
R^{6k} is H, C₁-C₈-alkyl, C₆-C₁₀-aryl or a 3- to 10-membered heterocyclic group containing at least one ring heteroatom selected from the group consisting of nitrogen, oxygen and sulphur;
R^{6l} is C₁-C₈-alkyl, C₆-C₁₀-aryl or a 3- to 10-membered heterocyclic group containing at least one ring heteroatom selected from the group consisting of nitrogen, oxygen and sulphur, optionally substituted by COOR¹⁰;
R⁷ is COOR^{7a} or a 3- to 10-membered heterocyclic group containing at least one ring heteroatom selected from the group consisting of nitrogen, oxygen and sulphur, optionally substituted by COOR^{7b}; and
R^{7a}, R^{7b}, R⁸, R⁹ and R¹⁰ are selected from H, C₁-C₈-alkyl and C₇-C₁₄-aralkyl.

3. A compound according to claim 1 or stereoisomers or pharmaceutically acceptable salts thereof, wherein the compound is of formula (II) 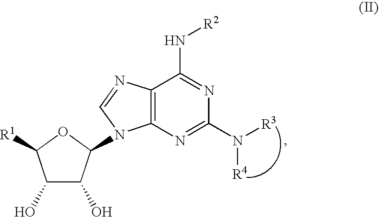
wherein
R¹ is selected from CH₂OH, C(O)—NH—C₁-C₄-alkyl and a 3- to 10-membered heterocyclic ring containing at least one ring heteroatom selected from the group consisting of nitrogen, oxygen and sulphur optionally substituted by C₁-C₈-alkyl;
R² is hydrogen or C₁-C₄-alkyl optionally substituted by C₆-C₈-aryl;
R³ and R⁴, together with the nitrogen atom to which they are attached, form a 3- to 10-membered heterocyclic group containing the indicated nitrogen atom as a ring heteroatom and optionally at least one other ring nitrogen atom, optionally substituted by at least one heteroatom selected from the group consisting of nitrogen, oxygen and sulphur, optionally substituted by 0-3R⁵, the heterocyclic group being saturated or comprising a saturated heterocyclic ring fused to a carbocyclic ring or being a 5-membered unsaturated group;
R⁵ is selected from OH, C₁-C₄-alkyl optionally substituted by OH, C₁-C₄-alkoxy, C₆-C₁₀-aryl optionally substituted by halogen, O—C₆-C₁₀-aryl optionally substituted by halogen, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₄-alkylcarbonyl, C₁-C₄-alkoxycarbonyl, di(C₁-C₄-alkyl)aminocarbonyl, COOR^{5k}, C(O)R^{5l} and a 3- to 10-membered heterocyclic group containing at least one ring heteroatom selected from the group consisting of nitrogen, oxygen and sulphur optionally substituted by COOR^{5m};
R^{5a}, R^{5b}, R^{5c}, R^{5f}, R^{5h} and R⁵ⁱ are, independently, H, C₁-C₄-alkyl or C₆-C₁₀-aryl;
R^{5d}, R^{5e}, R^{5g} and R^{5j} are, independently, C₁-C₄-alkyl or a 3- to 10-membered heterocyclic group containing at least one ring heteroatom selected from the group consisting of nitrogen, oxygen and sulphur, optionally substituted by 0-3R⁶;
R^{5k} is H, C₁-C₄-alkyl or C₆-C₁₀-aryl;
R^{5l} is C₁-C₄-alkyl, C₆-C₁₀-aryl, NHR⁷ or a 3- to 10-membered heterocyclic group containing at least one ring heteroatom selected from the group consisting of nitrogen, oxygen and sulphur;
R^{5m} is H, C₁-C₄-alkyl or C₇-C₁₄-aralkyl;
R⁶ is selected from OH, C₁-C₄-alkyl optionally substituted by OH, C₆-C₁₀-aryl optionally substituted by OH, C₁-C₄-alkyl, O—C₁-C₄-alkyl or halogen, O—C₆-C₁₀-aryl optionally substituted by OH, C₁-C₄-alkyl, O—C₁-C₄-alkyl or halogen, NR^{6a}R^{6b}, NHC(O)R^{6c}, NHS(O)₂R^{6d}, NHS(O)₂R^{6e}, NR^{6f}C(O)NR^{6g}R^{6h}, NR⁶ⁱC(O)OR^{6j}, C₁-C₄-alkylcarbonyl, C₁-C₈-alkoxycarbonyl, di(C₁-C₄-alkyl)aminocarbonyl, COOR^{6k} and C(O)R^{6l}, C(O)NHR^{6m} and a 3- to 10-membered heterocyclic group containing at least one ring heteroatom selected from the group consisting of nitrogen, oxygen and sulphur, optionally substituted by 0-3R⁸;
R^{6a}, R^{6b}, R^{6c}, R^{6f}, R^{6h} and R⁶ⁱ are, independently, H, C₁-C₄-alkyl or C₆-C₁₀-aryl;
R^{6d}, R^{6e}, R^{6g}, R^{6j} and R^{6m} are, independently, C₁-C₄-alkyl or a 3- to 10-membered heterocyclic group containing at least one ring heteroatom selected from the group consisting of nitrogen, oxygen and sulphur, optionally substituted by 0-3R⁹;
R^{6k} is H, C₁-C₄-alkyl, C₆-C₁₀-aryl or a 3- to 10-membered heterocyclic group containing at least one ring heteroatom selected from the group consisting of nitrogen, oxygen and sulphur;
R^{6l} is C₁-C₄-alkyl, C₆-C₁₀-aryl or a 3- to 10-membered heterocyclic group containing at least one ring heteroatom selected from the group consisting of nitrogen, oxygen and sulphur, optionally substituted by COOR¹⁰;
R⁷ is COOR^{7a} or a 3- to 10-membered heterocyclic group containing at least one ring heteroatom selected from the group consisting of nitrogen, oxygen and sulphur, optionally substituted by COOR^{7a}; and
R^{7a}, R^{7b}, R⁸, R⁹ and R¹⁰ are selected from H, C₁-C₄-alkyl and C₇-C₁₄-aralkyl.

4. A compound of formula (I) selected from:
(2R,3R,4S,5R)-2-{6-(2,2-Diphenyl-ethylamino)-2-[4-(4-fluoro-phenyl)-piperidin-1-yl]-purin-9-yl}-5-hydroxymethyl-tetrahydro-furan-3,4-diol;
(2R,3R,4S,5R)-2-{6-(2,2-diphenyl-ethylamino-2-[(R)-3-(4-fluoro-phenyl pyrrolidin-yl]-purin-9-yl}-5-hydroxymethyl-tetrahydro-furan-3,4-diol trifluoroacetate;
{(S)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid tert-butyl ester trifluoroacetate;
(2R,3R,4S,5R)-2-{6-(2,2-diphenyl-ethylamino)-2-[3-(4-methoxy-phenyl)-pyrrolidin-1-yl]-purin-9-yl}-5-hydroxymethyl-tetrahydro-furan-3,4-diol trifluoroacetate;
{(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-carbamic acid tert-butyl ester trifluoroacetate;
(2R,3R,4S,5R)-2-[2-((S)-3-Amino-pyrrolidin-1-yl)-6-(2,2-diphenyl-ethylamino)-purin-9-yl]-5-hydroxymethyl-tetrahydrofuran-3,4-diol trifluoroacetate;
(2R,3R,4S,5R)-2-[2-((R)-3-Amino-pyrrolidin-1-yl)-6-(2,2-diphenyl-ethylamino)-purin-9-yl]-5-hydroxymethyl-tetrahydrofuran-3,4-diol trifluoroacetate;
(2R,3R,4S,5R)-2-[2-((R-3-Amino-piperidin-1-yl)-6-(2,2-diphenyl-ethylamino)-purin-9-yl]-5-hydroxymethyl-tetrahydro-furan-3,4-diol trifluoroacetate;
{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidin-3-yl}-3-(3,4,5,6-tetrahydro-2H-[1,2′]bipyridinyl-4-yl)-urea trifluoroacetate;
4-(3-{(R)-1-[9-((2R,3R,4S,5R)-3,4-Dihydroxy-5-hydromethyl-tetrahydro-furan-2-yl)-6-(2,2-diphenyl-ethylamino)-9H-purin-2-yl]-pyrrolindin-3-yl}-ureido)-3,4,5,6-tetrahydro-2H-[1,2′]bipyridinyl-5′-carboxylic acid ethyl ester trifluoroacetate;
1-{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidin-3-yl}-3-(R)-pyrrolidin-3-yl-urea trifluoroacetate;
(2R,3R,4S,5R)-2-[2-[1,4]diazepan-1-yl-6-(2,2-diphenyl-ethylamino)-purin-9-yl]-5-hydroxymethyl-tetrahydro-furan-3,4-diol trifluoroacetate;
(2R,3R,4S,5R)-2-[6-(2,2-diphenyl-ethylamino)-2-((R)-3-hydroxy-pyrrolidin-1-yl)-purin-9-yl]-5-hydroxymethyl-tetrahydro-furan-3,4-diol;
{(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-piperidin-3-yl}-carbamic acid tert-butyl ester trifluoroacetate;
(2R,3R,4S,5R)-2-[2-(3-dimethylamino-pyrrolidin-1-yl)-6-(2,2-diphenyl-ethylamino)-purin-9-yl]-5-hydroxymethyl-tetrahydro-furan-3,4-diol trifluoroacetate;
{1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-3-yl}-methyl-carbamic acid tert-butyl ester;
5-[9-((2R,3R,4S,5R)-3,4-dihydroxy-5-hydroxymethyl-tetrahydro-furan-2-yl)-6-(2,2-diphenyl-ethylamino)-9H-purin-2-yl]-2,5-diaza-bicyclo[2.2.1]heptane-2-carboxylic acid tert-butyl ester trifluoroacetate;
(2R,3R,4S,5R)-2-[6-(2,2-diphenyl-ethylamino)-2-((S)-2-hydroxymethyl-pyrrolidin-1-yl)-purin-9-yl]-5-hydroxymethyl-tetrahydro-furan-3,4-diol trifluoroacetate;
(2R,3R,4S,1)-2-[6-(2,2-diphenyl-ethylamino)-2-((R)-2-hydroxymethyl-pyrrolidin-1-yl)-purin-9-yl]-5-hydroxymethyl-tetrahydro-furan-3,4-diol;
(2R,3R,4S,5R)-2-[6-(2,2-diphenyl-ethylamino)-2-((R)-3-methylamino-pyrrolidin-1-yl)-purin-9-yl]-5-hydroxymethyl-tetrahydro-furan-3,4-diol trifluoroacetate;
(2R,3R,4S,5R)-2-[2-(3-diethylamino-pyrrolidin-1-yl)-6-(2,2-diphenyl-ethylamino)-purin-9-yl]-5-hydroxymethyl-tetrahydro-furan-3,4-diol trifluoroacetate;
(2R,3R,4S,5R)-2-[2-((R)-3-dimethylamino-pyrrolidin-1-yl)-6-(2,2-diphenyl-ethylamino)-purin-9-yl]-5-hydroxymethyl-tetrahydro-furan-3,4-diol trifluoroacetate;
(R)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-piperidine-3-carboxylic acid ethyl ester trifluoroacetate;
4-{[(R)-3-(3-{(R)-1-[9-((2R,3R,4S,5R)-3,4-Dihydroxy-5-hydroxymethyl-tetrahydrofuran-2-yl)-6-(2,2-diphenyl-ethylamino)-9H-purin-2-yl]pyrrolidine-3-yl}-ureido)-pyrrolidine-1-carbonyl]-amino}-piperidine-1-carboxylic acid benzyl ester trifluoroacetate;
4-(3-{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidin-3-yl}-ureido-3,4,5,6-tetrahydro-2H-[1,2′]bipyridinyl-5′-carboxylic acid trifluoroacetate;
(2R,3R,4S,5R)-2-[6-Amino-2-((R)-3-dimethylamino-pyrrolidin-1-yl)-purin-9-yl]-5-(2-ethyl-2H-tetrazol-5-yl)-tetrahydrofuran-3,4-diol trifluoroacetate;
(2R,3R,4S,5R)-5-{6-Amino-2-[3-(3,4-dichloro-phenoxy)-azetidin-1-yl]-purin-9-yl}-3,4-dihydroxy-tetrahydro-furan-2-carboxylic acid ethylamide trifluoroacetate;
(2R,3R,4S,5R)-5-{6-Amino-2-[(R)-3-(4-fluoro-phenyl)-pyrrolidin-1-yl]-purin-9-yl}-3,4-dihydroxy-tetrahydro-furan-2-carboxylic acid ethylamide trifluoroacetate;
(2R,3R,4S,5R)-2-{2-[3-(4-Chlorobenzyl)-azetidin-1-yl]-6-phenethylamino-purin-9-yl}-5-hydroxymethyl-tetrahydro-furan-3,4-diol;
4-{1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidin-3-yl}-piperazine-1-carboxylic acid benzyl ester trifluoroacetate;
(2R,3R,4S,5R)-2-[6-(2,2-Diphenyl-ethylamino)-2-(3-piperazin-1-yl-pyrrolidin-1-yl)-purin-9-yl]-5-hydroxymethyl-tetrahydro-furan-3,4-diol;
(3R,4R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidine-3,4-diol trifluoroacetate;
(3S,4S)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrrolidine-5,4-diol trifluoroacetate;
(2R,3R,4S,5R)-2-[2-(2,5-Dimethyl-pyrrolidin-1-yl)-6-(2,2-diphenyl-ethylamino)-purin-9-yl]-5-hydroxymethyl-tetrahydro-furan-3,4-diol trifluoroacetate;
(2R,3R,4S,5R)-2-[6-(2,2-Diphenyl-ethylamino)-2-pyrrolidin-1-yl-purin-9-yl]-5-hydroxymethyl-tetrahydro-furan-3,4-diol trifluoroacetate;
{(R)-1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-piperidin-3-yl}-carbamic acid tert-butyl ester trifluoroacetate;
(2R,3R,4S,5R)-2-[2-(2,3-Dihydro-indol-1-yl)-6-(2,2-diphenyl-ethylamino)-purin-9-yl]-5-hydroxymethyl-tetrahydro-furan-3,4-diol trifluoroacetate;
(2R,3R,4S,5R)-2-[2-(1,3-Dihydroisoindol-2-yl)-6-(2,2-diphenyl-ethylamino)-purin-9-yl]-5-hydroxymethyl-tetrahydro-furan-3,4-diol trifluoroacetate;
(2R,3R,4S,5R)-2-[6-(2,2-Diphenyl-ethylamino)-2-imidazol-1-yl-purin-9-yl]-5-hydroxymethyl-tetrahydro-furan-3,4-diol trifluoroacetate;
1-[9-((2R,3R,4S,5R)-3,4-Dihydroxy-5-hydroxymethyl-tetrahydrofuran-2-yl)-6-(2,2-diphenyl-ethylamino)-9H-purin-2-yl]-pyrrolidine-3-carboxylic acid methyl ester trifluoroacetate;
(2R,3R,4S,5R)-2-[2-((3R,4R)-3-Benzyl-4-hydroxymethyl-pyrrolidin-1-yl)-6-(2,2-diphenyl-ethylamino)-purin-9-yl]-5-hydroxymethyl-tetrahydrofuran-3,4-diol trifluoroacetate;
(4-Benzyl-piperidin-1-yl)-{(S)-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-2-yl}-methanone trifluoroacetate;
(2S,4R)-1-[9-((2R,4S,5R)-3,4-Dihydroxy-5-hydroxymethyl-tetrahydro-furan-2-yl)-6-(2,2-diphenyl-ethylamino)-9H-purin-2-yl]-4-hydroxy-pyrrolidine-2-carboxylic acid methyl ester trifluoroacetate;
1-[9-((2R,3R,4S,5R)-3,4-Dihydroxy-5-hydroxymethyl-tetrahydro-furan-2-yl)-6-(2,2-diphenyl-ethylamino)-9H-purin-2-yl]-pyrazolidin-3-one trifluoroacetate;
(2R,3R,4S,5R)-2-[6-(2,2-Diphenyl-ethylamino)-2-((S)-2-pyrrolidin-1-ylmethyl-pyrrolidin-1-yl)-purin-9-yl]-5-hydroxymethyl-tetrahydro-furan-3,4-diol trifluoroacetate;
(2R,3R,4S,5R)-2-[6-(2,2-Diphenyl-ethylamino)-2-((R)-2-phenylaminomethyl-pyrrolidin-1-yl)-purin-9-yl]-5-hydroxymethyl-tetrahydro-furan-3,4-diol trifluoroacetate;
(2R,3R,4S,5R)-2-[6-(2,2-Diphenyl-ethylamino)-2-((R)-2-methoxymethyl-pyrrolidin-1-yl)-purin-9-yl]-5-hydroxymethyl-tetrahydro-furan-3,4-diol trifluoroacetate;
(2R,3R,4S,5R)-2-{6-(2,2-Diphenyl-ethylamino)-2-[(R)-2-(hydroxy-diphenyl-methyl)-pyrrolidin-1-yl]-purin-9-yl}-5-hydroxymethyl-tetrahydro-furan-3,4-diol trifluoroacetate;
(2R,3R,4S,5R)-2-{6-(2,2-Diphenyl-ethylamino)-2-[(1S,4S)-5-(4-fluoro-phenyl)-2,5-diaza-bicyclo[2.2.1]hept-2-yl]-purin-9-yl}-5-hydroxymethyl-tetrahydro-furan-3,4-diol trifluoroacetate;
(2R,3R,4S,5R)-2-[6-(2,2-Diphenyl-ethylamino)-2-piperazin-1-yl-purin-9-yl]-5-hydroxymethyl-tetrahydro-furan-3,4-diol trifluoroacetate;
{4-[9-((2R,3R,4S,5R)-3,4-Dihydroxy-5-hydroxymethyl-tetrahydro-furan-2-yl)-6-(2,2-diphenyl-ethylamino)-9H-purin-2-yl]-piperazin-1-yl}-furan-2-yl-methanone trifluoroacetate;
(2R,3R,4S,5R)-2-[6-(2,2-Diphenyl-ethylamino)-2-(4-methyl-[1,4]diazepan-1-yl)-purin-9-yl]-5-hydroxymethyl-tetrahydro-furan-3,4-diol trifluoroacetate;
(2R,3R,4S,5R)-2-[6-(2,2-Diphenyl-ethylamino)-2-(3-pyridin-4-yl-pyrrolidin-1-yl)-purin-9-yl]-5-hydroxymethyl-tetrahydro-furan-3,4-diol trifluoroacetate;
{(2S,4R)-4-tert-Butoxy-1-[9-((2R,3R,4S,5R)-3,4-dihydroxy-5-hydroxymethyl-tetrahydro-furan-2-yl)-6-(2,2-diphenyl-ethylamino)-9H-purin-2-yl]-pyrrolidin-2-ylmethyl}-carbamic acid tert-butyl ester trifluoroacetate;
(2R,3R,4S,5R)-2-[2-[(R)-2-(4-Benzyl-piperidin-1-ylmethyl)-pyrrolidin-1-yl]-6-(2,2-diphenyl-ethylamino)-purin-9-yl]-5-hydroxymethyl-tetrahydro-furan-3,4-diol trifluoroacetate;
(2R,3R,4S,5R)-2-[2-((3S,4S)-3-Benzyl-4-hydroxymethyl-pyrrolidin-1-yl)-6-(2,2-diphenyl-ethylamino)-purin-9-yl]-5-hydroxymethyl-tetrahydro-furan-3,4-diol trifluoroacetate;
(2S,3S,4R,5R)-5-{6-(2,2-Diphenyl-ethylamino)-2-[(R)-3-((R)-3-pyrrolidin-3-ylureido)-pyrrolidin-1-yl]-purin-9-yl}-3,4-dihydroxy-tetrahydro-furan-2-carboxylic acid ethylamide hydrochloride;
4-[(R)-3-(3-{(R)-1-[6-(2,2-Diphenylethylamino)-9-((2R,3R,4S,5S)-5-ethylcarbamyl-3,4-dihydroxy-tetrahydrofuran-2-yl)-9H-purin-2-yl]-pyrrolidin-3-yl}-ureido)-pyrrolidine-1-carbonyl]benzoic acid methyl ester trifluoroacetate;
4-[(R)-3-(3-{(R)-1-[6-(2,2-Diphenylethylamino)-9-((2R,3R,4S,5S)-5-ethylcarbamoyl-3,4-dihydroxy-tetrahydro-furan-2-yl)-9H-purin-2-yl]-pyrrolidin-3-yl}-ureido)-pyrrolidine-1-carbonyl]-benzoic acid trifluoroacetate;
(2R,3R,4S,5R)-2-[(R)-2-[1,3′]Bipyrrolidinyl-1′-yl-6-(2,2-diphenyl-ethylamino)-purin-9-yl]-5-(2-ethyl-2H-tetrazol-5-yl)-tetrahydro-furan-3,4-diol trifluoroacetate;
(2R,3R,4S,5R)-2-{6-(2,2-Diphenyl-ethylamino)-2-[(R)-3-(5-methyl-pyridin-2-ylamino)-pyrrolidin-1-yl]-purin-9-yl}-5 (2-ethyl-2H-tetrazol-5-yl)-tetrahydro-furan-3,4-diol trifluoroacetate;
(2R,3R,4S,5R)-2-[6-(2,2-Diphenyl-ethylamino)-2-((R)-3-imidazol-1-yl-pyrrolidin-1-yl)-purin-9-yl]-5-(2-ethyl-2H-tetrazol-5-yl)-tetrahydro-furan-3,4-diol trifluoroacetate;
2-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-2,3-dihydro-1H-isoindole-5-carboxylic acid methyl ester trifluoroacetate;
N—((R)-1-{6-(2,2-Diphenyl-ethylamino)-9-[(2R,3R,4S,5R)-5 (2-ethyl-2H-tetrazol-5-yl)-3,4-dihydroxy-tetrahydro-furan-2-yl]-9H-purin-2-yl}-pyrrolidin-3-yl)-nicotinamide trifluoroacetate;
(2R,3R,4S,5S)-2-[(R)-2-[1,3′]Bipyrrolidinyl-1′-yl-6-((S)-1-hydroxymethyl-2-phenyl-ethylamino)-purin-9-yl]-5-(3-ethyl-isoxazol-5-yl)-tetrahydro-furan-3,4-diol;
(2R,3R,4S,5S)-2-[(R)-2-[1,3′]Bipyrrolidinyl-1′-yl-6-(1-ethyl-propylamino)-purin-9-yl]-5-(3-ethyl-isoxazol-5-yl)-tetrahydro-furan-3,4-diol;
N—((R)-1-{6-[2,2-bis-(4-Hydroxy-phenyl)-ethylamino]-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-isonicotinamide;
(2R,3R,4S,5S)-2-[(R)-2-[1,3′]Bipyrrolidinyl-1′-yl-6-(2,2-diphenyl-ethylamino)-purin-9-yl]-5-(3-ethyl-isoxazol-5-yl)-tetrahydro-furan-3,4-diol;
(2R,3R,4S,5R)-2-[(R)-2-[1,3′]Bipyrrolidinyl-1′-yl-G-((S)-1-hydroxymethyl-2-phenyl-ethylamino)-purin-9-yl]-5-(2-ethyl-2H-tetrazol-5-yl)-tetrahydro-furan-3,4-diol;
(2R,3R,4S,5R)-2-[(R)-2-[1,3′]Bipyrrolidinyl-1′-yl-6-(1-ethyl-propylamino)-purin-9-yl]-5-(2-ethyl-2H-tetrazol-5-yl)-tetrahydro-furan-3,4-diol;
N—((R)-1-{6-[2,2-bis-(4-Hydroxy-phenyl)-ethylamino]-9-[(2R,3R,4,5R)-5-(2-ethyl-2H-tetrazol-5-yl)-3,4-dihydroxy-tetrahydro-furan-2-yl]-9H-purin-2-yl}-pyrrolidin-3-yl)-isonicotinamide;
(2S,3S,4R,5R)-5-[(R)-2-[1,3′]Bipyrrolidinyl-1-yl-6-(2,2-diphenyl-ethylamino)-purin-9-yl]-3,4-dihydroxy-tetrahydro-furan-2-carboxylic acid ethylamide trifluoroacetate;
N—((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-nicotinamide trifluoroacetate;
N-{(R)-1-[6-(2,2-Diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-isonicotinamide;
1-(R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-3-yl-urea trifluoroacetate;
N-{(R)-1-[6-(2,2-Diphenyl-ethylamino)-9-((2R,3R,4S,5S)-5-ethylcarbamoyl-3,4-dihydroxy-tetrahydro-furan-2-yl)-9H-purin-2-yl]-pyrrolidin-3-yl}-6-morpholin-4-yl-nicotinamide trifluoroacetate;
N—((R)-1-{6-[2,2-bis-(4-Hydroxy-phenyl)-ethylamino]-9-[(2R,3R,4S,5R)-(2-ethyl-2H-tetrazol-5-yl)-3,4-dihydroxy-tetrahydro-furan-2-yl]-9H-purin-2-yl}-pyrrolidin-3-yl)-6-morpholin-4-yl-nicotinamide trifluoroacetate;
N—((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-6-morpholin-4-yl-nicotinamide trifluoroacetate;
N—((R)-1-{6-(2,2-Diphenyl-ethylamino)-9-[(2R,3R,4S,5R)-(2-ethyl-2H-tetrazol-5-yl)-3,4-dihydroxy-tetrahydro-furan-2-yl]-9H-purin-2-yl}-pyrrolidin-3-yl)-6-morpholin-4-yl-nicotinamide trifluoroacetate;
(2R,3R,4S,5R)-2-{6-(2,2-Diphenyl-ethylamino)-2-[(R)-3-(4-fluoro-phenyl)-pyrrolidin-1-yl]purin-9-yl}-5-(2-ethyl-2H-tetrazol-5-yl)-tetrahydro-furan-3,4-diol trifluoroacetate;
4-[9-((2R,3R,4S,5R)-3,4-Dihydroxy-5-hydroxymethyl-tetrahydro-furan-2-yl)-6-(2,2-diphenyl-ethylamino)-9H-purin-2-yl]-piperazin-2-one trifluoroacetate;
(2R,3R,4S,5R)-2-[6-(2,2-Diphenyl-ethylamino)-2-((R)-3-imidazo-1-yl-pyrrolidin-1-yl)-purin-9-yl]-5-hydroxymethyl-tetrahydro-furan-3,4-diol trifluoroacetate;
(2R,3R,4S,5R)-2-[(R)-2-[1,3′]Bipyrrolidinyl-1-yl-6-(2,2-diphenyl-ethylamino)-purin-9-yl]-5-hydroxymethyl-tetrahydro-furan-3,4-diol trifluoroacetate;
(2S,3S,4R,5R)-5-{6-(2,2-Diphenyl-ethylamino)-2-[(R)-3-(3-pyridin-4-ylmethyl-ureido)-pyrrolidin-1-yl]-purin-9-yl}-3,4-dihydroxy-tetrahydro-furan-2-carboxylic acid ethyl amide trifluoroacetate;
1-((R)-1-{6-[2,2-bis-(4-Hydroxy-phenyl)-ethylamino]-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-4-ylmethyl-urea trifluoroacetate;
1-((R)-1-{6-[2,2-bis-(4-Hydroxy-phenyl)-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-4-ylmethyl-urea trifluoroacetate;
(2R,3R,4S,5S)-2-[6-((S)-1-Benzyl-2-hydroxy-ethylamino)-2-((R)-3-dimethylamino-pyrrolidin-1-yl)-purin-9-yl]-5-(3-ethyl-isoxazol-5-yl)-tetrahydrofuran-3,4-diol trifluoroacetate;
(2R,3R,4S,5S)-2-[2-((R)-3-Dimethylamino-pyrrolidin-1-yl)-6-(1-ethyl-propylamino)-purin-9-yl]-5-(3-ethyl-isoxazol-5-yl)-tetrahydro-furan-3,4-diol trifluoroacetate;
(2R,3R,4S,5S)-2-[2-((R)-3-Dimethylamino-pyrrolidin-1-yl)-6-(2,2-diphenylethylamino)-purin-9-yl]-5-(3-ethyl-isoxazol-5-yl)-tetrahydro-furan-3,4-diol trifluoroacetate;
(2R,3R,4S,5R)-2-[6-((S)-1-Benzyl-2-hydroxy-ethylamino)-2-((R)-3-dimethylamino-pyrrolidin-1-yl)-purin-9-yl]-5-(2-ethyl-2H-tetrazol-5-yl)-tetrahydro-furan-3,4-diol trifluoroacetate;
(2R,3R,4S,5R)-2-[2-((R)-3-Dimethylamino-pyrrolidin-1-yl)-6-(1-ethyl-propylamino)-purin-9-yl]-5-(2-ethyl-2H-tetrazol-5-yl)-tetrahydro-furan-3,4-diol trifluoroacetate;
(2R,3R,4S,5R)-2-[2-((R)-3-Dimethylamino-pyrrolidin-1-yl)-6-(2,2-diphenyl-ethylamino)-purin-9-yl]-5-(2-ethyl-2H-tetrazol-5-yl)-tetrahydro-furan-3,4-diol trifluoroacetate;
(2R,3R,4S,5R)-2-[6-[2,2-bis-(4-Methoxy-phenyl)-ethylamino]-2-((R)-3-dimethylamino-pyrrolidin-1-yl)-purin-9-yl-5-(2-ethyl-2H-tetrazol-5-yl)-tetrahydro-furan-3,4-diol trifluoroacetate;
(2R,3R,4S,5R)-2-{2-((R)-3-Dimethylamino-pyrrolidin-1-yl)-6-[(naphthalen-ylmethyl)-amino]-purin-9-yl}-5-(2-ethyl-2H-tetrazol-5-yl)-tetrahydro-furan-3,4-diol trifluoroacetate;
(2R,3R,4S,5R)-2-{2-((R)-3-Dimethylamino-pyrrolidin-1-yl)-6-[(9H-fluoren-9-ylmethyl)-amino]-purin-9-yl}-5-(2-ethyl-2H-tetrazol-5-yl)-tetrahydro-furan-3,4-diol trifluoroacetate;
4-(2-{2-((R)-3-Dimethylamino-pyrrolidin-1-yl)-9-[(2R,3R,4S,5R)-5-(2-ethyl-2H-tetrazol-5-yl)-3,4-dihydroxy-tetrahydro-furan-2-yl]-9H-purin-6-ylamino}-ethyl)-benzenesulfonamide trifluoroacetate;
(2R,3R,4S,5S)-2-{2-((R)-3-Dimethylamino-pyrrolidin-1-yl)-6-[(naphthalen 1-ylmethyl)-amino]-purin-9-yl}-5-(3-ethyl-isoxazol-5-yl)-tetrahydro-furan-3,4-diol trifluoroacetate;
(2R,3R,4S,5S)-2-[6-[2,2-bis-(4-Methoxy-phenyl)-ethylamino]-2-((R)-3-dimethylamino-pyrrolidin-1-yl)-purin-9-yl]-5-(3-ethyl-isoxazol-5-yl)-tetrahydro-furan-3,4-diol trifluoroacetate;
1-((R)-1-{6-((S)-1-Benzyl-2-hydroxy-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate;
1-{(R)-1-[9-(2R,3R,4S,5S)-5-(3-Ethyl-isoxazol-5-yl)-3,4-dihydroxy-tetrahydro-furan-2-yl]-6-(1-ethyl-propylamino)-9H-purin-2-yl]-pyrrolidin-3-yl}-3-(R)-pyrrolidin-3-yl-urea trifluoroacetate;
1-((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate;
1-((R)-1-{6-((S)-1-Benzyl-2-hydroxy-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate;
1-((R)-1-{6-(1-Ethyl-propylamino)-9-[(2R,3S,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate;
1-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate;
1-((R)-1-{6-[2,2-bis-(4-methoxy-phenyl)-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-(R)-pyrrolidin-3-yl-urea trifluoroacetate;
1-((R)-1-{9-[(2R,3R,4S,5R)-5-(2-Ethyl-2H-tetrazol-5-yl)-3,4-dihydroxy-tetrahydro-furan-2-yl]-6-[(naphthalen-1-ylmethyl)amino]-9H-purin-2-yl}-pyrrolidin-3-yl)-3-(R)-pyrrolidin-3-yl-urea trifluoroacetate;
1-((R)-1-{9-[(2R,3R,4S,5R)-5-(2-Ethyl-2H-tetrazol-5-yl)-3,4-dihydroxy-tetrahydro-furan-2-yl]-6-[(9H-fluoren-9-ylmethyl)-amino]-9H-purin-2-yl}pyrrolidin-3-yl)-3-(R)-pyrrolidin-3-yl-urea trifluoroacetate;
4-(2-{9-[(2R,3R,4S,5R)-5-(2-Ethyl-2H-tetrazol-5-yl)-3,4-dihydroxy-tetrahydro-furan-2-yl]-2-[(R)-3-((R)-3-pyrrolidin-3-yl-ureido)-pyrrolidin-1-yl]-9H-purin-6-ylamino}-ethyl)-benzenesulfonamide trifluoroacetate;
1-((R)-1-{9-[(2R,3R,4S,5S)-5-(3-Ethyl-isoxazol-5-yl)-3,4-dihydroxy-tetrahydro-furan-2-yl]-6-[(naphthalen-1-ylmethyl)-amino]-9H-purin-2-yl}-pyrrolidin-3-yl)-3-(R)-pyrrolidin-3-yl-urea trifluoroacetate;
1-((R)-1-{6-[2,2-bis-(4-Methoxy-phenyl)-ethylamino]-9-[(2R,3R,4S,5S)-5-(3-ethyl-isoxazol-5-yl)-3,4-dihydroxy-tetrahydro-furan-2-yl]-9H-purin-2-yl}pyrrolidin-3-yl)-3-(R)-pyrrolidin-3-yl-urea trifluoroacetate;
1-((R)-1-{6-(2,2-Diphenyl-ethylamino)-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-4-ylmethyl-urea trifluoroacetate;
1-(R)-1-{6-(2,2-Diphenyl-ethylamino)-9-[(2R,3R,4,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-4-ylmethyl-urea;
N—((R)-1-{6-(2,2-diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-isonicotinamide trifluoroacetate;
N—((R)-1-{6-(2,2-diphenyl-ethylamino)-9-[(2R,3R,4S,5R)-5-(2-ethyl-2f-tetrazol-5-yl)-3,4-dihydroxy-tetrahydro-furan-2-yl]-9H-purin-2-yl}-pyrrolidin-3-yl)-isonicotinamide trifluoroacetate;
1-((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-3-yl-urea trifluoroacetate;
1-((R)-1-{6-(2,2-Diphenyl-ethylamino)-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-2-ylmethyl-urea trifluoroacetate;
1-((R)-1-{6-(2,2-diphenyl-ethylamino)-9-[(2R,3R,4S,5R)-5-(2-ethyl-2H-tetrazol-5-yl-3,4-dihydroxy-tetrahydro-furan-2-yl]-9H-purin-2-yl}-pyrrolidin-3-yl)-3-pyridin-2-ylmethyl-urea trifluoroacetate;
(2S,3S,4R,5R)-5-{6-(2,2-diphenyl-ethylamino)-2-[(R)-3-(3-pyridin-3-yl-ureido)-pyrrolidin-1-yl]-purin-9-yl}-3,4-dihydroxy-tetrahydro-furan-2-carboxylic acid ethylamide trifluoroacetate;
1-((R)-1-{6-[2,2-bis-(4-Hydroxy-phenyl)-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-3-yl-urea;
1-((R)-1-{6-Amino-9-[(2R,3R,4S,5R)-5-(2-ethyl-2H-tetrazol-5-yl)-3,4-dihydroxy-tetrahydrofuran-2-yl]-9H-purin-2-yl}-pyrrolidin-3-yl)-3-(P)-pyrrolidin-3-yl-urea hydrochloride;
1-((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N-cyano-2-phenyl-isourea;
N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N′-cyano-N″-pyridin-2-ylmethyl-guanidine;
N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N′-Cyano-N″-pyridin-3-yl-guanidine;
3-((R)-1-(6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl)pyrrolidin-3-ylamino)-4-methoxy cyclobut-3-ene-1,2-dione;
N—((R)—-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-4-hydroxy-benzamidine;
3-[N′—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-N″-cyano-guanidino]-benzenesulfonamide;
N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-oxalamic acid methyl ester; and
N—((R)-1-{6-(2,2-Diphenyl-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-oxalamic acid.

5. (canceled)

6. A compound according to claim 1 in combination with an anti-inflammatory, bronchodilatory, anti-histamine or anti-tussive drug substance, said compound and said drug substance being in the same or different pharmaceutical composition.

7. A pharmaceutical composition comprising as active ingredient a compound according to claim 1, optionally together with a pharmaceutically acceptable diluent or carrier.

8. Use of a compound according to claim 1 for the manufacture of a medicament for the treatment of a condition mediated by activation of the adenosine A2A receptor.

9. Use of a compound according to claim 1 for the manufacture of a medicament for the treatment of an inflammatory or obstructive airways disease.

10. A process for the preparation of compounds of formula (I) as defined in claim 1, or stereoisomers or pharmaceutically acceptable salts thereof, which comprises the steps of:
(i) reacting a compound of formula (III) 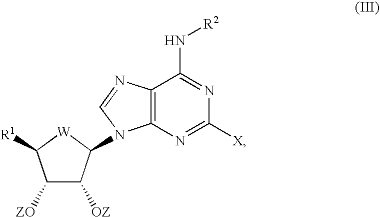
wherein
R¹, R² and W are as defined in claim 1;
Z is H or a protecting group; and
X is a leaving group,
with a compound of formula (IV) 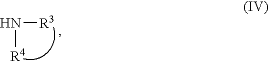
wherein R³ and R⁴ are as defined in claim 1; and
(ii) removing any protecting groups and recovering the resultant compound of formula (I) in free or pharmaceutically acceptable salt form.
